(19) United States
(12) Patent Application Publication
(10) Pub. No.: US 2024/0076666 A1
(43) Pub. Date: Mar. 7, 2024

(54) Immunomodulators Targeting MORC3 for Interferon Induction

(71) Applicant: The Regents of the University of California, Oakland, CA (US)

(72) Inventors: Russell Eaton Vance, Albany, CA (US); Moritz Matthias Gaidt, Berkeley, CA (US)

(21) Appl. No.: 18/263,760

(22) PCT Filed: Feb. 11, 2022
(86) PCT No.: PCT/US2022/016162

§ 371 Date: Aug. 1, 2023

Related U.S. Application Data
(60) Provisional application No. 63/148,775, filed on Feb. 12, 2021.

(51) Int. Cl. C12N 15/113; A61K 38/16; A61K 45/06; A61P 37/02

(52) U.S. Cl. CPC C12N 15/113; A61K 38/162; A61K 45/06; A61P 37/02

(57) ABSTRACT

Disclosed herein are methods of increasing or decreasing endogenous interferon amounts in subjects which comprise inhibiting, reducing, increasing, enhancing, or stabilizing MORC3 in the subjects.

### CROSS-REFERENCE TO RELATED APPLICATIONS

This application claims the benefit of U.S. Patent Application No. 63/148,775, filed Feb. 12, 2021, which is herein incorporated by reference in its entirety.

### ACKNOWLEDGEMENT OF GOVERNMENT SUPPORT

This invention was made with government support under Grant Number A1075039 awarded by the National Institutes of Health. The Government has certain rights in the invention.

### REFERENCE TO A SEQUENCE LISTING SUBMITTED VIA EFS-WEB

The content of the ASCII text file of the sequence listing named “20220211 034044 224W01 ST25” which is 30.4 kb in size was created on Feb. 11, 2022, and electronically submitted via EFS-Web herewith the application is incorporated herein by reference in its entirety.

### BACKGROUND OF THE INVENTION

Type I interferons (IFNs) are cytokines that have potent immunomodulatory effects. For example, IFNs induce anti-viral responses, suppress autoimmune inflammation, and activate cytotoxic lymphocytes. The innate immune system detects pathogens by employing germ-line-encoded pattern-recognition-receptors (PRRs) that sense pathogen-associated molecular patterns (PAMPs) such as lipopolysaccharide and nucleic acids.

Many viruses such as Herpes Simplex Virus-1 (HSV-1) are recognized as a PAMP by the cyclic-GMP-AMP synthase (cGAS)—stimulator of interferon genes (STING) PRR-pathway. Downstream of DNA recognition, STING activates TANK Binding Kinase 1 (TBK1) and I-kappa-B kinase c (IKKc) that phosphorylate and activate interferon regulatory factors 3 and 7 (IRF3/7), leading to transcriptional induction and secretion of type I interferon (IFN) cytokines. IFNs bind to the IFN alpha and beta receptor (IFNAR) to induce transcription of anti-viral interferon-stimulated genes (ISGs).

### SUMMARY OF THE INVENTION

In some embodiments, the present invention is directed to a method of increasing or decreasing the amount of endogenous interferon in a subject, which comprises administering to the subject a MORC3 therapeutic agent or modulating the activity of MRE in the subject. In some embodiments, the MORC3 therapeutic agent is an siRNA, an ATPase inhibitor (e.g., a small molecule ATPase inhibitor), a MORC3 protein, or an ICP0 protein. In some embodiments, the MORC3 therapeutic agent is an siRNA, an ATPase inhibitor (e.g., a small molecule ATPase inhibitor), a MORC3 protein having at least about 90% sequence identity to SEQ ID NO: 1, or an ICP0 protein having at least about 95% sequence identity to SEQ ID NO: 2. In some embodiments, the MORC3 therapeutic agent is an siRNA, an ATPase inhibitor (e.g., a small molecule ATPase inhibitor), a MORC3 protein, or an ICP0 protein. In some embodiments, the MORC3 therapeutic agent is a MORC3 inhibitor. In some embodiments, the MORC3 therapeutic agent is a MORC3 activator. In some embodiments, the MORC3 therapeutic agent stabilizes expression of MORC3. In some embodiments, the MORC3 protein comprises a sequence that has at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 1. In some embodiments, the ICP0 protein comprises a sequence that has at least about 95%, at least about 96%, at least about 97%, at least about 98%, at least about 99%, or 100% sequence identity to SEQ ID NO: 2. In some embodiments, the sequence of the MRE in the subject at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 3. In some embodiments, the subject is in need thereof.

In some embodiments, the present invention is directed to a method of treating an IFN Excess Disorder in a subject, which comprises administering to the subject a MORC3 therapeutic agent. In some embodiments, the interferon disorder is an IFNB1 Disorder. In some embodiments, the present invention is directed to a method of treating an IFN Excess Disorder in a subject, which comprises increasing the amount of MORC3 in the subject. In some embodiments, the subject is in need thereof. In some embodiments, the IFN Excess Disorder is an autoimmune or inflammatory disease. In some embodiments, the IFN Excess Disorder is rheumatoid arthritis, psoriasis, vitiligo, hypothyroidism, hyperthyroidism, idiopathic thrombocytopenic purpura, autoimmune hemolytic anemia, myasthenia gravis, Addison disease, celiac disease, polymyositis, or superimposed autoimmune hepatitis. In some embodiments, the autoimmune or inflammatory disease is multiple sclerosis. In some embodiments, the multiple sclerosis is primary progressive multiple sclerosis or relapsing-remitting multiple sclerosis. In some embodiments, the IFN Excess Disorder is an IFNB1 Disorder. In some embodiments, the MORC3 therapeutic agent is a MORC3 protein. In some embodiments, the MORC3 therapeutic agent is a MORC3 activator. In some embodiments, the MORC3 therapeutic agent stabilizes expression of MORC3. In some embodiments, the MORC3 protein comprises a sequence that has at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 1. In some embodiments, the method further comprises inhibiting or reducing the activity of MRE in the subject. In some embodiments, the sequence of the MRE in the subject at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 3.

In some embodiments, the present invention is directed to a method of treating IFN Deficiency Disorder in a subject, which comprises administering to the subject a MORC3 therapeutic agent. In some embodiments, the present invention is directed to a method of treating IFN Deficiency Disorder in a subject, which comprises decreasing the amount of MORC3 in the subject. In some embodiments, the IFN Deficiency Disorder is a viral infection. In some embodiments, the viral infection is caused by a herpes virus, a hepatitis virus, or a coronavirus. In some embodiments, the IFN Deficiency Disorder is a cancer. In some embodiments, the cancer is a leukemia, a lymphoma, a melanoma, a sarcoma, or an adenocarcinoma. In some embodiments, the subject is in need thereof. In some embodiments, the IFN Deficiency Disorder is a viral infection such as that caused by a herpes virus, a hepatitis virus, or a coronavirus. In some embodiments, the IFN Deficiency Disorder is a cancer such as a leukemia, a lymphoma, a melanoma, a sarcoma, or an adenocarcinoma. In some embodiments, the cancer is colon cancer. In some embodiments, the IFN Deficiency Disorder is an IFNB1 Disorder. In some embodiments, the MORC3 therapeutic agent is a MORC3 inhibitor such as an siRNA, an ATPase inhibitor (e.g., a small molecule ATPase inhibitor), or an ICP0 protein. In some embodiments, the MORC3 therapeutic agent is a MORC3 inhibitor such as an siRNA, an ATPase inhibitor (e.g., a small molecule ATPase inhibitor), or an ICP0 protein. In some embodiments, the ICP0 protein comprises a sequence that has at least about 95%, at least about 96%, at least about 97%, at least about 98%, at least about 99%, or 100% sequence identity to SEQ ID NO: 2.

In some embodiments, the present invention is directed to a method of treating an interferon disorder (alternatively referred to herein as a “MORC3-modulated disease” as it is modulated by MORC3) in a subject, which comprises administering to the subject a MORC3 therapeutic agent. In some embodiments, the MORC3 therapeutic agent is an siRNA, an ATPase inhibitor (e.g., a small molecule ATPase inhibitor), a MORC3 protein, or an ICP0 protein. In some embodiments, the method comprises decreasing the amount of endogenous interferon in the subject by administering the MORC3 therapeutic agent, wherein the MORC3 therapeutic agent is a MORC3 activator. In some embodiments, the method comprises decreasing the amount of endogenous interferon in the subject by administering the MORC3 therapeutic agent, which is a MORC3 protein, and/or stabilizes the expression of MORC3. In some embodiments, the method comprises increasing the amount of endogenous interferon in the subject by administering the MORC3 therapeutic agent, wherein the MORC3 therapeutic agent is a MORC3 inhibitor. In some embodiments, the method comprises increasing the amount of endogenous interferon in the subject by administering the MORC3 therapeutic agent, which is an siRNA, an ATPase inhibitor (e.g., a small molecule ATPase inhibitor), and/or an ICP0 protein. In some embodiments, the MORC3 protein comprises a sequence that has at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 1. In some embodiments, the ICP0 protein comprises a sequence that has at least about 95%, at least about 96%, at least about 97%, at least about 98%, at least about 99%, or 100% sequence identity to SEQ ID NO: 2. In some embodiments, the subject is in need thereof.

In some embodiments, the present invention is directed to a method of treating an interferon disorder (alternatively referred to herein as a “MORC3-modulated disease” as it is modulated by MORC3) in a subject, which comprises administering to the subject a MORC3 therapeutic agent. In some embodiments, the MORC3-modulated disease is an autoimmune or inflammatory disease. In some embodiments, the autoimmune or inflammatory disease is multiple sclerosis. In some embodiments, the multiple sclerosis is primary progressive multiple sclerosis or relapsing-remitting multiple sclerosis. In some embodiments, the MORC3-modulated disease is rheumatoid arthritis, psoriasis, vitiligo, hypothyroidism, hyperthyroidism, idiopathic thrombocytopenic purpura, autoimmune hemolytic anemia, myasthenia gravis, Addison disease, celiac disease, polymyositis, or superimposed autoimmune hepatitis. In some embodiments, the MORC3 therapeutic agent is a MORC3 protein. In some embodiments, the MORC3 protein comprises a sequence that has at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 1. In some embodiments, the MORC3 therapeutic agent is a MORC3 activator and/or stabilizes the expression of MORC3. In some embodiments, the subject is in need thereof.

In some embodiments, the present invention is directed to a method of treating an interferon disorder (alternatively referred to herein as a “MORC3-modulated disease” as it is modulated by MORC3) in a subject, which comprises administering to the subject a MORC3 therapeutic agent. In some embodiments, the MORC3-modulated disease is cancer. In some embodiments, the cancer is a leukemia, a lymphoma, a melanoma, a sarcoma, or an adenocarcinoma. In some embodiments, the cancer is colon cancer. In some embodiments, the MORC3-modulated disease is a viral infection. In some embodiments, the viral infection is caused by a herpes virus, a hepatitis virus, or a coronavirus. In some embodiments, the MORC3 therapeutic agent is a MORC3 inhibitor such as an siRNA, an ATPase inhibitor (e.g., a small molecule ATPase inhibitor), or an ICP0 protein. In some embodiments, the ICP0 protein comprises a sequence that has at least about 95%, at least about 96%, at least about 97%, at least about 98%, at least about 99%, or 100% sequence identity to SEQ ID NO: 2. In some embodiments, the subject is in need thereof.

In some embodiments, the present invention is directed to an assay method for determining whether a candidate compound is a MORC3 inhibitor, which comprises contacting the candidate compound with a monocyte and measuring any interferon response induced thereby in the monocyte. In some embodiments, the assay method further comprises contacting the candidate compound with a genetically modified monocyte that deficient in MORC3 activity, measuring any interferon response induced thereby in the genetically modified monocyte, and comparing the interferon response in the monocyte to the interferon response in the genetically modified cell.

In some embodiments, the present invention is directed to a method of increasing interferon expression by a cell, which comprises (a) the decreasing amount of MORC3 in the cell, (b) increasing MRE activity in the cell, or both (a) and (b). In some embodiments, the MORC3 is decreased by administering an ICP0 protein to the cell or contacting the MORC3 with an ATPase inhibitor (e.g., a small molecule ATPase inhibitor), or inactivating the MORC3 gene in the cell using recombinant techniques, or silencing the MORC3 gene with an siRNA. In some embodiments, the ICP0 protein comprises a sequence that has at least about 95%, at least about 96%, at least about 97%, at least about 98%, at least about 99%, or 100% sequence identity to SEQ ID NO: 2.

A method of decreasing interferon expression by a cell, which comprises (a) increasing the amount of a MORC3 protein in the cell, (b) reducing MRE activity in the cell, or (c) both (a) and (b). In some embodiments, the amount of MORC3 is increased by administering a MORC3 protein to the cell and/or increasing the expression of a MORC3 protein in the cell using recombinant techniques. In some embodiments, the MORC3 protein comprises a sequence that has at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 1. In some embodiments, the MRE activity is reduced by recombinant techniques, e.g., modifying or deleting the MRE sequence in the cell.

In some embodiments, the present invention provides a MORC3 protein for use as a medicament. In some embodiments, the present invention provides a MORC3 protein for use in the treatment of an interferon disorder in a subject. In some embodiments, the interferon disorder is an IFN Deficiency Disorder. In some embodiments, the IFN Deficiency Disorder is a viral infection. In some embodiments, the viral infection is caused by a herpes virus, a hepatitis virus, or a coronavirus. In some embodiments, the IFN Deficiency Disorder is a cancer. In some embodiments, the cancer is a leukemia, a lymphoma, a melanoma, a sarcoma, or an adenocarcinoma. In some embodiments, the IFN Deficiency Disorder is a viral infection such as that caused by a herpes virus, a hepatitis virus, or a coronavirus. In some embodiments, the IFN Deficiency Disorder is a cancer such as a leukemia, a lymphoma, a melanoma, a sarcoma, or an adenocarcinoma. In some embodiments, the cancer is colon cancer. In some embodiments, the MORC3 protein comprises a sequence that has at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 1.

In some embodiments, the present invention provides an ICP0 protein for use as a medicament. In some embodiments, the present invention provides an ICP0 protein for use in the treatment of an interferon disorder in a subject. In some embodiments, the interferon disorder is an IFN Excess Disorder. In some embodiments, the IFN Excess Disorder is rheumatoid arthritis, psoriasis, vitiligo, hypothyroidism, hyperthyroidism, idiopathic thrombocytopenic purpura, autoimmune hemolytic anemia, myasthenia gravis, Addison disease, celiac disease, polymyositis, or superimposed autoimmune hepatitis. In some embodiments, the autoimmune or inflammatory disease is multiple sclerosis. In some embodiments, the multiple sclerosis is primary progressive multiple sclerosis or relapsing-remitting multiple sclerosis. In some embodiments, the ICP0 protein comprises a sequence that has at least about 95%, at least about 96%, at least about 97%, at least about 98%, at least about 99%, or 100% sequence identity to SEQ ID NO: 2.

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description explain the principles of the invention.

### DESCRIPTION OF THE DRAWINGS

This invention is further understood by reference to the drawings wherein:

FIG. 1-a-FIG. 1-f: Virulence factor-triggered immune sensing. (FIG. 1-a, FIG. 1-b) BLaER1 monocytes of the indicated genotypes were infected with HSV-1 for 24 h or transfected with Salmon Sperm DNA for 3 h. Gene-expression of one representative clone of two per genotype (except WT) is depicted as mean±SEM of three independent experiments. MOI, multiplicity of infection. * p<0.05; ** p<0.01; ns=not significantly different than WT, tested by one-way ANOVA and Dunnett's post hoc test. (FIG. 1-c) Redundancy in immune sensing of HSV-1 between the DNA-STING pathway and detection of ICP0. (FIG. 1-d-FIG. 1-f) BLaER1 monocytes expressing doxycycline-inducible virulence factors were stimulated with doxycycline for 24 h. Gene-expression as quantified by q-RT-PCR is depicted as mean±SEM of three independent experiments (FIG. 1-d). Transcriptomic changes were profiled by RNA-seq in three independent experiments and were analyzed by principal component analysis (PCA) of variance stabilizing transformed counts (FIG. 1-e) and heatmap of log normalized counts of the top 40 most variable genes (column normalized) (FIG. 1-f). ND=not detected.

FIG. 2-a-FIG. 2-g: MORC3 is a novel negative regulator of IFN. (FIG. 2-a) Hypothesized mechanism in which ICP0 degrades a negative regulator of IFN. (FIG. 2-b) Schematic of genome wide CRISPR screen to identify negative regulator of IFN. (FIG. 2-c) Cas9-expressing BLaER1 cells were transduced with individual sgRNAs targeting and Viperin expression was analyzed by FACS. Mean+SEM of n=4. * p<0.05; ** p<0.01; ns=not significantly different than scramble sgRNA, tested by one-way ANOVA and Dunnett's post hoc test. (FIG. 2-d) IFNAR1^{-/-}IFNAR2^{-/-}STING^{-/-}SP 100^{-/-} BLaER1 monocytes (lacking factors that would otherwise restrict ΔICP0 mutant virus) were infected with HSV-1 for 3 h. One representative immunoblot of two is shown. (FIG. 2-e) Gene expression of BLaER1 monocytes is shown as mean±SEM of n=2-3 from one representative clone or two (multiple KOs) or one clone (WT and MORC3^{-/-}). ** p<0.01; ns=not significantly different than WT, tested by two-way ANOVA and Dunnett's post hoc test. (FIG. 2-f, FIG. 2-g) Transcriptional changes in BLaER1 monocytes as detected by RNA-seq in three independent experiments are depicted by PCA analysis (FIG. 2-f) and heatmap (FIG. 2-g). These data are partially duplicated from FIG. 1-e.

FIG. 3-a-FIG. 3-h: Dual functions of MORC3 allows self-guarding. (FIG. 3-a) The titer of HSV-1 stocks at 2.5×10⁵ U2OS-FFU/ml was determined on HCT116-Cas9 cells that were transduced with the indicated sgRNAs. Mean±SEM of n=6. * p<0.05; ** p<0.01; *** p<0.001; ns=not significantly different, tested by paired, two-sided t-test. (FIG. 3-b) BLaER1 monocytes were infected with HSV-1 and viral progeny were quantified after 48 h. Mean (line) and individual values of three independent experiments. LOD=limit of detection. * p<0.05; ns=not significantly different than the corresponding MORC3 sufficient condition, tested by one-way ANOVA and Dunnett's post hoc test. (FIG. 3-c) Two proposed functions of MORC3 that allow self-guarding. (FIG. 3-d, FIG. 3-e) Transcriptomic changes in BLaER1 monocytes as detected by RNA-seq in three independent experiments: heatmap of log normalized counts of the top 40 most variable genes (column normalized) (FIG. 3-d) or PCA of variance stabilizing transformed counts (FIG. 3-e). Data in (FIG. 3-e) is partially duplicated from FIG. 2-f (FIG. 3-f) Log transcripts per million (TPM) of genes in BLaER1 monocytes of genes clustered near IFNB1 on chromosome 9 as detected by RNA-seq. Depicted are means of three independent experiments. All protein coding genes within this region are depicted. (FIG. 3-g, FIG. 3-h) STAT1^{-/-}STAT2^{-/-}BLaER1-Cas9 expressing a randomly integrated IFNB1-promoter-Luciferase reporter were transduced with the indicated sgRNAs and stimulated with cytosolic DNA for 24 h. Luciferase signal and IFNβ secretion is depicted as mean±SEM of n=3. p<0.05; ns=not significantly different, tested by paired, two-sided t-test.

FIG. 4-a-FIG. 4-g: Positional repression of IFNB1 explains IFN de-repression in MORC3 deficient cells. (FIG. 4-a) BLaER1 monocytes of the indicated genotypes were transfected with DNA for 12 h. Gene-expression as measured by q-RT-PCR of one representative clone of two per genotype is depicted as mean±SEM of four independent experiments. (FIG. 4-b) Log transcripts per million (TPM) of IFN genes in BLaER1 monocytes were detected by RNAseq in three independent experiments. (FIG. 4-c) Gene expression as measured by q-RT-PCR of BLaER1 monocytes of the indicated genotypes is shown as mean±SEM of n=3 from one representative clone of two (multiple KOs) or one clone (WT and MORC3^{-/-}). (FIG. 4-d-FIG. 4-f) Transcriptomic changes in BLaER1 monocytes as detected by RNA-seq in three independent experiments are depicted by heatmap analysis (FIG. 4-d) and PCA (FIG. 4-e, FIG. 4-f). The distance on PC1 between samples and the mean of mCherry-expressing cells was calculated from data in FIG. 9-c and FIG. 9-f and is depicted from three independent experiments. *** p<0.001; ns=not significantly different than WT, tested by two-way ANOVA and Dunnett's post hoc test. (FIG. 4-g) Overview of virulence-factor-triggered immune signaling.

FIG. 5-a-FIG. 5-c: Redundancy between DNA- and ICP0-mediated sensing of HSV-1 in BLaER1 monocytes. BLaER1 monocytes were infected with HSV-1 for 24 h or transfected with DNA for 3 h. Gene-expression analysis or viral titer in supernatant of one representative clone of two per genotype (except WT) is depicted as mean±SEM of three independent experiments. * p<0.05; ** p<0.01; ns=not significantly different than WT, tested by two-way ANOVA and Dunnett's post hoc test. LOD=limit of detection.

FIG. 6-a-FIG. 6-b: Redundancy between DNA- and ICP0-mediated sensing of HSV-1 in THP1 and U937 cells. PMA-differentiated THP1 or U937 human myeloid-like cells were infected with HSV-1 for 24 h or transfected with 2′-3′-cGAMP for 4 h. Gene-expression analysis or viral titer in supernatant is depicted as mean±SEM of three independent experiments. ND=not detected. * p<0.05; ** p<0.01; *** p<0.001; ns=not significantly different than WT, tested by two-way ANOVA and Dunnett's post hoc test.

FIG. 7-a-FIG. 7-c: Virulence factors induce IFN in monocytes independently of PRR-signaling hubs. BLaER1 monocytes, PMA-differentiated THP1 or U937 cells expressing doxycycline-inducible virulence factors were stimulated with doxycycline for 24 h. Gene-expression as quantified by q-RT-PCR is depicted as mean±SEM of three independent experiments. IFNB1 expression levels in FIG. 7-a are partially duplicated from FIG. 1-d. * p<0.05; ** p<0.01; *** p<0.001; ns=not significantly different than the corresponding mCherry-expressing condition, tested by two-way ANOVA and Dunnett's post hoc test.

FIG. 8-a-FIG. 8-d: Virulence factor activity is required for IFN induction. (FIG. 8-a, FIG. 8-b) BLaER1 monocytes expressing doxycycline-inducible virulence factors were stimulated with doxycycline for 24 h. Gene-expression as quantified by q-RT-PCR is depicted as mean±SEM of three independent experiments and protein expression by immunoblot is depicted from one representative experiment of two. *** p<0.001; ns=not significantly different than the corresponding mCherry-expressing condition, tested by two-way ANOVA and Dunnett's post hoc test. (FIG. 8-c) Transcriptomic changes in BLaER1 monocytes as detected by RNA-seq were analyzed by PCA of variance stabilizing transformed counts. Top 10 genes that contribute to individual PC directions are depicted. (FIG. 8-d) Modules from the Molecular Signatures Database that were found to be enriched in differentially expressed genes upon virulence factor expression in BLaER1 monocytes. Background gene set includes all genes that have base mean expression of at least 1 in the virulence factor expressing condition.

FIG. 9-a-FIG. 9-h: Validation of MORC3 as a repressor of IFN. (FIG. 9-a) Cell sorting strategy for the genome-wide CRISPR screen to identify negative regulators of IFN. (FIG. 9-b) U2OS cells were infected with HSV-1 for 24 h. One representative immunoblot of two is shown. (FIG. 9-c, FIG. 9-d) Gene expression of BLaER1 monocytes is shown as mean±SEM of n=2-3 from one representative clone or two (multiple KOs) or one clone (WT and MORC3^{-/-}). (FIG. 9-e-FIG. 9-g) Immunoblot of BLaER1 monocytes of indicated genotypes. (FIG. 9-h) Modules from the Molecular Signatures Database that were found to be enriched in differentially expressed genes in MORC3^{-/-} BLaER1 monocytes. Background gene set includes all genes that have base mean expression of at least 1 in MORC3^{-/-} BLaER1 monocytes.

FIG. 10-a-FIG. 10-f: Characterization of MORC3 deficiency in THP1, U937 and HCT116 cells. Gene-expression in PMA-differentiated THP1-Cas9 or PMA-differentiated U937-Cas9 human myeloid-like cells or HCT116-Cas9 cells expressing indicated sgRNAs is depicted as mean±SEM of three independent experiments. Protein expression in the same cells was analyzed by immunoblot. ND=not detected. *** p<0.001; ns=not significantly different than the corresponding scramble sgRNA-expressing condition, tested by two-way ANOVA and Dunnett's post hoc test.

FIG. 11-a-FIG. 11-b: MORC3 deficiency leads to de-repression of a gene cluster at chromosome 9. (FIG. 11-a) Adjusted p-values of differentially expressed genes between MORC3^{-/-}IFNAR1^{-/-}IFNAR2^{-/-} and IFNAR1^{-/-}IFNAR2^{-/-} mCherry expressing cells are depicted. Data point sizes represent normalized effect size, calculated as the effect size multiplied by the normalized mean counts of all MORC3^{-/-}IFNAR1^{-/-}IFNAR2^{-/-} and IFNAR1^{-/-}IFNAR2^{-/-} expressing cells. (FIG. 11-b) BLaER1 monocytes were stimulated with indicated PAMPs for 3 h or 24 h. Gene-expression analysis is depicted as mean±SEM of five independent experiments.

FIG. 12-a: Quantification of expression of ERV families. ERV family expression was quantified with RepEnrich2 in IFNAR1^{-/-}IFNAR2^{-/-} mCherry vs MORC3^{-/-}IFNAR1^{-/-}IFNAR2^{-/-} monocytes. ERVs with an FDR<0.05 are highlighted in red. The recommended RepEnrich2-EdgeR pipeline did not detect any regulation of ERVs upon MORC3 deficiency. RepEnrich2-DeSeq2 analysis suggested minimal up- and down-regulation of ERV families.

FIG. 13-a-FIG. 13-f: Positional de-repression of IFNB1 explains IFN induction by virulence factors. (FIG. 13-a) BLaER1 monocytes were transfected with DNA for 12 h. Cytokine secretion from one representative clone of two per genotype is depicted as mean±SEM of three independent experiments. (FIG. 13-b) Mean log transcripts per million (TPM) of a gene cluster at chromosome 9 in BLaER1 monocytes. Mean TPM is calculated for each condition across three experiments. All protein coding genes within this region are depicted. (FIG. 13-c) PCA of variance stabilizing transformed gene counts in BLaER1 monocytes in three independent experiments. Data is partially duplicated from FIG. 3-e. (FIG. 13-d) Log transcripts per million (TPM) of IFN genes in BLaER1 monocytes upon virulence factor expression were detected by RNAseq in three independent experiments. (FIG. 13-e) BLaER1 monocytes expressing doxycycline-inducible virulence factors were stimulated with doxycycline for 24 h. Gene expression is shown as mean±SEM of n=3 from one representative clone of two. (FIG. 13-f) BLaER1 monocytes expressing doxycycline-inducible virulence factors were stimulated with doxycycline for 24 h. Transcriptomic changes as detected by RNA-seq in three independent experiments were analyzed PCA. Data is partially duplicated from FIG. 1-f.

FIG. 14-a-FIG. 14-j: A MORC3-regulated DNA element explains positional IFNB1 activation. (FIG. 14-a) Adjusted p-values of increased accessibility in MORC3^{-/-}IFNAR1^{-/-}IFNAR2^{-/-} vs IFNAR1^{-/-}IFNAR2^{-/-} BLaER1 monocytes of non-promoter-associated ATAC-seq peaks from three independent experiments. Peaks are labeled according to the adjacent gene and peaks overlapping with the MRE are highlighted in red. (FIG. 14-b) A MORC3-regulated DNA element located in an intron of FOCAD. Sum of normalized ATAC-seq reads from three independent experiments is depicted. Sequencing overlapping a MREA allele is indicated. (FIG. 14-c) STAT1^{-/-}STAT2^{-/-} Cas9 (“WT”) or STAT1^{-/-}STAT2^{-/-} MRE^{Δ/Δ} Cas9 BLaER1 monocytes of indicated genotype were stimulated with PAMPs for 3 h or left untreated. Gene expression is shown as mean+SEM of three independent experiments. (FIG. 14-d) Schematic overview of tracking allele-specific gene expression. (FIG. 14-e) Allele-specific gene expression STAT1^{-/-}STAT2^{-/-} Cas9 or STAT1^{-/-}STAT2^{-/-} MRE^{+/Δ} Cas9 cells that were transduced with indicated sgRNAs and stimulated with DNA for 3 h if indicated. Data is depicted as mean+SEM of three independent clones (harboring different IFNB1 indels) as average from three experiments. (FIG. 14-f) Transcriptional changes in BLaER1 monocytes as detected by RNA-seq in two independent experiments are depicted by heatmap (column normalized). High-confidence MORC3 targets (genes de-repressed in MORC3^{-/-}IFNAR1^{-/-}IFNAR2^{-/-}, MORC3^{-/-} IFNB1^{-/-} and MORC3^{-/-}STAT1^{-/-}STAT2^{-/-} Cas9) cells are depicted. (FIG. 14-g, FIG. 14-h) STAT1^{-/-}STAT2^{-/-} Cas9 (“WT”) or STAT1^{-/-}STAT2^{-/-} MRE^{Δ/Δ} Cas9 BLaER1 monocytes expressing doxycycline-inducible virulence factors were stimulated with doxycycline for 24 h. Gene expression is shown as mean+SEM of four independent experiments. (FIG. 14-i) STAT1^{-/-}STAT2^{-/-} Cas9 (“WT”) or STAT1^{-/-}STAT2^{-/-} MRE^{Δ/Δ} Cas9 BLaER1 were infected with HSV-1 at indicated MOI for 4-5 h. Gene expression is shown as mean+SEM of five independent experiments. (FIG. 14-j) Overview of the self-guarded MORC3 pathway that detects virulence factors from DNA viruses via genomic location-dependent de-repression of IFNB1. *** p<0.001; ns=not significantly different than WT two-way ANOVA and Bonferroni's post hoc test. See Source Data for exact p values.

FIG. 15-a-FIG. 15-h: Validation of the MORC3-regulated DNA element. (FIG. 15-a) Protein expression of indicated BLaER1 monocytes was analyzed by immunoblot. (FIG. 15-b) Consensus sequences of amplicon sequencing at the MRE locus from STAT1^{-/-}STAT2^{-/-} MRE^{Δ/Δ} BLaER1 Cas9 cells were aligned to the WT reference. 3166 bp were omitted from the reference sequence. (FIG. 15-c) Protein expression of indicated BLaER1 monocytes was analyzed by immunoblot. (FIG. 15-d) Gene expression of indicated BLaER1 monocytes is depicted as mean±SEM of three independent experiments from one representative clone of two (MORC3^{-/-} MLLT3^{-/-} IFNAR1^{-/-} IFNAR2^{-/-} and MORC3^{-/-} FOCAD^{-/-} IFNAR1^{-/-} IFNAR2^{-/-}) or one (IFNAR1^{-/-}IFNAR2^{-/-} and MORC3^{-/-}IFNAR1^{-/-}IFNAR2^{-/-}) per genotype. ns=not significantly different than the IFNAR1^{-/-}IFNAR2^{-/-} condition, tested by two-way ANOVA and Dunnett's post hoc test. From top to bottom, the sequences are SEQ ID NO: 59+SEQ ID NO: 60, SEQ ID NO: 61, and SEQ ID NO: 62. (FIG. 15-e) Protein expression of indicated BLaER1 monocytes was analyzed by immunoblot. (FIG. 15-f) Log transcripts per million (TPM) of high-confidence MORC3 targets (genes de-repressed in MORC3^{-/-}IFNAR1^{-/-}IFNAR2^{-/-}, MORC3^{-/-}IFNB1^{-/-} and MORC3^{-/-} STAT1^{-/-}STAT2^{-/-} Cas9) in WT=MORC3^{-/-}STAT1^{-/-}STAT2^{-/-} Cas9 or indicated genotypes are depicted as mean±SEM of two independent RNA-seq experiments. (FIG. 15-g) STAT1^{-/-}STAT2^{-/-} Cas9 (“WT”) or STAT1^{-/-}STAT2^{-/-} MRE^{Δ/Δ} BLaER1 Cas9 were infected with HSV-1 at indicated MOI for 4-5 h. One representative immunoblot of two is depicted. (FIG. 15-h) Peripheral blood human monocytes were nucleofected with indicated sgRNA:Cas9 complexes and differentiated with M-CSF for 5 days. Gene expression from three independent donors is depicted as mean±SEM. * p<0.05; ** p<0.01; *** p<0.001; significantly different than the scramble sgRNA condition, tested by two-way ANOVA and Bonferroni's post hoc test. ND=not detected.

FIG. 16-a-FIG. 16-c: Targeting of Morc3 in vivo results in interferon induction and tumor clearance. (FIG. 16-a) Mice harboring a floxed Morc3 gene were crossed to mice harboring a tamoxifen-inducible Cre (CreERT2). These mice were fed chow containing tamoxifen for 10 days. This results in systemic deletion of the Morc3 gene in the CreERT2+ mice (red symbols) but not in control mice (blue symbols) that are lacking Cre. After 11 additional days (during which the mice were fed normal chow), spleens were harvested and interferon (Ifnb) induction was assessed by quantitative reverse-transcriptase PCR. Mice in which Morc3 is deleted exhibit about a 100-fold induction of Ifnb. (FIG. 16-b) The Morc3-floxed CreERT2 mice described in FIG. 16-a were implanted with 500,000 MC38-Luc tumor cells on day 1 and fed tamoxifen-containing chow to delete Morc3 from days 1-10. Mice harboring CreERT2 (green symbols) that delete Morc3 exhibit enhanced tumor control whereas control (CreERT2-negative) mice (red symbols) that retain Morc3 expression exhibit normal tumor growth. (FIG. 16-c) The mice described in FIG. 16-a were implanted with 500,000 MC38-Luc tumor cells, as in FIG. 16-b, but instead of oral provision of tamoxifen in the chow, tamoxifen was injected intraperitoneally once per day on days 1-5. Mice harboring CreERT2 (green symbols) that delete Morc3 exhibit enhanced tumor control whereas control (CreERT2-negative) mice (red symbols) that retain Morc3 expression exhibit normal tumor growth.

### DETAILED DESCRIPTION OF THE INVENTION

Described herein is a unique ‘self-guarded’ immune pathway in human monocytes, in which guarding and guarded function are united in one protein, MORC family CW-type zinc finger protein 3 (MORC3). Human MORC3 has Accession No. NP_056173.1 (SEQ ID NO: 1). This self-guarded immune pathway is triggered by a variety external pathogenic factors, e.g., ICP0, which is a key virulence factor of Herpes Simplex Virus-1 (HSV-1). ICP0 (Accession No. YP_009137074.1, SEQ ID NO: 2) results in robust induction of anti-viral type I interferon (IFN). Surprisingly, induction of IFN by ICP0 is independent of canonical immune pathways and the IRF3/7 transcription factors. A CRISPR-screen identified the ICP0 target MORC3 as an important negative regulator of IFN. Loss of MORC3 recapitulates the IRF3/7-independent IFN response induced by ICP0. Mechanistically, ICP0 degrades MORC3, which leads to de-repression of the IFNB1 locus in a genomic location-specific manner to drive the anti-viral IFN response. Besides repressing IFNB1, MORC3 is also a direct restriction factor of HSV-1. The results herein suggest a model in which the primary anti-viral function of MORC3 is ‘self-guarded’ by its secondary IFN-repressing function: thus, a virus that degrades MORC3 to avoid its primary anti-viral function will unleash the secondary anti-viral IFN response. Thus, a self-guarded bi-functional pathways such as the MORC3 pathway might be a central feature of animal immunity.

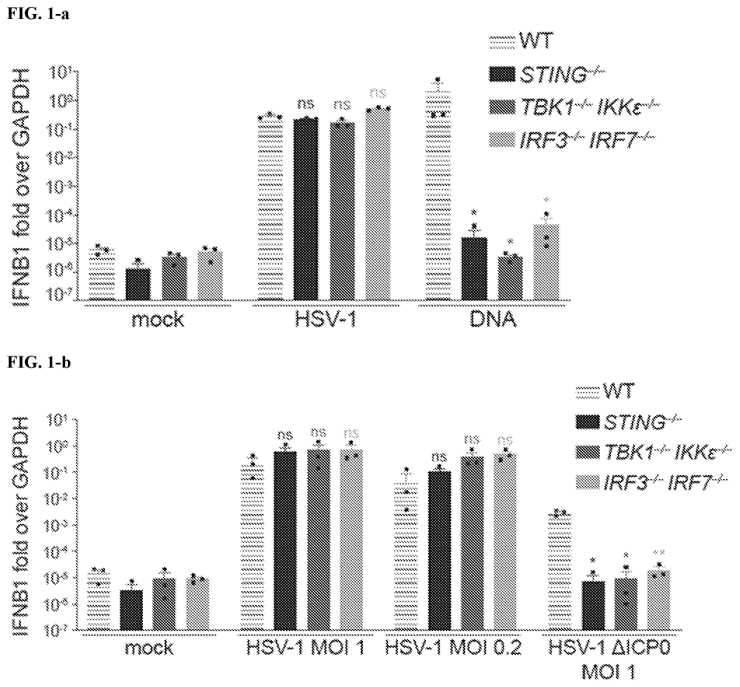

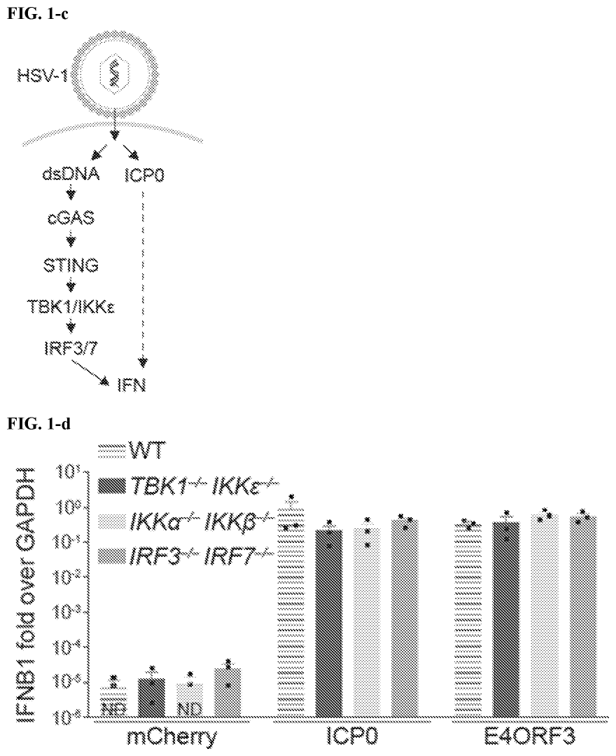

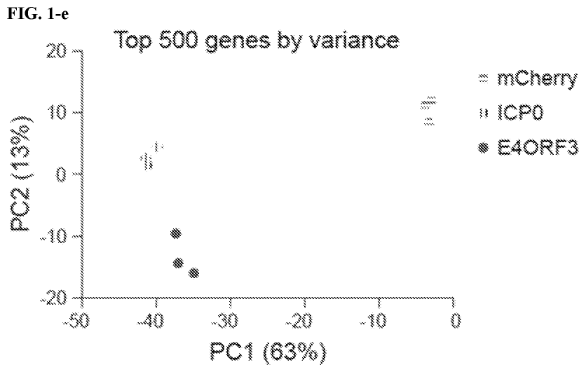

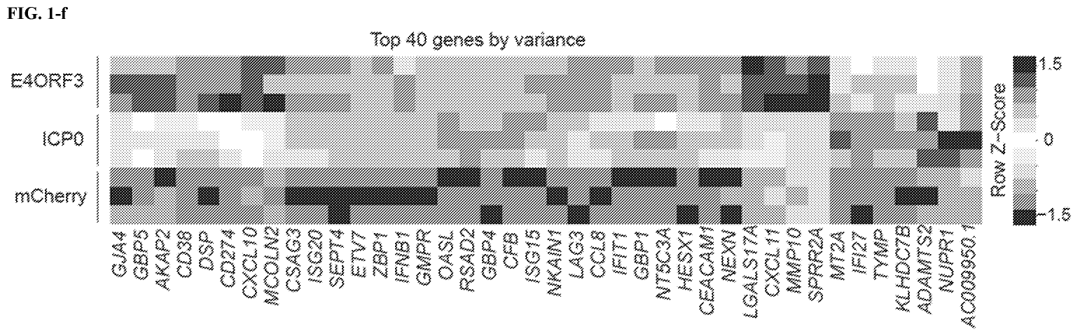

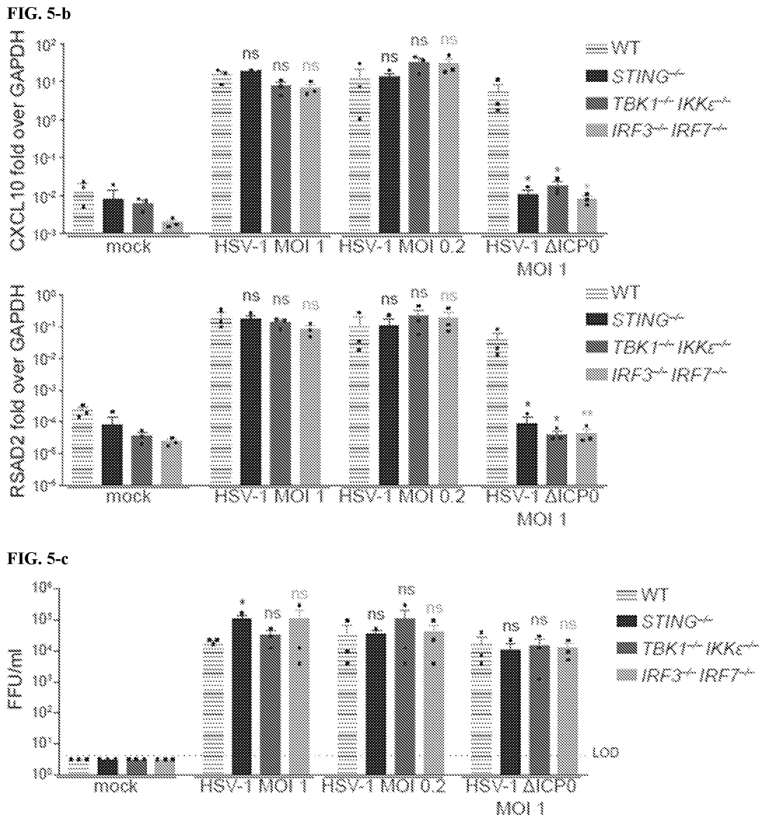

To study the innate immune response to herpes viruses, human BLaER1 monocytes were infected with HSV-1. Surprisingly, the induction of IFN as measured by transcription of IFNB1 and the ISGs RSAD2 and CXCL10 was not solely dependent on the STING pathway of viral DNA recognition (FIG. 1-a, FIG. 5-a). To ask if other PRRs were involved in the STING-independent IFN response, the central signaling hubs TBK1/IKKε and IRF3/7, which are important for canonical PRR-dependent pathways of IFN induction, including the recently described STING-independent DNA-sensing pathway (SIDSP) were targeted. IFN induction during HSV-1 infection did not rely on TBK1/IKKε and IRF3/7 (FIG. 1-a, FIG. 5-a). Thus, IFN induction by HSV-1 can occur independently of canonical PRRs. In contrast, IFN induction in response to transfected dsDNA depended entirely on the STING pathway and downstream TBK1/IKKε and IRF3/7 signaling components (FIG. 1-a, FIG. 5-a).

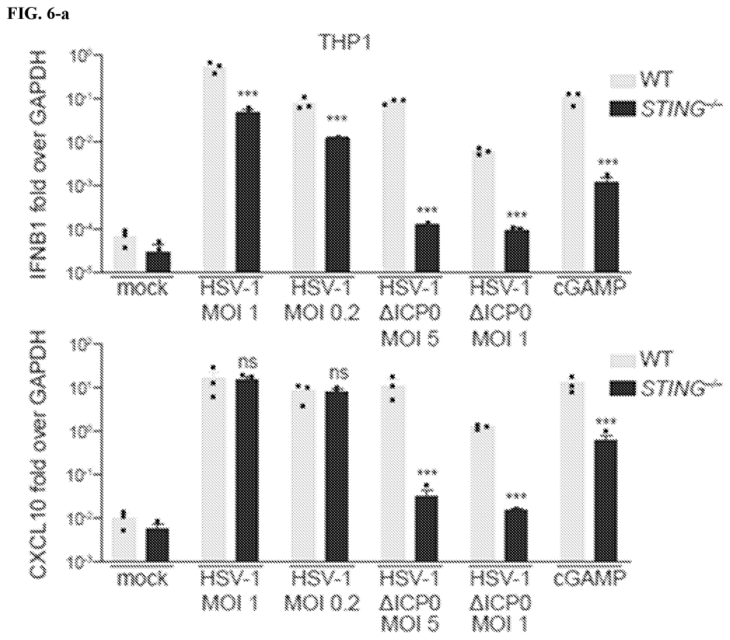

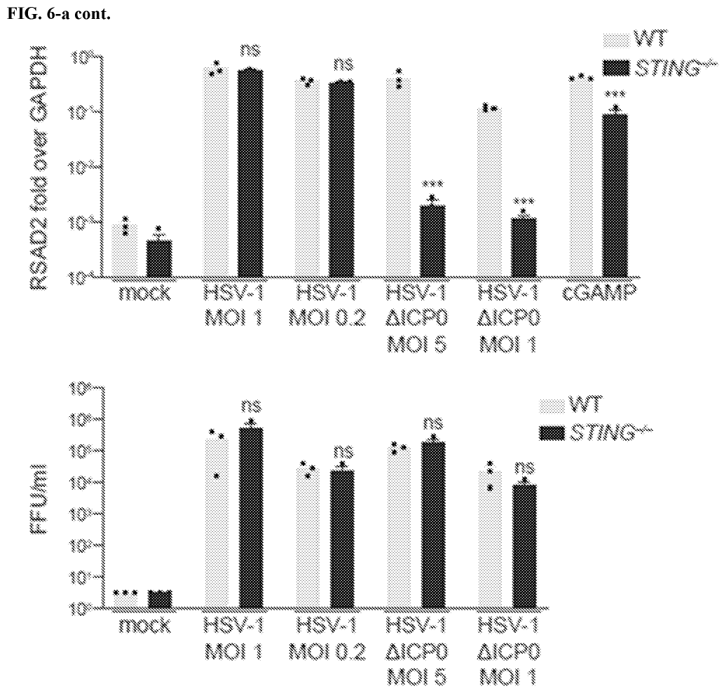

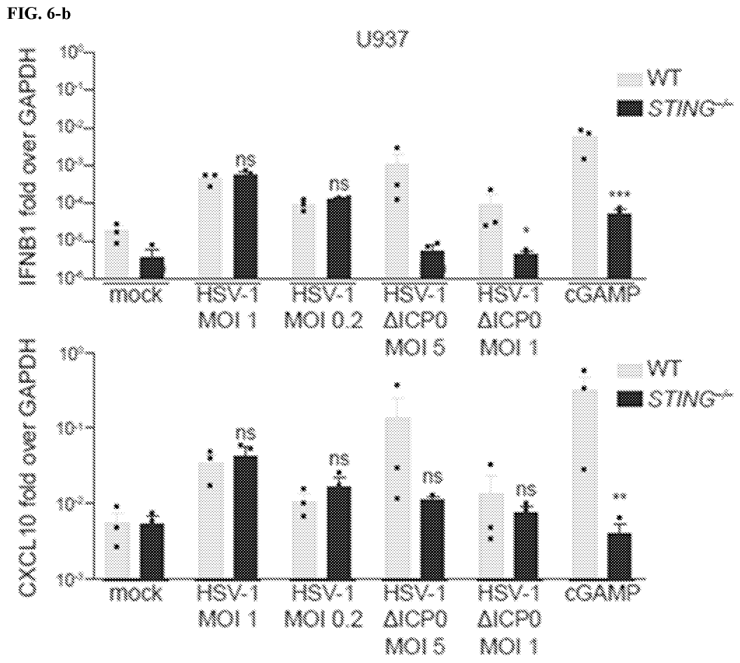

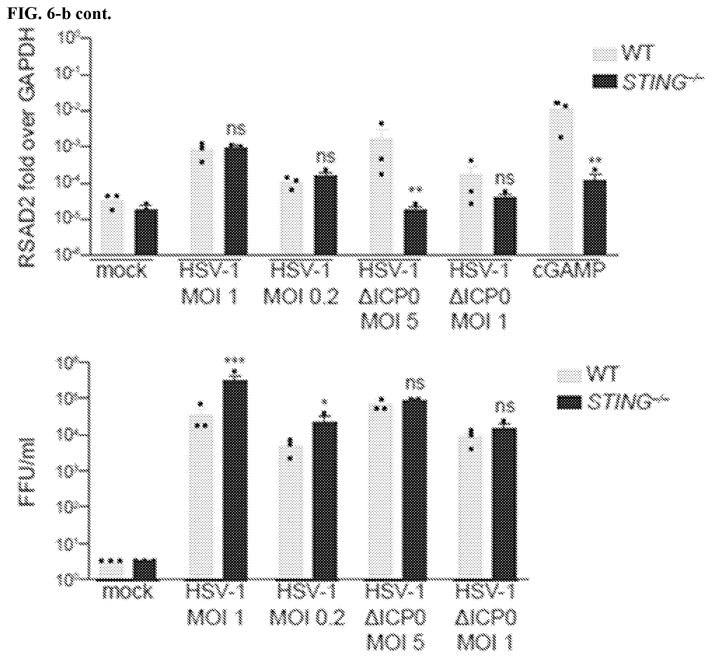

These results suggest that an additional PRR-independent innate immune pathway detects HSV-1 infection in human monocytes. The possibility that the immune system is activated by a viral virulence factor was considered. A key virulence factor of HSV-1 is the SUMO-directed E3 ubiquitin ligase ICP0, which facilitates lytic replication by recognizing sumoylated ND10 nuclear body proteins, targeting them for proteasomal degradation. ICP0 was necessary for induction of PRR-independent IFN in BLaER1 monocytes (FIG. 1-b, FIG. 5-b) while loss of ICP0 only modestly affected HSV-1 replication (FIG. 5-c). Induction of IFN by HSV-1 was abrogated only by deleting both the STING pathway from host cells and ICP0 from HSV-1 (FIG. 1-b). Thus, during HSV-1 infection of BLaER1 monocytes, two redundant pathways induce IFN: STING-mediated viral DNA-sensing and another innate sensing pathway, which requires ICP0 (FIG. 1-c). This redundancy was confirmed in THP1 and U937 cells, two other human monocyte-like cell culture models. As in BLaER1 monocytes, IFN was induced by either host STING or viral ICP0 and was abrogated only in absence of both (FIG. 6-a, FIG. 6-b).

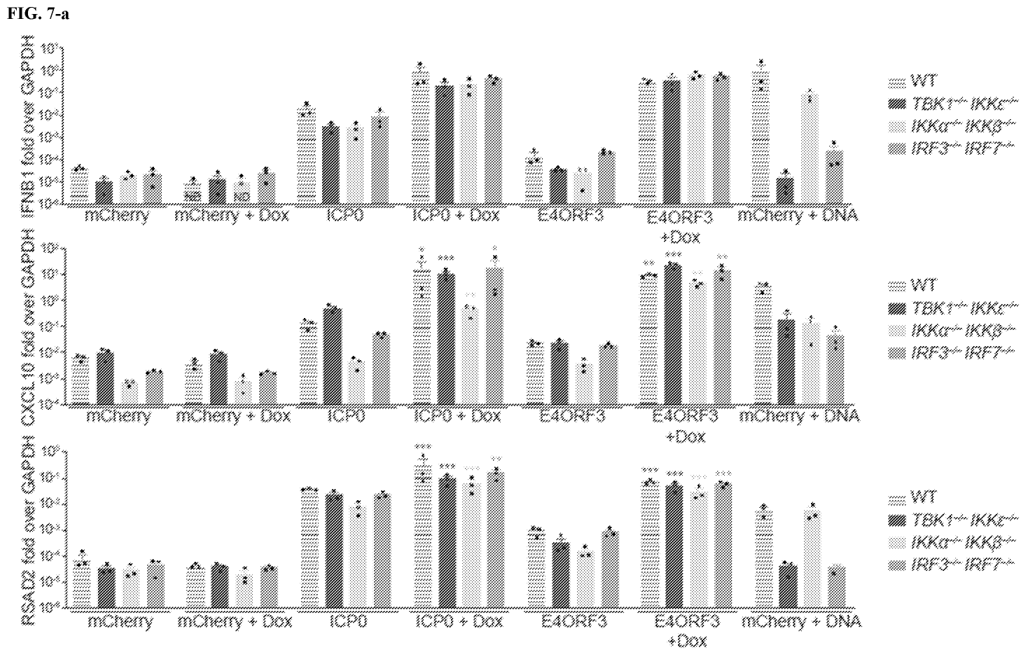

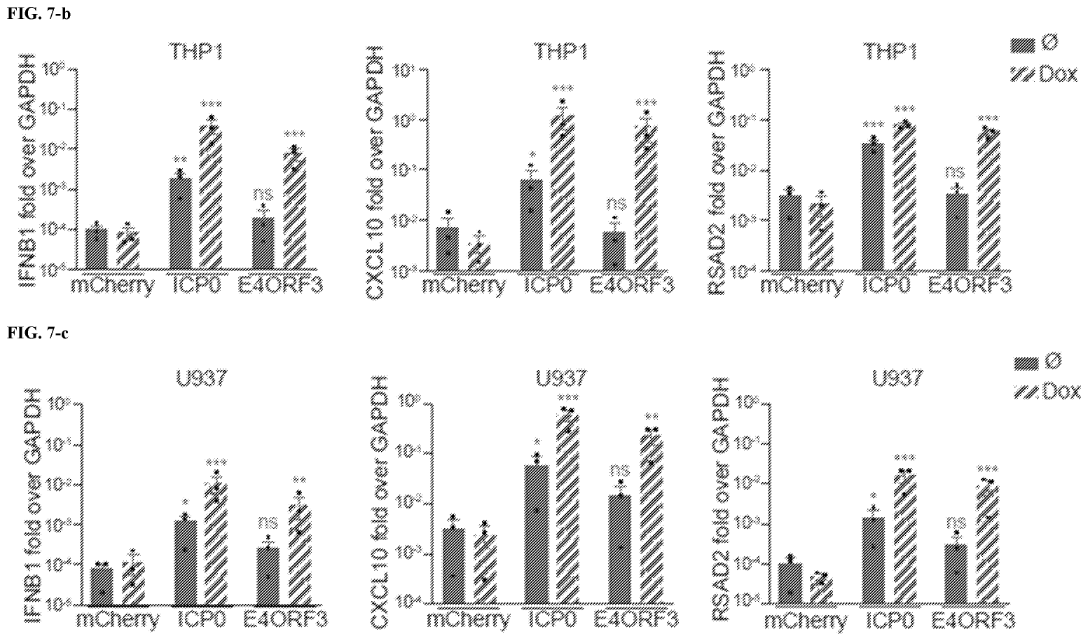

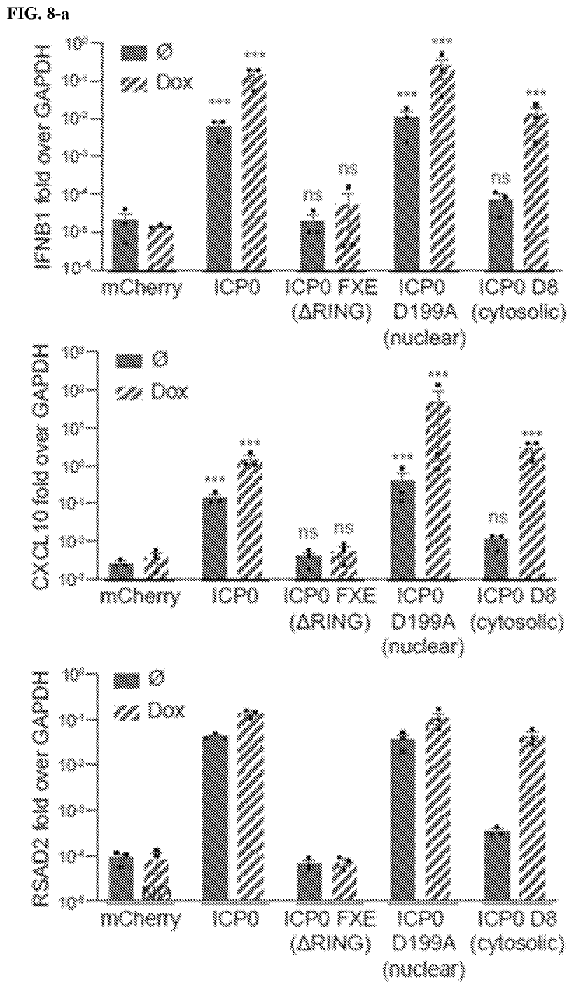

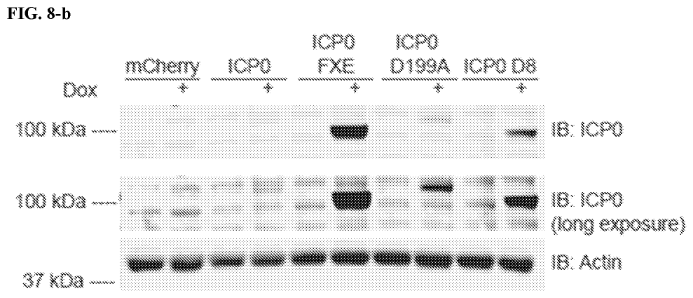

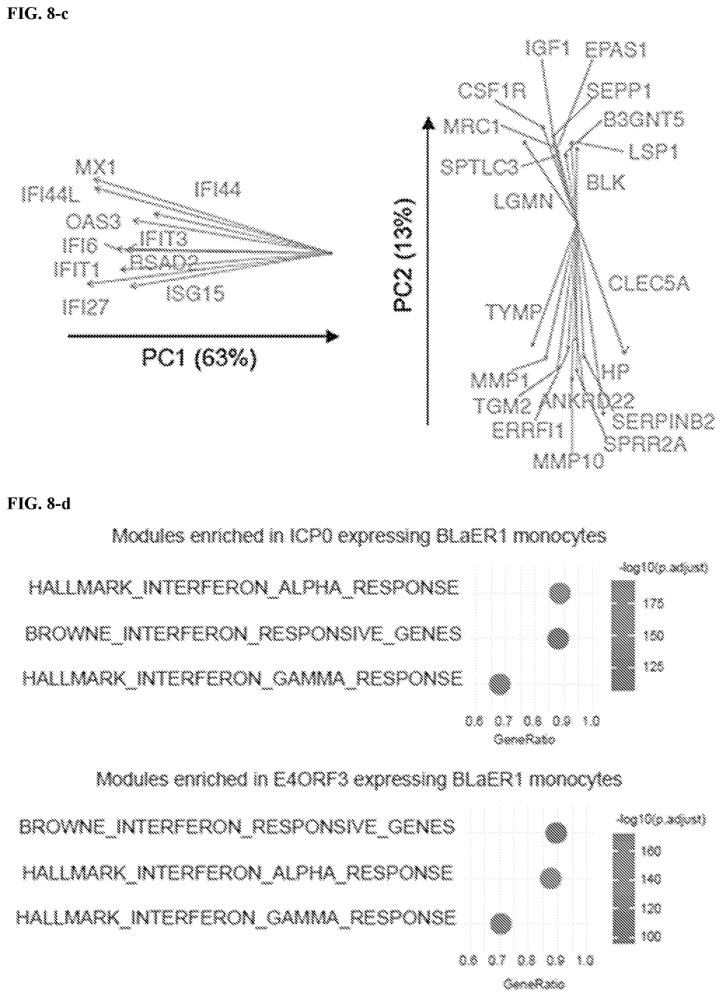

Next, ICP0 was ectopically expressed in human monocytes utilizing a doxycycline-inducible lentiviral system. Mirroring HSV-1 infection, ICP0 expression led to an IFN response that was independent of the PRR-signaling hubs TBK1/IKKε, IRF3/7 and IKKα/β (FIG. 1-d, FIG. 7-a). Thus, ICP0 is not only important but is also sufficient for IFN induction. The ability of the immune system to detect viral virulence factors was not limited to ICP0 from HSV-1. Ectopic expression of E4ORF3, a functionally equivalent virulence factor from Adenovirus 5, induced a potent IFN response independently of PRR signaling molecules (FIG. 1-d, FIG. 7-a). These observations were confirmed in THP1 and U937 cells, which also sensed both virulence factors to induce IFN (FIG. 7-b, FIG. 7-c). Leaky doxycycline-independent expression of ICP0 was sufficient to induce IFN, whereas E4ORF3 required doxycycline induction to be recognized (FIG. 7-a). The enzymatic function of IPC0 is likely to be active at low concentrations, whereas E4ORF3 polymerizes in order to sequester ND nuclear body components, which presumably requires higher expression levels. Consistent with its potent activity, ICP0 was hardly detected by immunoblot despite triggering a potent immune response (FIG. 8-b). An enzymatically inactive variant of ICP0 lacking the catalytic RING domain failed to induce IFN. The predominantly nuclear variant ICP0 D199A was not impaired in IFN induction whereas the predominantly cytosolic ICP0 D8 variant was less potent, especially at low doxycycline-independent expression levels (FIG. 8-a, FIG. 8-b). Thus, nuclear localization and catalytic activity of ICP0 were required for optimal innate immune activation.

To evaluate the global transcriptional consequences of sensing ICP0 and E4ORF3 by the innate immune system, RNAseq was performed. Principal component analysis identified interferon-stimulated genes (ISGs) comprising PC1 (FIG. 8-c) as the major discriminator between ICP0- and E4ORF3-expressing conditions and control cells (FIG. 1-e). Indeed, an enrichment analysis of genes with significantly increased transcription after virulence factor expression identified IFN signatures (FIG. 8-d) and multiple ISGs were activated strongly (FIG. 1-f). Co-clustering of virulence factor expressing conditions on the PCA plot indicated that the transcriptional response induced by ICP0 and E4ORF3 were overall comparable.

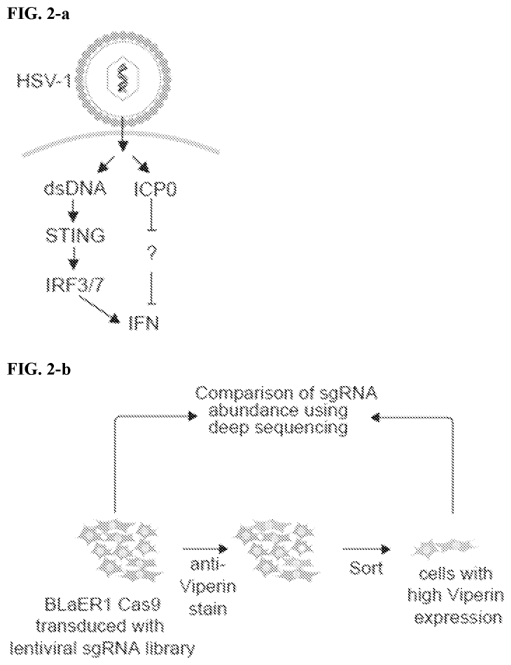

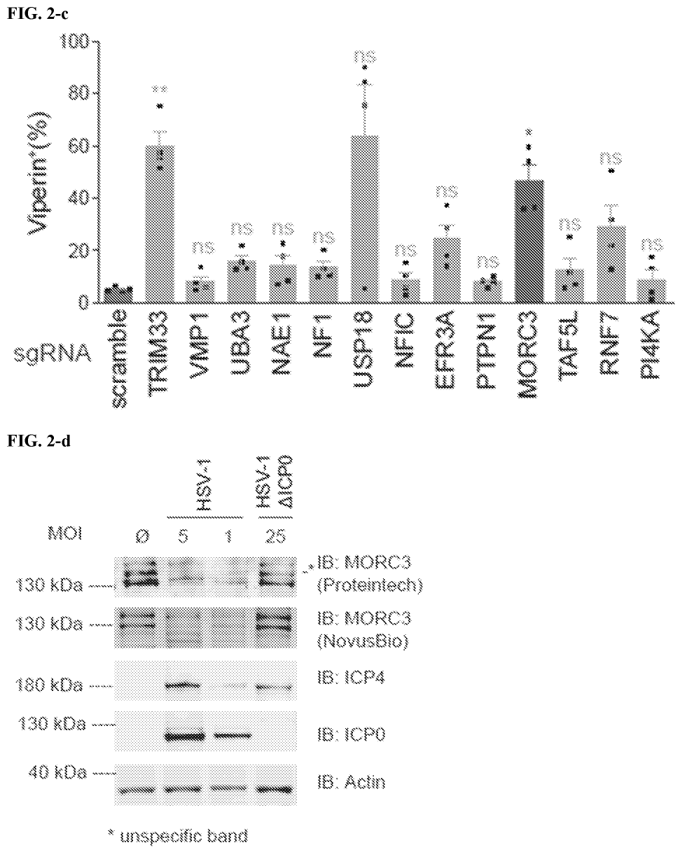

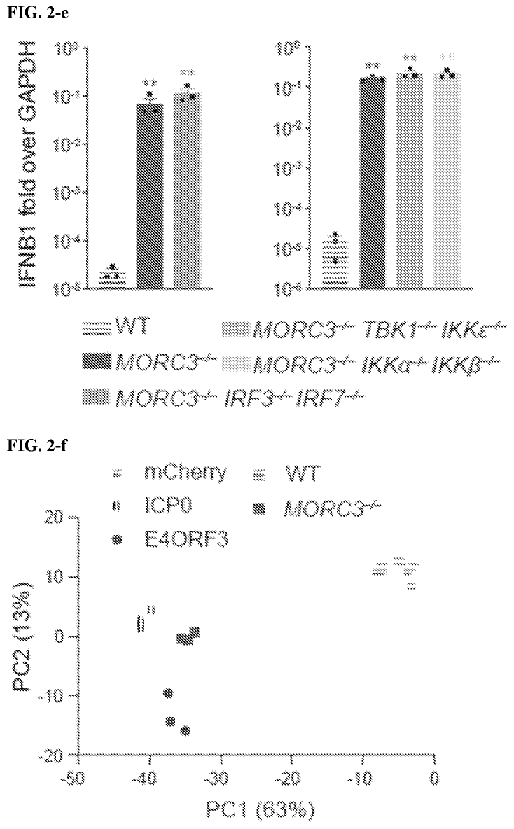

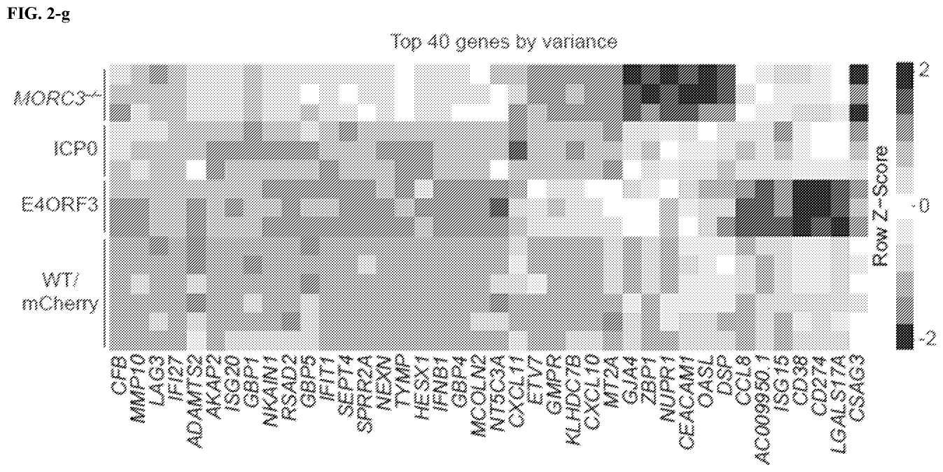

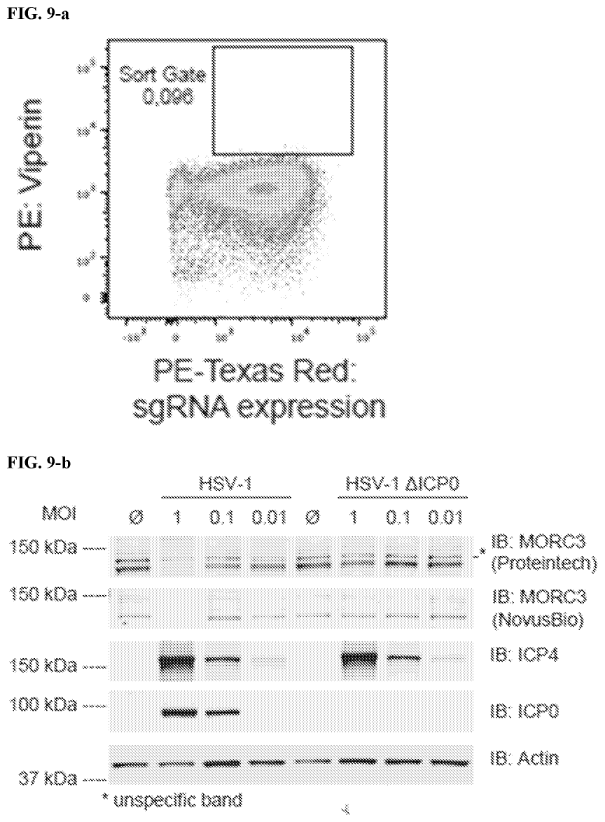

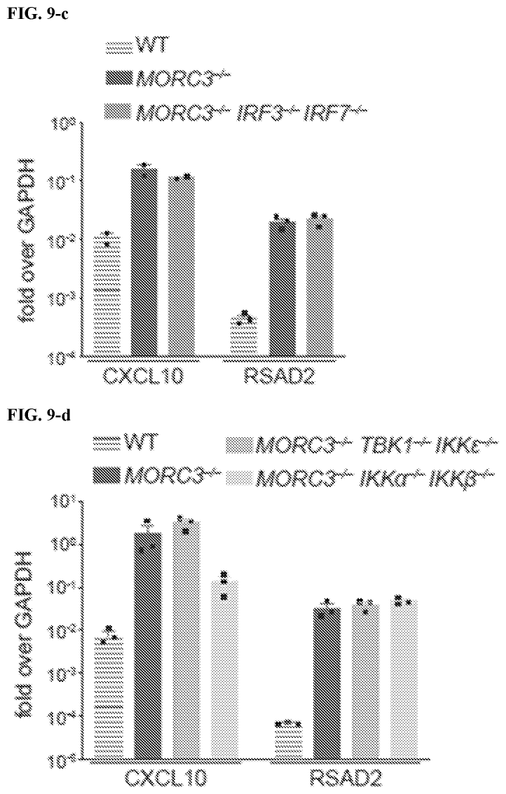

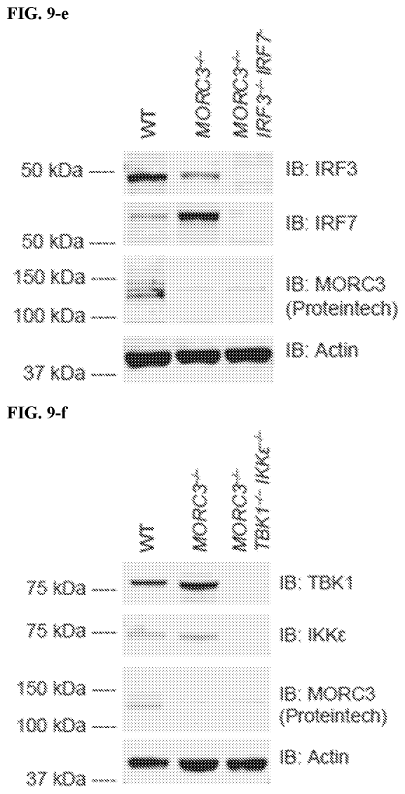

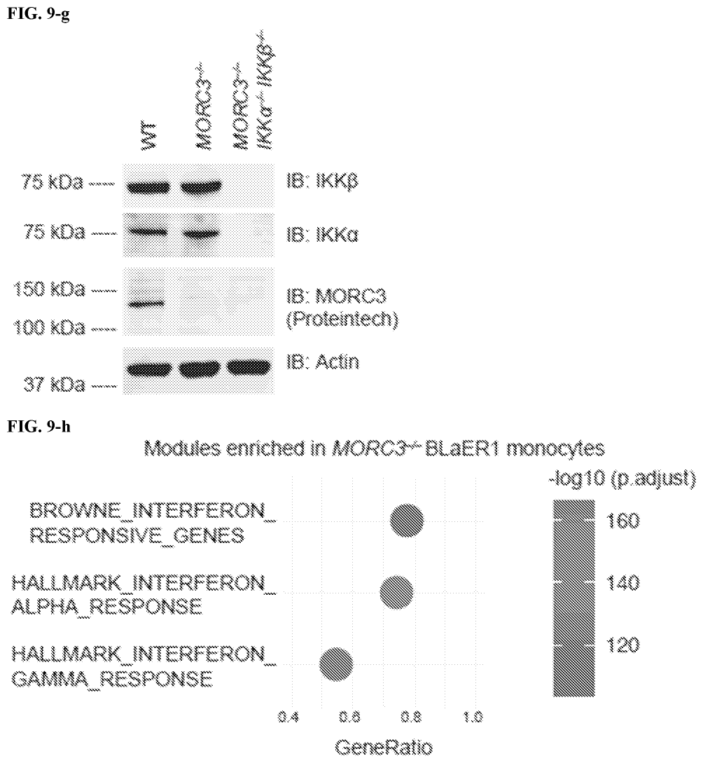

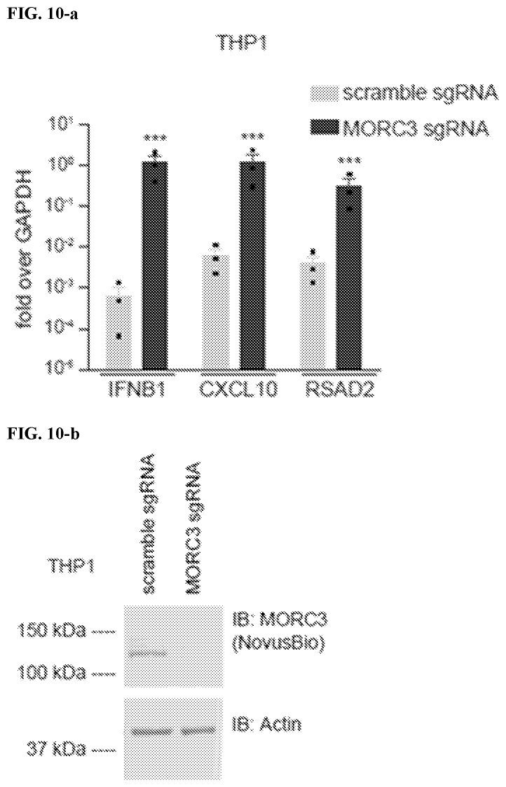

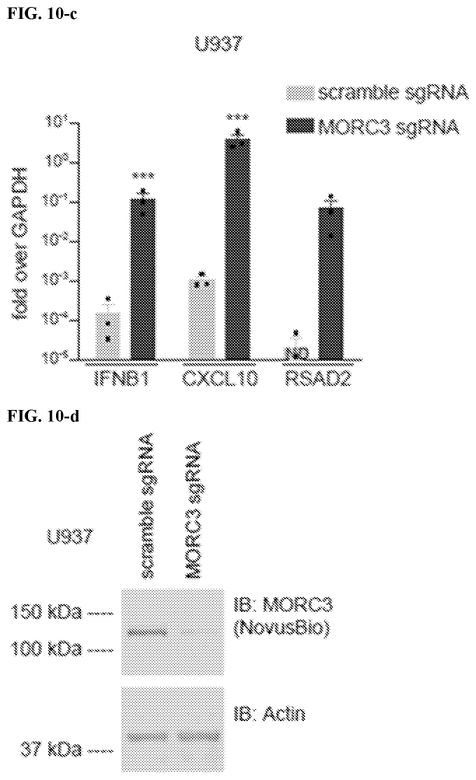

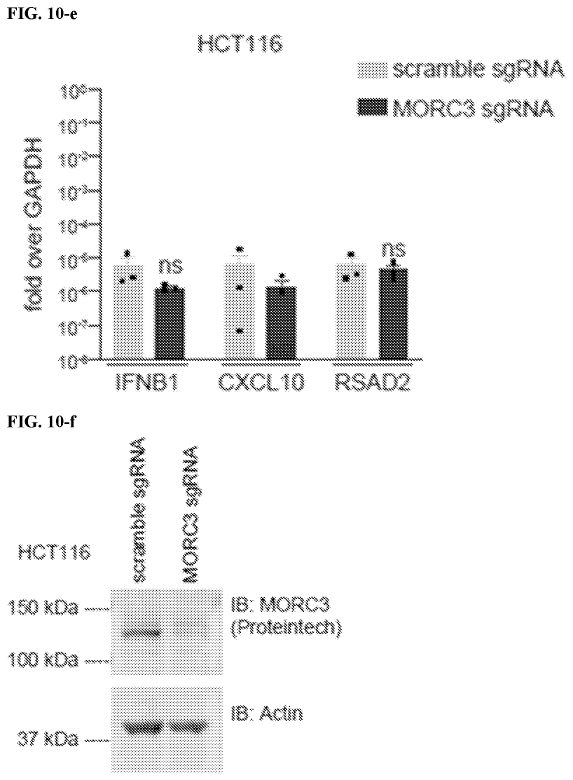

ICP0 displays SUMO-targeted ubiquitin ligase-like activity that marks sumoylated substrates for proteasomal degradation. Thus, its primary function is to degrade or inactivate host proteins. The hypothesis that ICP0 targets a negative regulator of IFN for degradation (FIG. 2-a) inadvertently triggering an IFN response was considered. This hypothesis is consistent with the prior observation that genetic perturbance of the sumoylation machinery activates IFN in human monocytic cell lines. To identify negative regulators of IFN in BLaER1 monocytes, a genome-wide CRISPR screen was conducted with the hope of identifying a nuclear, sumoylated substrate of ICP0 that negatively regulates IFN. The screen relied on antibody staining of the cytosolic ISG-product Viperin to report IFN-status via autocrine signaling at single cell resolution (FIG. 2-b). Cells with spontaneous induction of Viperin were sorted from the bulk population (FIG. 9-a) to enrich for sgRNAs whose targets are negative regulators of IFN under steady state conditions. Several individual sgRNAs were validated to enhance Viperin expression at steady state with varying effect sizes (FIG. 2-c), including sgRNAs targeting TRIM33, a known negative regulator of PRR-mediated IFN induction, and USP18, a known negative regulator of IFNAR signaling. In addition, sgRNAs targeting the sumoylated nuclear body protein MORC3 induced Viperin expression. MORC3 was focused on as it was previously described to be degraded by ICP0 in fibroblasts. It was confirmed that ICP0 was required for MORC3 degradation during HSV-1 infection in BLaER1 monocytes (FIG. 2-d) and U2OS cells, which support ICP0-independent HSV-1 replication (FIG. 9-b). Independently generated MORC3^{-/-} BLaER1 monocytes (FIG. 2-e, FIG. 9-c-FIG. 9-g), or MORC3-targeted THP1 (FIG. 10-a, FIG. 10-b) and U937 (FIG. 10-c, FIG. 10-d) myeloid-like cells displayed spontaneous IFN induction. Mirroring the characteristics of ICP0-mediated IFN, the IFN response in MORC3^{-/-} BLaER1 cells did not require additional PRR activation and was independent of known PRR-signaling hubs TBK1/IKKε, IRF3/7 and IKKα/β (FIG. 2-e, FIG. 9-c-FIG. 9-g). Similarly, global transcriptomic changes in MORC3^{-/-} monocytes recapitulated the transcriptional changes induced by virulence factors. This led to co-clustering of these conditions on the PCA plot (FIG. 2-f), co-upregulation of ISGs by heatmap analysis (FIG. 2-g), and enrichment of the same IFN-related modules in MORC3^{-/-} monocytes (FIG. 9-h). These results identify MORC3 as a negative regulator of IFN in human monocytes and demonstrate that genetic loss or ICP0-mediated degradation of MORC3 leads to a potent IFN response that is independent of PRR-signaling hubs.

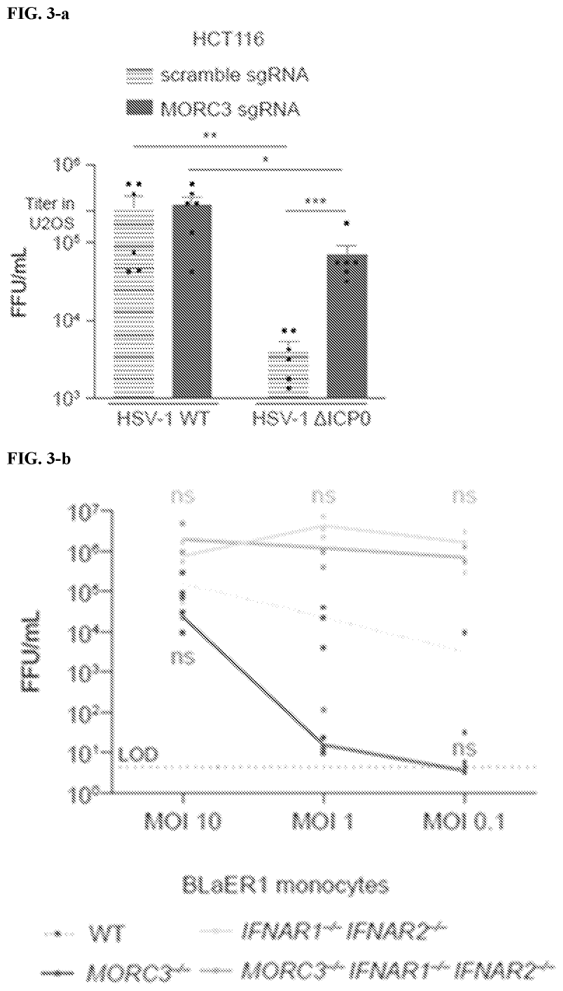

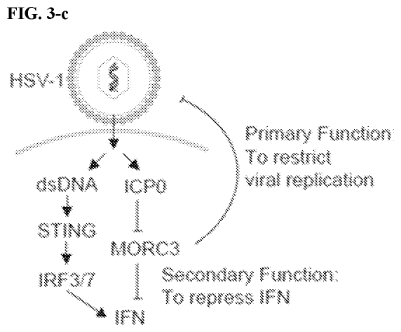

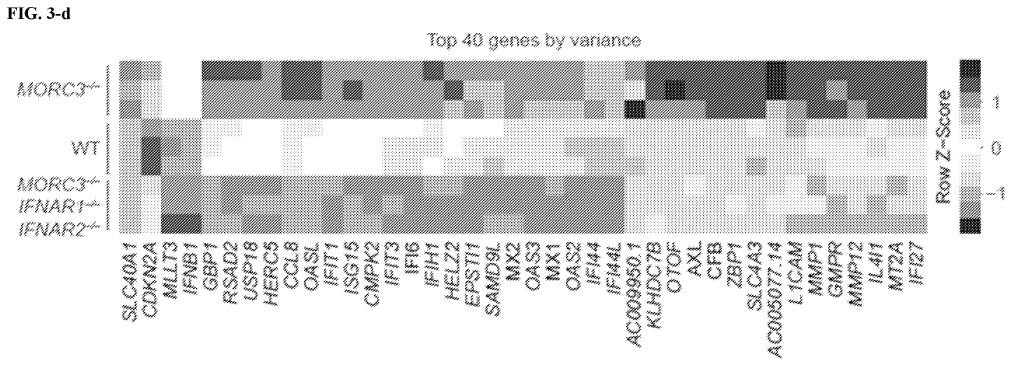

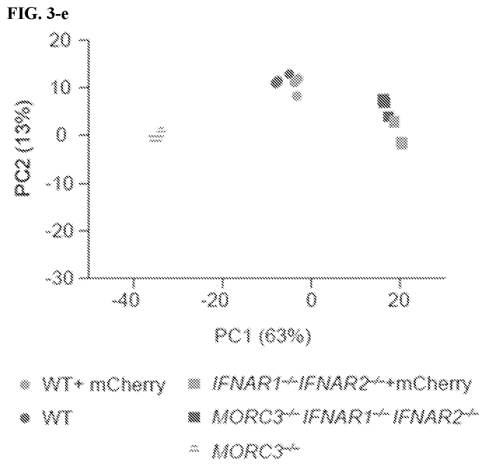

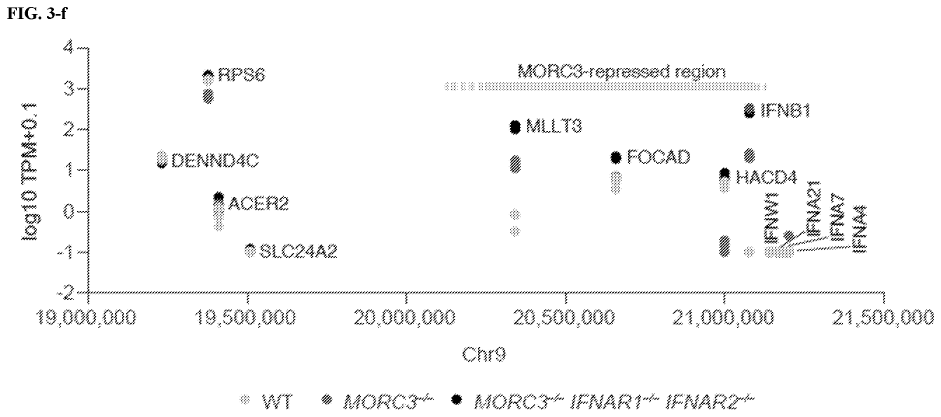

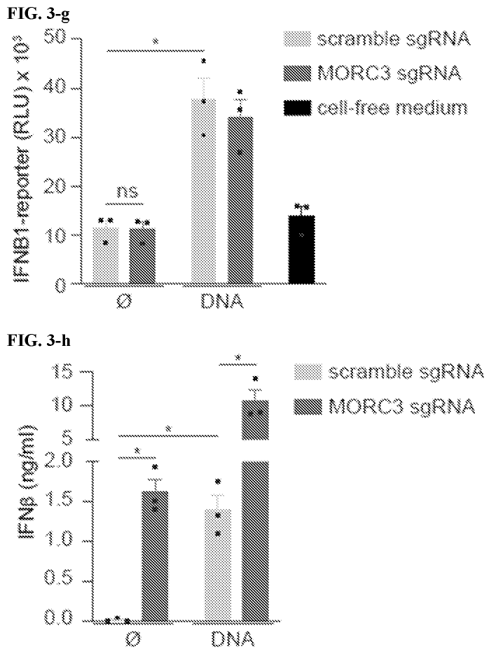

MORC3 is a member of the MORC-gene family of GHKL (gyrase, Hsp90, histidine kinase, MutL)-type ATPases that are transcriptional repressors. MORC3 has been proposed to bind H3K4me3 at promoters and participate in SUMO2-mediated transcriptional repression. It co-localizes with ND10 nuclear bodies and is required for p53 activation and senescence induction in response to genotoxic stress. In mice, MORC3 is embryonically lethal and partial loss of MORC3 impairs senescence in osteoclasts and induces their longevity, driving osteoporosis and related immune pathology. Localization of MORC3 to anti-viral ND10 nuclear bodies suggests a role for MORC3 during viral infection. Whereas one report observed enhanced viral replication in MORC3 knockdown cells (implying an anti-viral function of MORC3), other studies reported diminished viral replication in MORC3 knockdown cells and increased viral replication upon MORC3 overexpression (implying a pro-viral function of MORC3). The data herein suggest that MORC3 has two functions: (1) a primary function to directly repress viral replication; and (2) a secondary function to repress IFN/ISG induction. The primary function of MORC3 is apparent in cells that do not induce IFN upon MORC3 loss, such as HCT116 cells (FIG. 10-e, FIG. 10-f). In these cells, loss of MORC3 results in enhanced replication of ΔICP0 HSV-1 (FIG. 3-a), confirming that HSV-1 is subject to MORC3-mediated restriction. WT HSV-1 employs ICP0 to degrade MORC3 to alleviate this restriction. Consequently, only ΔICP0 HSV-1 benefited from the deletion of MORC3 (FIG. 3-a). In monocytic cells, in which loss of MORC3 results in IFN induction, MORC3^{-/-} cells restrict viral replication. This is due to elevated IFN in the absence of MORC3 because viral replication was fully restored in MORC3^{-/-} IFNAR1^{-/-} IFNAR2^{-/-} cells (FIG. 3-b). Therefore, the primary anti-viral function of MORC3 may be guarded by its secondary IFN-repressing function. These two functions are intimately linked such that viruses that express virulence factors to degrade MORC3, and avoid its primary anti-viral function, will unleash the secondary anti-viral interferon response (FIG. 3-c). Combining both guarded and guarding function in one protein generates the molecular equivalent of a dead-man's switch—a self-monitoring, self-insured anti-viral immune pathway.

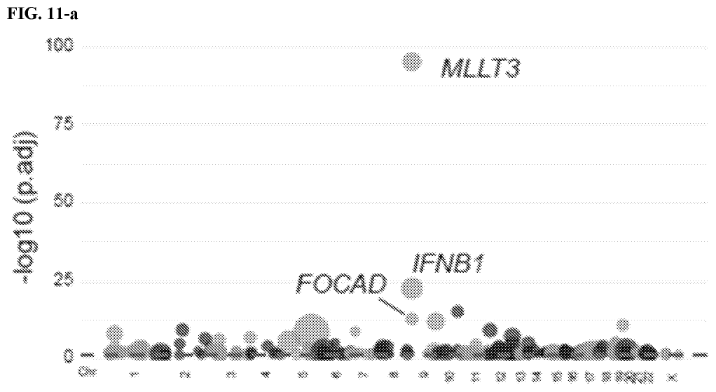

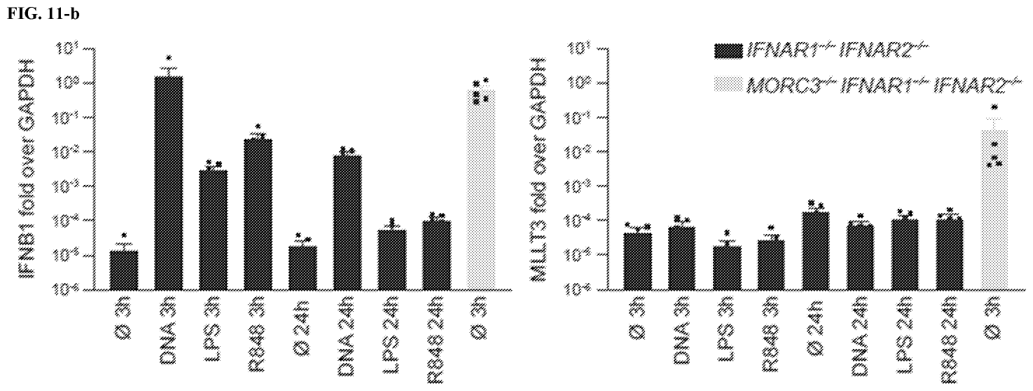

To investigate how MORC3 represses IFN/ISGs RNAseq of MORC3^{-/-} versus MORC3^{-/-}IFNAR1^{-/-}IFNAR2^{-/-} monocytes was performed. It was found that the majority of transcriptional changes incurred by MORC3 deficiency were due to IFNAR signaling. ISGs that were induced in MORC3^{-/-} cells were not upregulated upon IFNAR co-deletion (FIG. 3-d) and MORC3^{-/-}IFNAR1^{-/-}IFNAR2^{-/-} cells clustered together with IFNAR1^{-/-}IFNAR2^{-/-} cells on the PCA plot (FIG. 3-e). This indicates that MORC3 is not required to directly repress antiviral ISGs. However, a small number of genes, including IFNB1, were still de-repressed in MORC3^{-/-}IFNAR1^{-/-}IFNAR2^{-/-} monocytes, which suggests they may be direct targets of MORC3 repression (FIG. 3-d). The most significantly de-repressed genes in MORC3^{-/-}IFNAR1^{-/-}IFNAR2^{-/-} monocytes are MLLT3, IFNB1 and FOCAD, which, remarkably, are clustered together within a short section of chromosome 9 (chr9:20,329,000-21,086,000; FIG. 11-a). Almost all protein-coding genes within this region were de-repressed in MORC3^{-/-} cells, while adjacent genes in both directions were not MORC3-regulated (FIG. 3-f). Transcriptional up-regulation of these genes was not a general feature of IFNB1 activation because PRR-induced transcription of IFNB1 did not induce MLLT3 transcription despite inducing IFNB1 to similar levels compared to MORC3^{-/-} cells (FIG. 11-b). Given that these genes are not normally co-regulated or functionally related, MORC3 may act in a locus-specific manner to regulate gene expression. Consistent with this, a retroviral-based randomly integrated IFNB1-promoter-luciferase reporter was only triggered by DNA-STING signaling but was not activated in the absence of MORC3 (FIG. 3-g). As expected, the endogenous IFNB1 gene was activated both by STING signaling and MORC3 deficiency (FIG. 3-h).

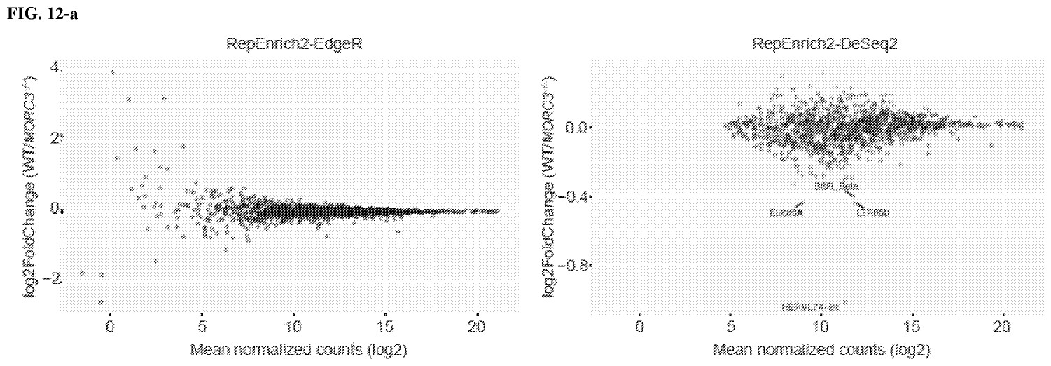

In murine embryonic stem cells, MORC3 participates in repression of endogenous retroviruses (ERVs). However, only a minimal de-repression of ERVs in MORC3 deficient monocytes (FIG. 12-a) was found. The only strongly de-repressed ERV family upon MORC3 deficiency includes members within the MORC3-repressed region on chromosome 9, consistent with a positional rather than ERV-specific de-repression. ERV repression likely occurs by distinct mechanisms in embryonic stem cells and monocytes, with MORC3-independent ERV repression mechanisms, such as DNA methylation, predominating in monocytes. Overall, these results indicate that MORC3 acts to repress the IFNB1 locus rather than the IFNB1 promoter, whereas canonical PRR-mediated induction of IFNB1 is promoter-dependent and locus-independent.

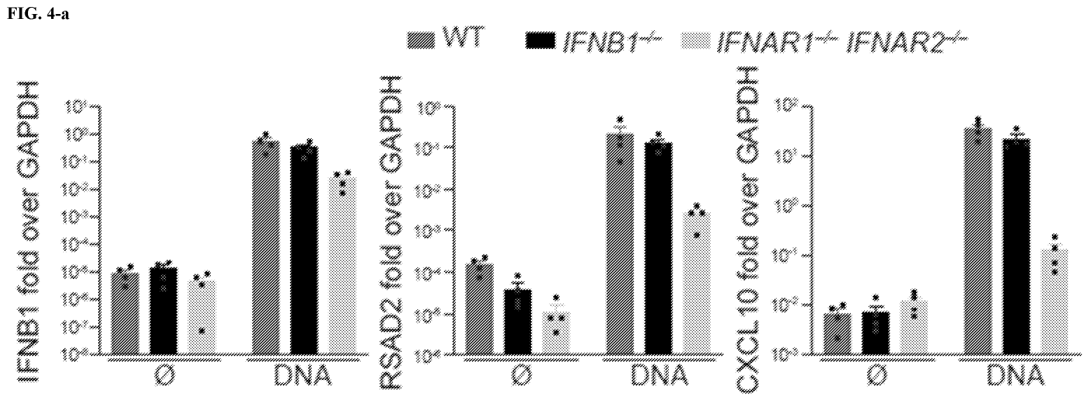

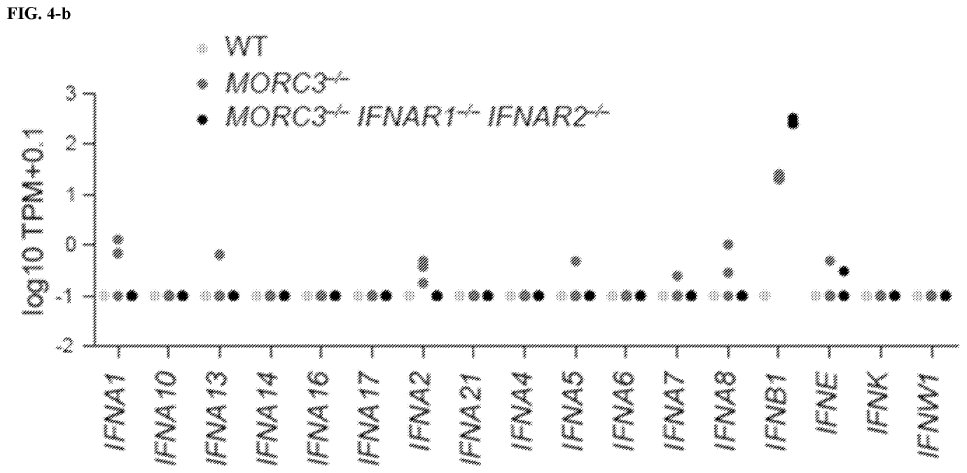

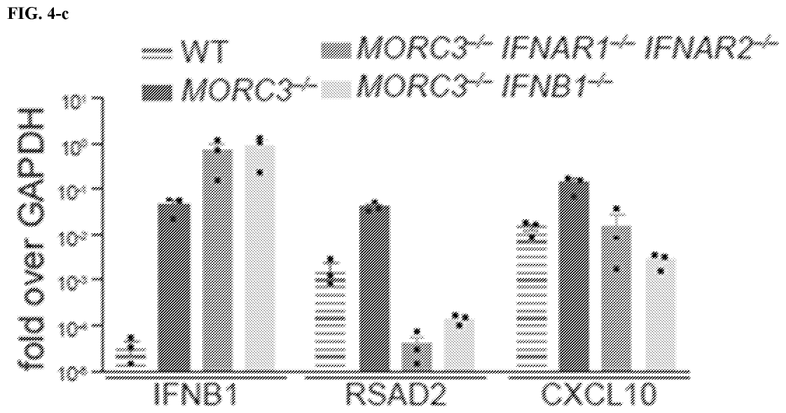

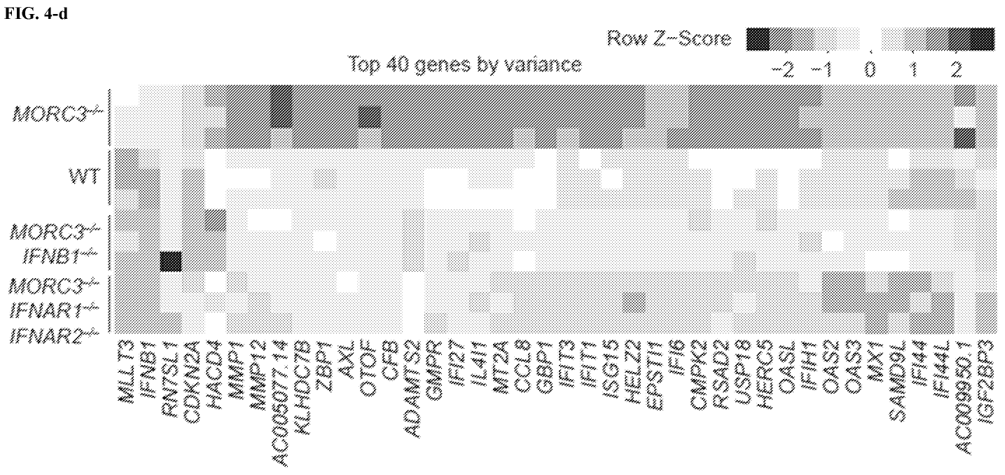

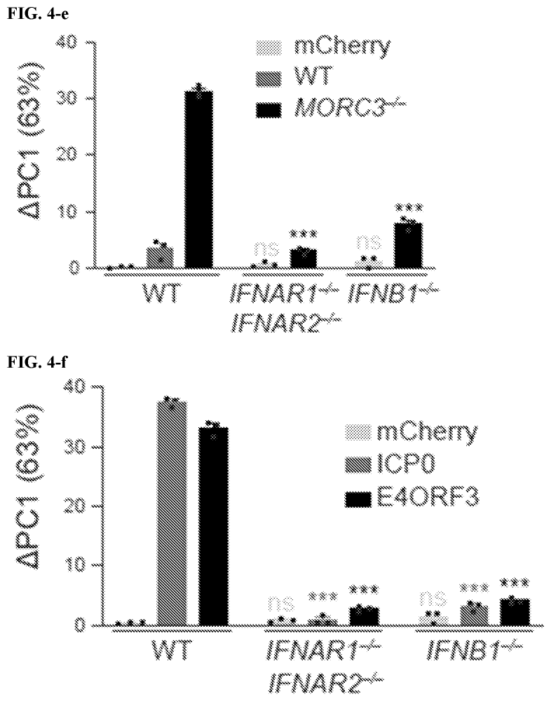

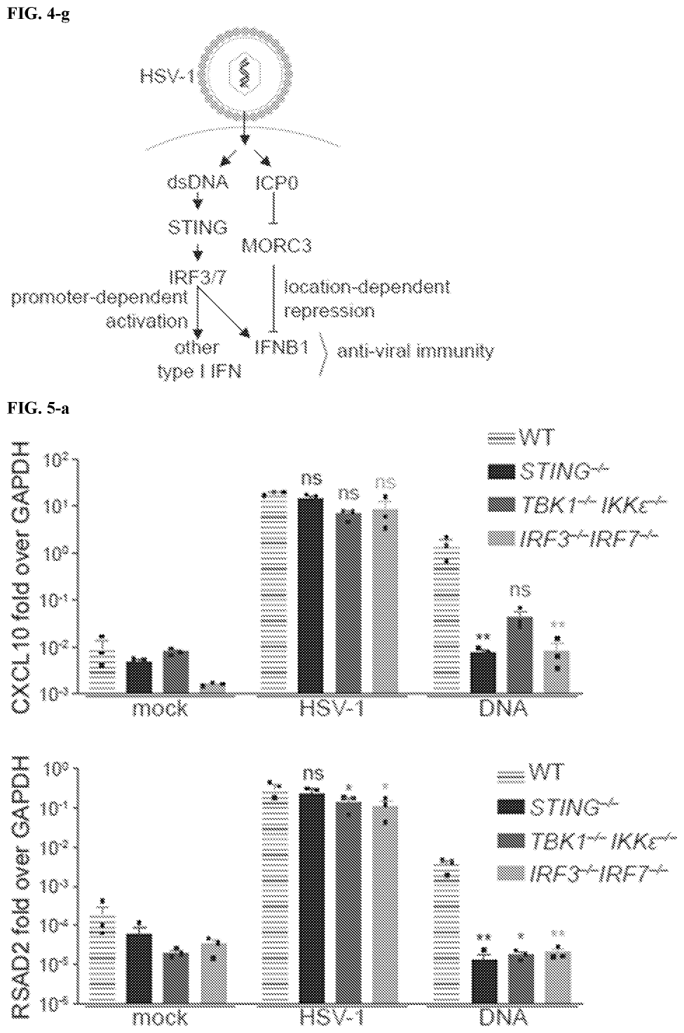

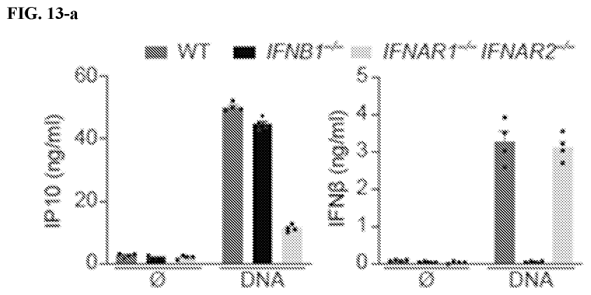

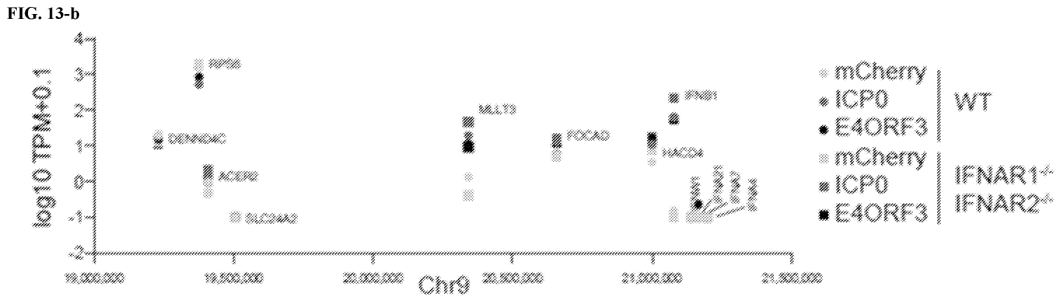

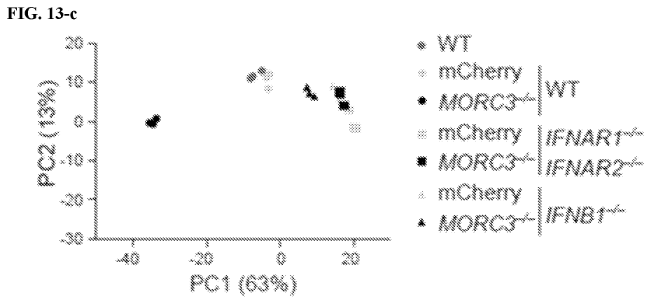

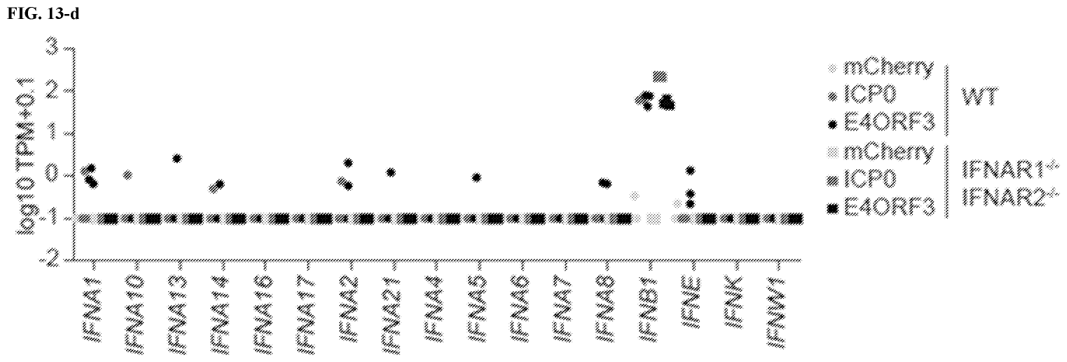

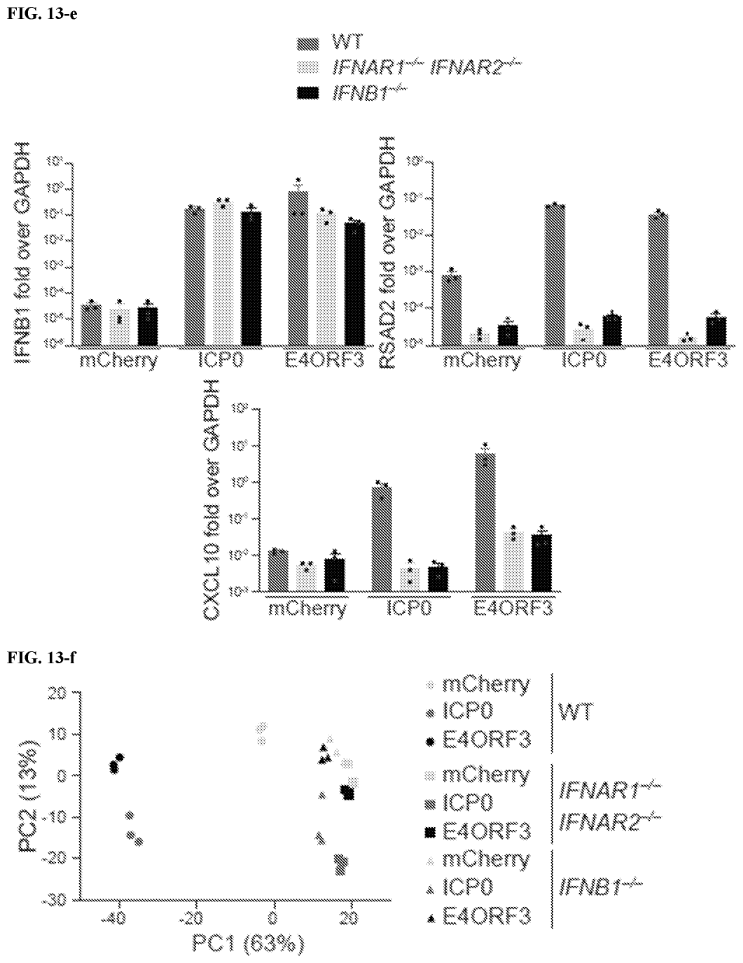

Finally, how the locus-specific repression mediated by MORC3 regulates expression of anti-viral ISGs was investigated. There are 17 different type I IFN genes (IFNB1, IFNE, IFNK, IFNW1, and thirteen IFNA genes) that cluster together on chromosome 9 and encode proteins that vary in cell-type expression, kinetics of induction, and receptor affinity. The most studied type I IFN gene is IFNB1 because it is dominantly induced in IFN-producing cells. However, ISG induction downstream of STING activation in BLaER1 monocytes did not rely solely on IFNB1, as revealed by IFNAR-dependent induction of ISGs, such as RSAD2, CXCL10, in an IFNB1-independent manner in response to foreign DNA (FIG. 4-a, FIG. 13-a). Thus, BLaER1 monocytes are competent to make IFNs other than IFNB1. In contrast, IFNB1 is the only IFN-gene within the MORC3-repressed genomic region on chromosome 9 (FIG. 3-d) and is thus the only IFN-gene that is de-repressed in MORC3^{-/-} cells (FIG. 4-b). Consequently, monocytes required IFNB1 to activate ISGs in the absence of MORC3 (FIG. 4-c, FIG. 4-d) but not after STING activation (FIG. 4-a). MORC3^{-/-}IFNB1^{-/-} cells clustered together with MORC3^{-/-}IFNAR1^{-/-}IFNAR2^{-/-} on the PCA plot of RNAseq data (FIG. 13-c) and MORC3 deletion did not induce separation along the PC1 axis of ISG expression in IFNB1^{-/-} cells (FIG. 4-e). As expected, virulence factor-mediated activation of the MORC3 pathway also resulted in the locus-specific induction of IFNB1 and its neighboring genes (FIG. 13-b), only de-repressed IFNB1 of all IFN-genes (FIG. 13-d), and required IFNB1 for induction of ISGs (FIG. 4-f, FIG. 13-e, FIG. 13-f). Thus, MORC3 acts narrowly and selectively on the IFNB1 locus and does not regulate other linked type I IFN genes. Thus, in contrast to canonical PRR-signaling, activation of the MORC3 pathway by viral virulence factors uniquely relies on IFNB1 for anti-viral ISG induction (FIG. 4-g).

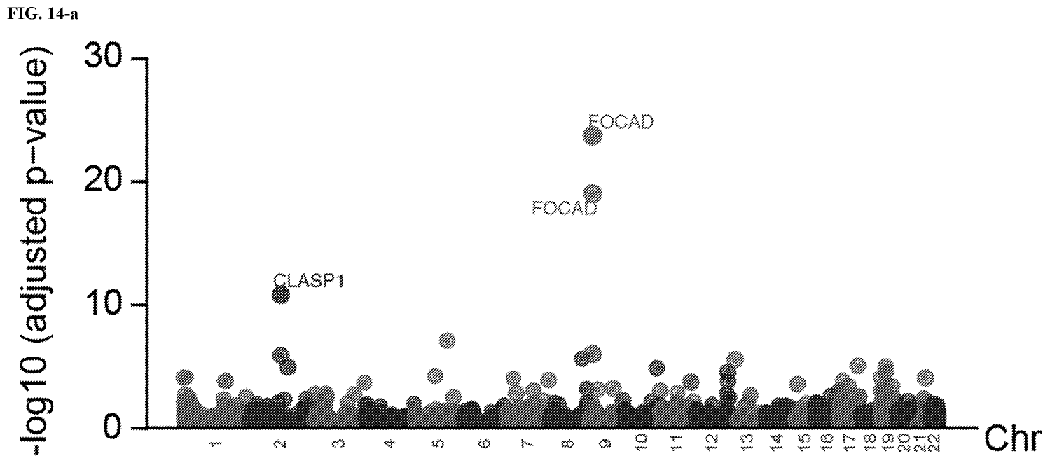

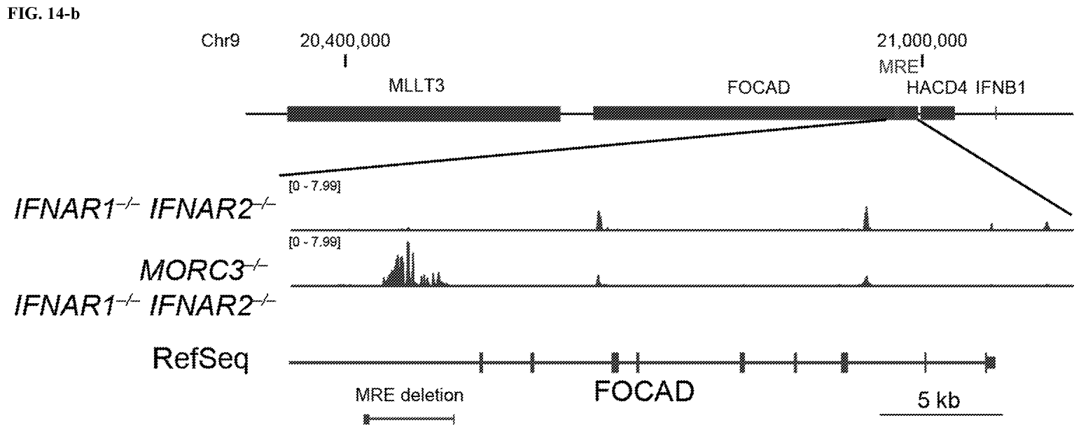

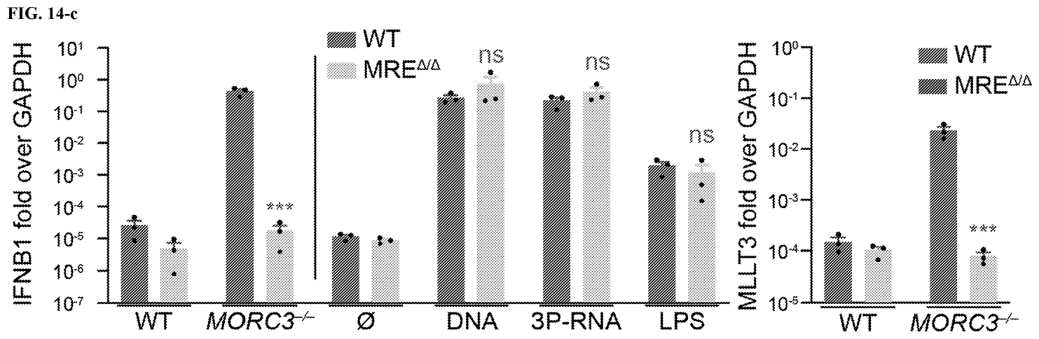

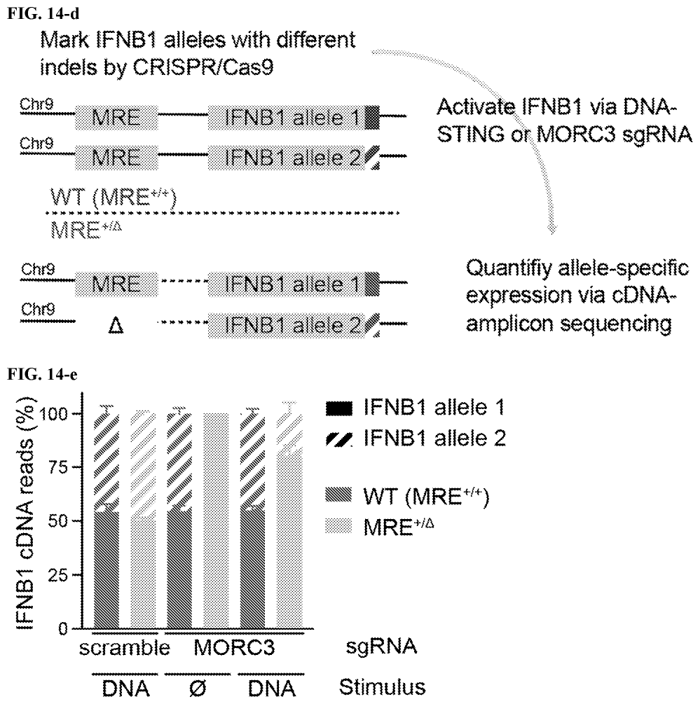

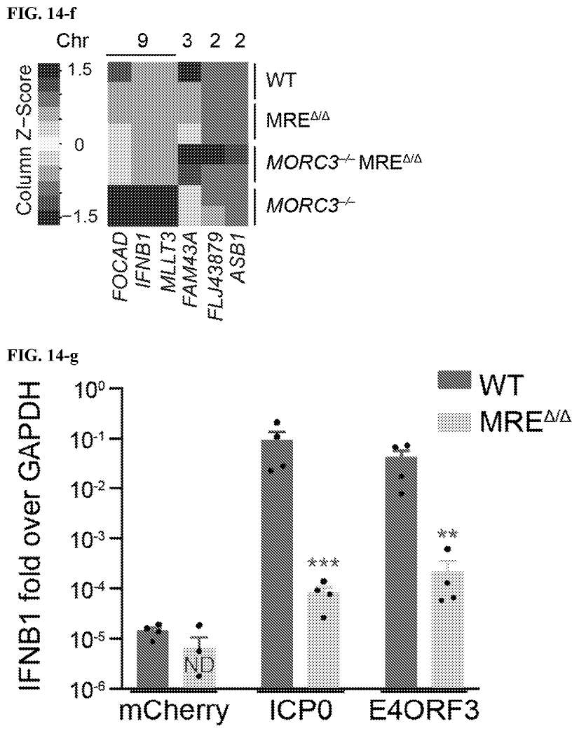

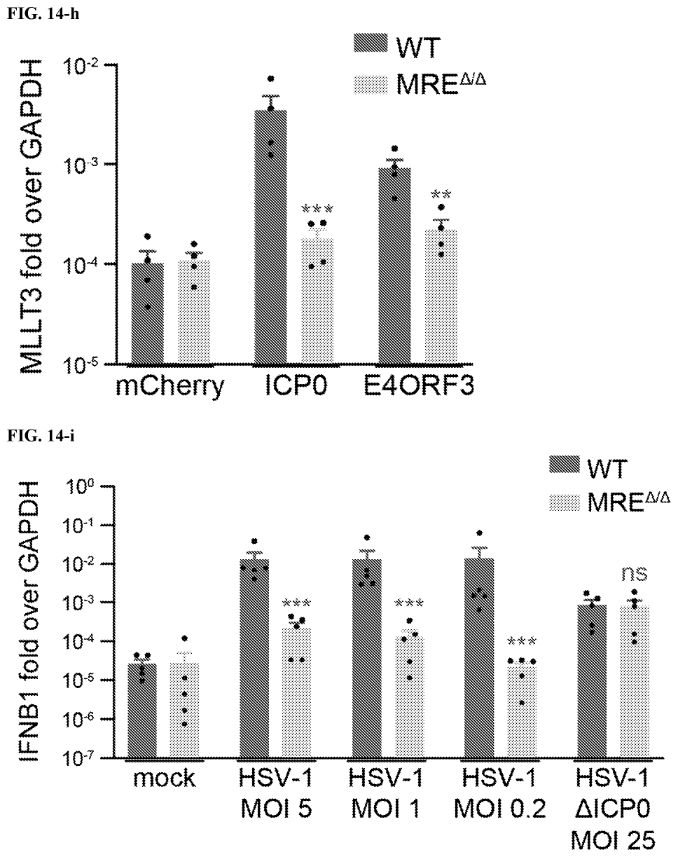

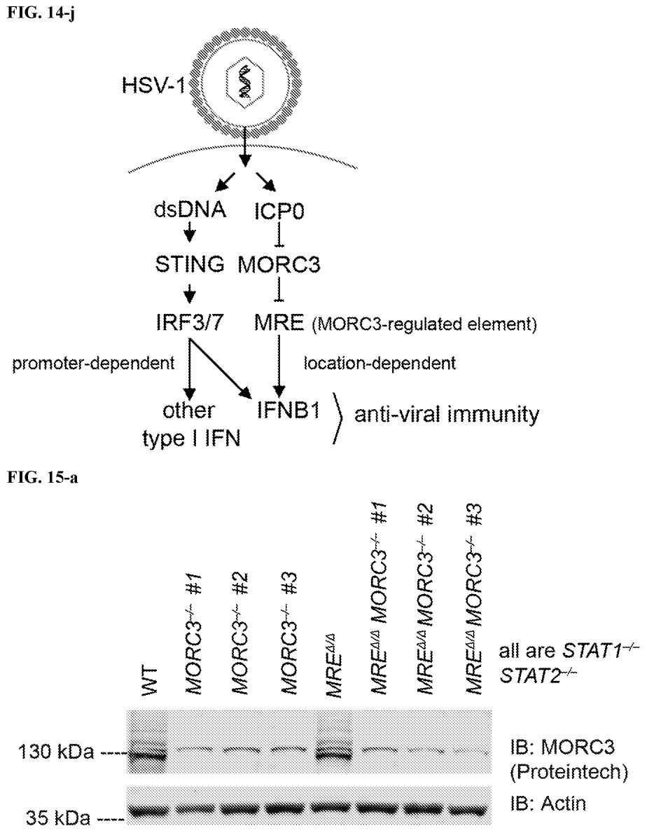

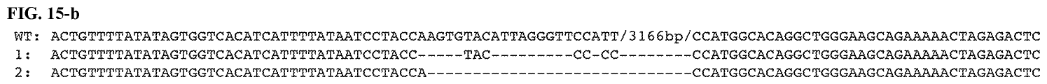

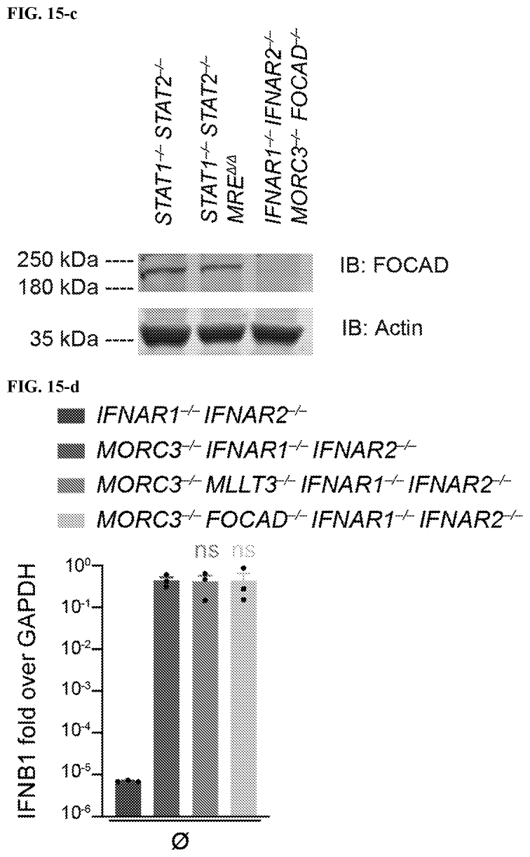

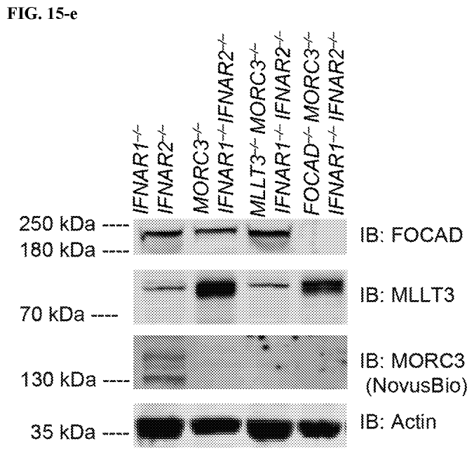

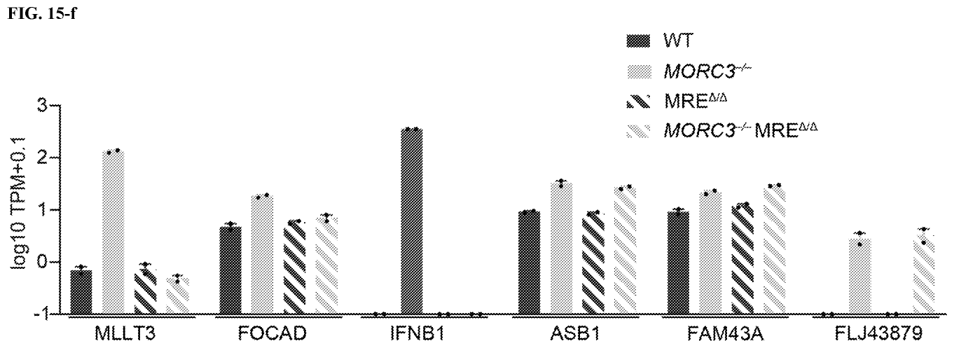

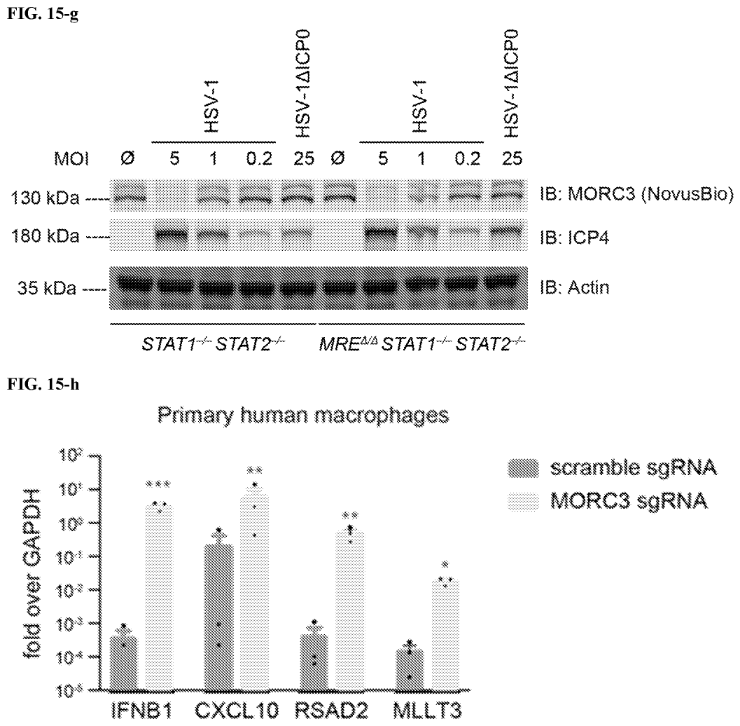

ATAC-seq was used to identify a minisatellite-like element, which is referred to herein as the MORC3-regulated element (MRE), in an intron of FOCAD, approximately 100 kb downstream of IFNB1, that gains DNA accessibility in the absence of MORC3 (FIG. 14-a, FIG. 14-b). The MRE sequence is set forth as SEQ ID NO: 3. The MRE was required for IFNB1 and MLLT3 de-repression in MORC3^{-/-} cells but not for IFNB1 induction upon PRR signaling, indicating that the MRE is a specific component of the MORC3 pathway (FIG. 14-c, FIG. 15-a, FIG. 15-b). Although the MRE is located in an intron of FOCAD, FOCAD protein expression was not impaired in MRE^{Δ/Δ} cells, and neither FOCAD nor MLLT3 were required for IFNB1 induction in absence of MORC3 (FIG. 15-c-FIG. 15-e). It was found that MRE regulates IFNB1 in cis because a MORC3-sgRNA drove bi-allelic expression in WT cells and mono-allelic expression of IFNB1 in heterozygous MRE^{+/Δ} cells (FIG. 14-d, FIG. 14-e). Confirming the regulation in cis, only chromosome-9-located high-confidence MORC3-repressed genes (identified in all RNA-seq conditions) required the MRE to be induced in absence of MORC3 FIG. 14-f). The effect size of de-repression of genes outside of the chromosome 9 locus is minor (FIG. 15-f), indicating that the regulation of IFNB1 and surrounding genes is the main effect of the MORC3 pathway in monocytes. Consistent with the importance of the MRE as a component of the MORC3 pathway, it was required for ectopically expressed virulence factors to induce IFNB1 and MLLT3 transcription (FIG. 14-g, FIG. 14-h) as well as ICP0-dependent IFNB1 induction during HSV-1 infection (FIG. 14-i). As ΔICP0 HSV-1 is attenuated, a 25× higher MOI was used, thereby leading to comparable ICP4 expression between WT and ΔICP0 HSV-1 (FIG. 15-g). MORC3 was still degraded by ICP0 in absence of the MRE, confirming that MORC3 degradation is upstream of MRE activation (FIG. 15-g). To confirm key aspects of the MORC3 pathway in primary immune cells, MORC3 was targeted in primary human macrophages and a strong, spontaneous upregulation of IFNB1, MLLT3, and ISGs (FIG. 15-h) was observed. In summary, MORC3 represses a regulatory DNA element (MRE). Upon viral degradation or genetic deletion of MORC3, the MRE mediates activation of adjacent genes in cis which explains the location-specific de-repression of IFNB1.

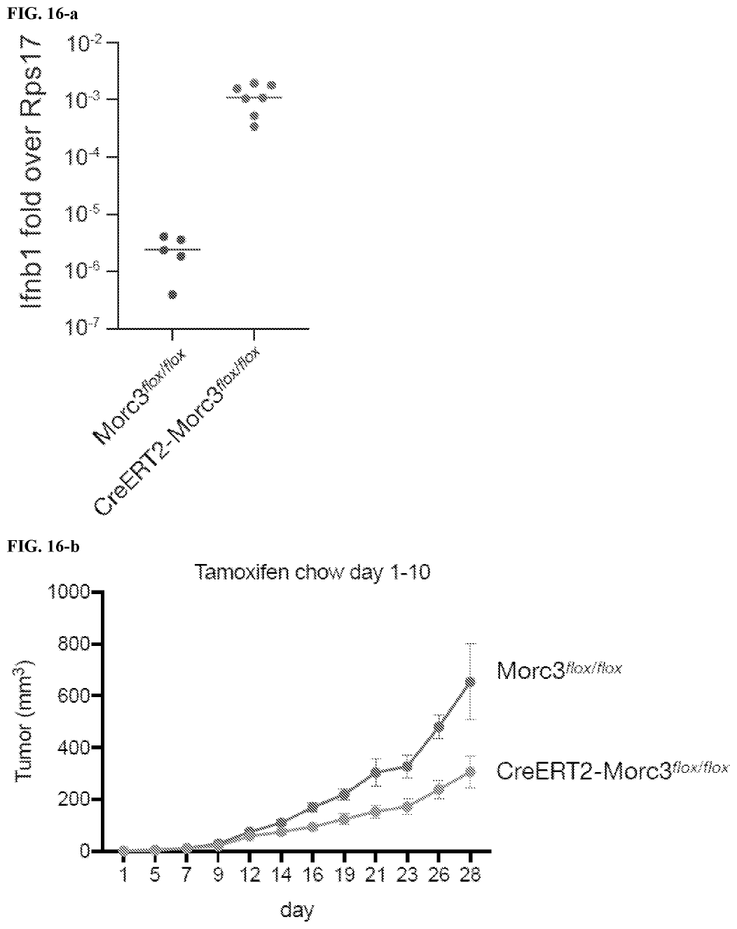

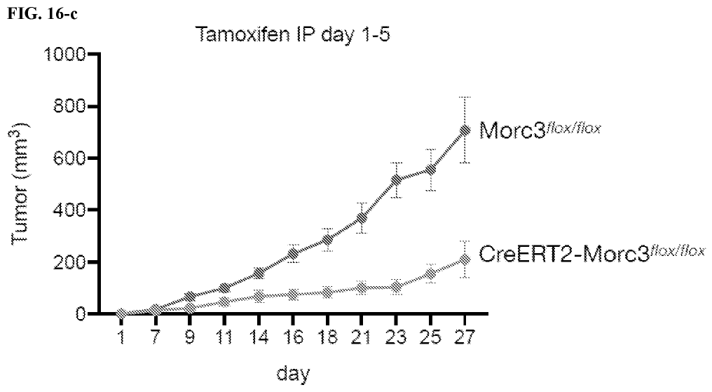

To assess whether elimination of MORC3 can result in anti-tumor activity, mice harboring a conditional (floxed) allele of Morc3 were generated. These mice were then crossed to mice harboring an inducible CreERT2 gene. In the presence of tamoxifen, CreERT2 activity is induced, resulting in excision (deletion) of the Morc3 gene. Loss of Morc3 results in ˜100-fold Ifnb induction in the spleens of mice (FIG. 16-a), consistent with the finding above that Morc3 is an essential negative regulator of interferon expression. The loss of Morc3 also resulted in enhanced tumor clearance (FIG. 16-b, FIG. 16-c). Tumor clearance was observed whether tamoxifen was provided orally (FIG. 16-a) or via intraperitoneal injection (FIG. 16-c).

These results identify the MORC3-pathway as a novel innate immune sensing mechanism that detects the enzymatic activity of virulence factors from DNA viruses. During HSV-1 infection of monocytes, the MORC3 pathway induces a strong IFNB1 response redundantly with the cGAS-STING-pathway of cytosolic DNA sensing. As the STING-pathway is solely required for IFN induction during HSV-1 infection in other cell types, the MORC3-pathway might rely on monocyte-specific components that remain to be identified. Importantly, the MORC3-pathway does not utilize a PAMP-sensing receptor akin to a PRR; nor does it depend on canonical PRR signaling components that many viruses have evolved to disrupt or inhibit. Instead, it employs the self-guarded protein MORC3, whose bi-functionality allows detection of pathogen-encoded enzymatic activities. The primary function of MORC3 appears to be to inhibit replication of HSV-1 and likely other (DNA) viruses that replicate in the nucleus, a function that is presumably connected to its association with ND10 nuclear bodies. To escape restriction by MORC3, DNA viruses employ virulence factors, e.g. HSV-1 ICP0 and Ad5 E4ORF3, to inhibit ND10 nuclear bodies and/or degrade MORC3. In response, MORC3 may have evolved a secondary function: locus-specific repression of the IFNB1 gene. This secondary function allows activation of anti-viral IFN upon virulence factor-mediated perturbation of ND10 nuclear bodies and MORC3. De-repression of the IFNB1 locus may be less susceptible to pathogen interference or tumor cell evasion as compared to a multi-step PRR signaling pathway to activate IFNB1 transcription. The MORC3 pathway might also explain prior observations of IRF3/7-independent IFNβ during DNA virus infections and connections between perturbed sumoylation and IFN induction. The integration of two distinct functions within a single ‘dead-man's switch’ provides robust self-insurance of the anti-viral MORC3 function. Employing a single protein to repress both viral gene expression and the IFNB1 locus may provide a significant barrier against viruses that seek to selectively escape the repression of viral genes without also triggering IFNB1 expression. It is conceivable that MORC3 exerts its two different functions by executing one unifying molecular activity, namely, transcriptional repression. Various repressive activities have been proposed for the MORC gene family, including DNA methylation, H3K9-methylation, H3.3 incorporation and DNA compaction. The identification of the MRE as a key downstream component required for IFNB1 expression in MORC3-deficient cells is interesting. Loss of the MRE is the only known way to eliminate IFNB1 expression after activation of the MORC3 pathway, but importantly, loss of the MRE does not affect canonical IFNB1-inducing pathways that depend on IRF3/7. Thus, MRE mutant cells can be used to assess whether a specific intervention (e.g., MORC3 inhibitor) is affecting the MORC3 pathway.

Although the MORC3 pathway is distinct from known PRR-mediated pathways of IFN induction, the strategy of self-guarded anti-viral proteins may be more common than has been previously appreciated. The strong selective pressure applied by host-pathogen arms races may select for self-guarded pathways to force a pathogen to “pick its poison”: either the pathogen is restricted by a primary anti-viral activity, or if the pathogen interferes with this activity, a secondary restrictive anti-viral activity is unleashed. Because self-guarding fundamentally involves negative regulation, many examples of self-guarded anti-viral proteins may be hiding in plain sight as negative regulators. Indeed, it is noteworthy that many of the negative regulators of type I IFNs that have been previously described, e.g., ADAR1, SAMHD1, TREX1, RNASEH or ISG15/USP18, have known or speculated intrinsic anti-viral functions, i.e., RNA deamination, nucleotide degradation, DNA degradation, RNA-DNA hybrid degradation, or ISGylation, respectively. Negative regulation of IFN by these enzymes may have evolved to guard their intrinsic anti-viral activity. It is also noteworthy that immune pathways with a primary anti-pathogen function, such as MHC-I or TNFR complex I, have secondary roles in inhibiting other innate immune responses, i.e., NK-cell-mediated cytotoxicity and TNFR complex II, respectively. Negative regulators of NK cells and TNF signaling are also targeted by pathogen effectors. Therefore, self-guarded bi-functional pathways might be a common feature of multi-layered innate immune systems of animals.

As disclosed herein, MORC3 inhibits expression of the interferon beta gene, IFNB1, and loss of MORC3 activity activates a Type I interferon induction pathway that is independent of pattern-recognition-receptors (PRRs).

Therefore, in some embodiments, the present invention provides methods for modulating an interferon response in a subject which comprises modifying the activity of MORC3 in the subject. In some embodiments, the activity of MORC3 is inhibited or reduced. In some embodiments, the activity of MORC3 is enhanced, increased, or stabilized. In some embodiments, MORC3 activity is modified in combination with administration of one or more immunomodulators in the art.

The activity of MORC3 in a subject may be inhibited or reduced by, e.g., knock-out or knock-down of the MORC3 gene in the subject, administering the subject an siRNA that inhibits or reduces expression of MORC3, administering the subject a MORC3 inhibitor, etc. The activity of MORC3 in a subject may be enhanced or increased by, e.g., increasing the copy number of MORC3 in the subject, administering MORC3 to the subject, etc.

In some embodiments, MORC3 activity is modified in a subject in need thereof. A subject “in need of” modification of MORC3 activity is one who will likely benefit from an increase or decrease of interferon. In some embodiments, the subject is one who suffers from an abnormality in an interferon induction signaling pathway, e.g., RIG-I pathway, TRIF pathway, and IRF7 pathway. In some embodiments, the subject is in need of an increase of interferon. In some embodiments, the subject is in need of a decrease of interferon.

In some embodiments, MORC3 activity is inhibited or reduced:
to induce anti-tumor immune response,
to induce anti-viral immune response,
to treat an auto-immune disease or disorder, or
to act as an adjuvant for, e.g., a vaccine.

In some embodiments, MORC3 activity is enhanced, increased, or stabilized to treat an inflammatory disease or disorder involving IFNB1 expression, a disease caused by hyper-IFN expression, systemic lupus erythematosus (SLE), rheumatoid arthritis, multiple sclerosis (MS), type I diabetes (T1D), and Sjögren's syndrome, myositis, etc.

In some embodiments, MORC3 activity is enhanced, increased, or stabilized to treat a bacterial or viral infection.

Methods in the art may be used to identify small molecule inhibitors of MORC3 expression and/or activity. In some embodiments, the small molecule inhibitors block the ATPase activity of MORC3.

In some embodiments, cells lacking the signaling components such as TBK1/IKKE, IKKα/b, and/or IRF3/7, as described herein, are treated with a candidate compound to determine whether the candidate compound stimulates or inhibits interferon production, and thereby indicates whether the candidate compound modulates MORC3 activity.

A compound that modulates MORC3 activity may be optimized to improve its pharmacokinetics using methods in the art and/or targeted to a tissue of interest, e.g., conjugated to a carrier molecule.

As used herein, an “interferon disorder” refers to a disease, infection, or disorder that results in and/or involves an abnormally high or an abnormally low amount of interferon production. In some embodiments, the interferon disorder is an IFNB1 Disorder. As used herein, an “IFNB1 Disorder” refers to diseases and disorders that are, directly or indirectly, caused by abnormal interferon beta (IFNB1) activity, e.g., abnormally high or abnormally low levels of interferon as compared to normal control subjects.

As used herein, a “IFN Excess Disorder” refers to diseases and disorders that, directly or indirectly, cause or result in abnormally high levels of interferon as compared to normal control subjects. IFN Excess Disorders also include abnormally high levels of interferon resulting from infections and therapeutic interventions. Examples of IFN Excess Disorders include autoimmune and inflammatory diseases such as rheumatoid arthritis, psoriasis, vitiligo, hypothyroidism, hyperthyroidism, idiopathic thrombocytopenic purpura, autoimmune hemolytic anemia, myasthenia gravis, Addison disease, celiac disease, polymyositis, superimposed autoimmune hepatitis, and the like. Examples of autoimmune and inflammatory diseases include multiple sclerosis, primary progressive multiple sclerosis, and relapsing-remitting multiple sclerosis.

As used herein, a “IFN Deficiency Disorder” refers to diseases and disorders that, directly or indirectly, cause or result in abnormally low levels of interferon as compared to normal control subjects; and diseases, infections, and/or symptoms that are treatable by administration of interferon. IFN Deficiency Disorders also include abnormally low levels of interferon resulting from infections and therapeutic interventions. Examples of IFN Deficiency Disorders include viral infections (e.g., as herpes, hepatitis, a coronavirus infection (e.g., COVID 19), etc.), cancers (e.g., leukemias, lymphomas, melanomas, sarcomas, adenocarcinomas, etc.), and the like.

As used herein, “MORC3 therapeutic agents” refer to compounds and biomolecules that inhibit, reduce, increase, enhance, or stabilize MORC3 expression and/or activity. MORC3 therapeutic agents include MORC3 inhibitors (e.g., siRNAs, small molecules, ATPase inhibitors (e.g., small molecule ATPase inhibitors), etc.) and MORC3 activators. In some embodiments, the MORC3 therapeutic agent is a small molecule, a protein, or a nucleic acid. In some embodiments, the MORC3 therapeutic agent is an antibody, fusion protein, degrader, shRNA, siRNA, microRNA, asymmetric interfering RNA, antisense molecule, 1hRNA, miRNA embedded shRNA, or small internally segmented RNA. In some embodiments, the MORC3 therapeutic agent is a MORC3 protein or a MORC3 fusion protein. In some embodiments, the MORC3 therapeutic agent comprises an ICP0 protein. In some embodiments, the MORC3 therapeutic agent comprises shRNA, siRNA, microRNA, asymmetric interfering RNA, antisense molecule, 1hRNA, miRNA embedded shRNA, or small internally segmented RNA. the MORC3 therapeutic agent is an siRNA, an ATPase inhibitor (e.g., a small molecule ATPase inhibitor), a MORC3 protein having at least about 90% sequence identity to SEQ ID NO: 1, or an ICP0 protein having at least about 95% sequence identity to SEQ ID NO: 2. Such MORC3 inhibitors and activators include those that bind MORC3 and thereby results in the autoinhibited conformation and the activated conformation, respectively. See, e.g., Zhang et al. (2019) PNAS USA 116(13):6111-6119. In some embodiments, the ATPase inhibitor is (−)-Blebbistatin, Bafilomycin A1 (Baf-A1), BHQ, Brefeldin A, BRITE338733, BTB06584, Bufalin, CB-5083, Dexlansoprazole, Digoxin (NSC 95100), Ilaprazole, Ilaprazole sodium, KM91104, Lansoprazole, ML241 hydrochloride, ML367, NEXIUM (esomeprazole magnesium), Oleandrin (PBI-05204), Omeprazole, Ouabain, Pantoprazole, Pantoprazole sodium, Pantoprazole sodium hydrate, Phlorizin, Revaprazan Hydrochloride, Sodium orthovanadate, Tegoprazan, Tenatoprazole, and Thapsigargin. In some embodiments, the siRNA is selected from the group consisting of hsa-miR-3148, hsa-miR-144-3p, hsa-miR-101-3p, hsa-miR-9902, hsa-miR-367-3p, hsa-miR-3059-3p, hsa-miR-92b-3p, hsa-miR-25-3p, hsa-miR-92a-3p, hsa-miR-363-3p, hsa-miR-32-5p, hsa-miR-570-5p, hsa-miR-548ai, hsa-miR-548ba, hsa-miR-548ag, hsa-miR-6124, hsa-miR-205-3p, hsa-miR-545-5p, hsa-miR-4742-5p, hsa-miR-3908, hsa-miR-153-3p, hsa-miR-3121-3p, hsa-miR-5700, hsa-miR-200b-5p, hsa-miR-7852-3p, hsa-miR-200a-5p, hsa-miR-4262, hsa-miR-6833-5p, hsa-miR-8073, hsa-miR-221-5p, hsa-miR-548m, hsa-miR-3190-3p, hsa-miR-548c-3p, hsa-miR-3145-3p, hsa-miR-12124, hsa-miR-548aj-3p, hsa-miR-548x-3p, hsa-miR-4652-3p, hsa-miR-513a-5p, hsa-miR-218-2-3p, hsa-miR-4729, hsa-miR-2054, hsa-miR-181b-5p, hsa-miR-181c-5p, hsa-miR-181d-5p, hsa-miR-181a-5p, hsa-miR-95-5p, hsa-miR-190a-3p, hsa-miR-5582-3p, hsa-miR-584-5p, hsa-miR-542-3p, hsa-miR-450a-1-3p, hsa-miR-548j-3p, hsa-miR-548 am-3p, hsa-miR-548bb-3p, hsa-miR-548ac, hsa-miR-548aq-3p, hsa-miR-548d-3p, hsa-miR-548ah-3p, hsa-miR-548ae-3p, hsa-miR-548 h-3p, hsa-miR-548z, hsa-miR-7154-5p, hsa-miR-27a-5p, hsa-miR-4275, hsa-miR-1185-2-3p, hsa-miR-1185-1-3p, hsa-miR-622, hsa-miR-4699-3p, hsa-miR-6808-3p, hsa-miR-4719, hsa-miR-3180-5p, hsa-miR-3978, hsa-miR-3163, hsa-miR-4718, hsa-miR-4748, hsa-miR-4464, hsa-miR-887-3p, hsa-let-7f-2-3p, hsa-miR-5696, hsa-miR-3657, hsa-miR-4669, hsa-miR-758-3p, hsa-miR-3649, hsa-miR-5011-5p, hsa-miR-513a-3p, hsa-miR-513c-3p, hsa-miR-3606-3p, hsa-miR-7849-3p, hsa-miR-374b-5p, hsa-miR-4703-5p, hsa-miR-374a-5p, hsa-miR-4423-5p, hsa-miR-4753-3p, hsa-miR-1284, hsa-miR-2110, hsa-miR-4422, hsa-miR-3973, hsa-miR-3942-5p, hsa-miR-367-5p, hsa-miR-183-5p, hsa-miR-8084, hsa-miR-155-5p, hsa-miR-7973, hsa-miR-30a-3p, hsa-miR-30d-3p, hsa-let-7a-3p, hsa-miR-450a-2-3p, hsa-let-7b-3p, hsa-miR-30e-3p, hsa-let-7f-1-3p, hsa-miR-98-3p, hsa-miR-152-5p, hsa-miR-6735-5p, hsa-miR-7843-5p, hsa-miR-7161-5p, hsa-miR-4′761-3p, hsa-miR-6879-5p, hsa-miR-551b-5p, hsa-miR-4632-5p, hsa-miR-944, hsa-miR-649, hsa-miR-4436b-3p, hsa-miR-5087, hsa-miR-4700-3p, hsa-miR-1323, hsa-miR-513b-5p, hsa-miR-651-3p, hsa-miR-6796-3p, hsa-miR-182-3p, hsa-miR-597-3p, hsa-miR-5000-3p, hsa-miR-548u, hsa-miR-6787-3p, hsa-miR-4752, and hsa-miR-216a-5p.

As used herein, the term “sample” is used in its broadest sense and includes specimens and cultures obtained from any source, as well as biological samples and environmental samples. Biological samples may be obtained from animals (including humans) and encompass fluids, solids, tissues, and gases. Biological samples include blood products, such as plasma, serum, and the like. A biological sample can be obtained from a subject using methods in the art. A sample to be analyzed using one or more methods described herein can be either an initial unprocessed sample taken from a subject or a subsequently processed, e.g., partially purified, diluted, concentrated, fluidized, pretreated with a reagent (e.g., protease inhibitor, anti-coagulant, etc.), and the like. In some embodiments, the sample is a blood sample. In some embodiments, the blood sample is a whole blood sample, a serum sample, or a plasma sample. In some embodiments, the sample may be processed, e.g., condensed, diluted, partially purified, and the like. In some embodiments, the sample is pretreated with a reagent, e.g., a protease inhibitor. In some embodiments, two or more samples are collected at different time intervals to assess any difference in the amount of the analyte of interest, the progression of a disease or disorder, or the efficacy of a treatment. The test sample is then contacted with a capture reagent and, if the analyte is present, a conjugate between the analyte and the capture reagent is formed and is detected and/or measured with a detection reagent.

### Kits

In some embodiments, the present invention provides kits for assaying MORC3, in a sample, e.g., a biological sample from a subject. In some embodiments, the kits comprise one or more reagents, e.g., blocking buffers, assay buffers, diluents, wash solutions, etc., for assaying the MORC3. In some embodiments, the kits comprise additional components such as interpretive information, control samples, reference levels, and standards.

In some embodiments, the present invention provides kits comprising one or more MORC3 therapeutic agents, optionally in a composition or in combination with one or more supplementary agents, packaged together with one or more reagents or drug delivery devices for preventing, inhibiting, reducing, or treating interferon disorder in a subject. In some embodiments, the kits comprise the one or more MORC3 therapeutic agents, optionally in one or more unit dosage forms, packaged together as a pack and/or in drug delivery device, e.g., a pre-filled syringe.

In some embodiments, the kits include a carrier, package, or container that may be compartmentalized to receive one or more containers, such as vials, tubes, and the like. In some embodiments, the kits optionally include an identifying description or label or instructions relating to its use. In some embodiments, the kits include information prescribed by a governmental agency that regulates the manufacture, use, or sale of compounds and compositions as contemplated herein.

### Diagnostic and Prognostic Applications

The methods and kits as contemplated herein may be used in the evaluation of an interferon disorder, such as a IFN Deficiency Disorder or a IFN Excess Disorder. The methods and kits may be used to monitor the progress of such a disease, assess the efficacy of a treatment for the disease, and/or identify patients suitable for a given treatment in a subject. The methods and kits may be used to diagnose a subject as having a interferon disorder and/or provide the subject with a prognosis.

In some embodiments, the methods and kits may be used to determine whether a subject exhibits a level of MORC3 that is low or high as compared to a control. In some embodiments, the control is a sample from a normal, healthy subject. In some embodiments, the control is a pooled sample from a plurality of normal, healthy subjects. In some embodiments, the control is a given reference level. The abnormal level may then be used to diagnose the subject as suffering from an interferon disorder.

A subject identified as having a low level or a high level of MORC3 may be subjected to a suitable treatment. For example, a subject identified as having a high level of MORC3 or diagnosed as suffering from an IFN Excess Disorder may be treated with one or more MORC3 therapeutic agents. As another example, a subject identified as having a low level of MORC3 or diagnosed as suffering from a IFN Deficiency Disorder may be treated with one or more MORC3 therapeutic agents.

In some embodiments, the methods and kits may be used to monitor the efficacy of treatment with a given therapeutic, e.g., a MORC3 therapeutic agent, that modulates the level of MORC3 produced in the subject and the dosage of the given therapeutic may be adjusted accordingly.

### Non-Clinical Applications

In some embodiments, the methods and kits may be used for research purposes. For example, the methods and kits may be used to identify diseases that are caused by abnormal levels of MORC3 and/or identify diseases that result in abnormal levels of MORC3. In some embodiments, the methods and kits may be used to study mechanisms, e.g., mechanisms and pathways involving MORC3. In some embodiments, the methods and kits may be used to develop and screen for therapeutics that increase or decrease levels of MORC3 in subjects.

### Compositions

Compositions, including pharmaceutical compositions, comprising, consisting essentially of, or consisting of one or more MORC3 therapeutic agents are contemplated herein. The term “pharmaceutical composition” refers to a composition suitable for pharmaceutical use in a subject. A composition generally comprises an effective amount of an active agent and a diluent and/or carrier. A pharmaceutical composition generally comprises a therapeutically effective amount of an active agent and a pharmaceutically acceptable carrier. In addition to the one or more MORC3 therapeutic agents, pharmaceutical compositions may include one or more supplementary agents. Examples of suitable supplementary agents include immunomodulatory agents, interferon, and the like.

As used herein, an “effective amount” refers to a dosage or amount sufficient to produce a desired result. The desired result may comprise an objective or subjective change as compared to a control in, for example, in vitro assays, and other laboratory experiments. As used herein, a “therapeutically effective amount” refers to an amount that may be used to treat, prevent, or inhibit a given disease or condition in a subject as compared to a control, such as a placebo. Again, the skilled artisan will appreciate that certain factors may influence the amount required to effectively treat a subject, including the degree of the condition or symptom to be treated, previous treatments, the general health and age of the subject, and the like. Nevertheless, effective amounts and therapeutically effective amounts may be readily determined by methods in the art.

The one or more MORC3 therapeutic agents may be administered, preferably in the form of pharmaceutical compositions, to a subject. Preferably the subject is mammalian, more preferably, the subject is human. Preferred pharmaceutical compositions are those comprising at least one MORC3 therapeutic agent in a therapeutically effective amount and a pharmaceutically acceptable vehicle. It should be noted that treatment of a subject with a therapeutically effective amount may be administered as a single dose or as a series of several doses. The dosages used for treatment may increase or decrease over the course of a given treatment. Optimal dosages for a given set of conditions may be ascertained by those skilled in the art using dosage-determination tests and/or diagnostic assays in the art. Dosage-determination tests and/or diagnostic assays may be used to monitor and adjust dosages during the course of treatment.

Pharmaceutical compositions may be formulated for the intended route of delivery, including intravenous, intramuscular, intra peritoneal, subcutaneous, intraocular, intrathecal, intraarticular, intrasynovial, cisternal, intrahepatic, intralesional injection, intracranial injection, infusion, and/or inhaled routes of administration using methods known in the art. Pharmaceutical compositions may include one or more of the following: pH buffered solutions, adjuvants (e.g., preservatives, wetting agents, emulsifying agents, and dispersing agents), liposomal formulations, nanoparticles, dispersions, suspensions, or emulsions, as well as sterile powders for reconstitution into sterile injectable solutions or dispersions. The compositions and formulations may be optimized for increased stability and efficacy using methods in the art. See, e.g., Carra et al., (2007) Vaccine 25:4149-4158.

The compositions may be administered to a subject by any suitable route including oral, transdermal, subcutaneous, intranasal, inhalation, intramuscular, and intravascular administration. It will be appreciated that the preferred route of administration and pharmaceutical formulation will vary with the condition and age of the subject, the nature of the condition to be treated, the therapeutic effect desired, and the particular MORC3 therapeutic agent used.

As used herein, a “pharmaceutically acceptable vehicle” or “pharmaceutically acceptable carrier” are used interchangeably and refer to solvents, dispersion media, coatings, antibacterial and antifungal agents, isotonic and absorption delaying agents, and the like, that are compatible with pharmaceutical administration and comply with the applicable standards and regulations, e.g., the pharmacopeial standards set forth in the United States Pharmacopeia and the National Formulary (USP-NF) book, for pharmaceutical administration. Thus, for example, unsterile water is excluded as a pharmaceutically acceptable carrier for, at least, intravenous administration. Pharmaceutically acceptable vehicles include those known in the art. See, e.g., Remington: The Science and Practice of Pharmacy 20th ed (2000) Lippincott Williams & Wilkins, Baltimore, MD.

The pharmaceutical compositions may be provided in dosage unit forms. As used herein, a “dosage unit form” refers to physically discrete units suited as unitary dosages for the subject to be treated; each unit containing a predetermined quantity of the one or more MORC3 therapeutic agent calculated to produce the desired therapeutic effect in association with the required pharmaceutically acceptable carrier. The specification for the dosage unit forms of the invention are dictated by and directly dependent on the unique characteristics of the given MORC3 therapeutic agent and desired therapeutic effect to be achieved, and the limitations inherent in the art of compounding such an active compound for the treatment of individuals.

Toxicity and therapeutic efficacy of MORC3 therapeutic agents according to the instant invention and compositions thereof can be determined using cell cultures and/or experimental animals and pharmaceutical procedures in the art. For example, one may determine the lethal dose, LC₅₀ (the dose expressed as concentration×exposure time that is lethal to 50% of the population) or the LD₅₀ (the dose lethal to 50% of the population), and the ED₅₀ (the dose therapeutically effective in 50% of the population) by methods in the art. The dose ratio between toxic and therapeutic effects is the therapeutic index and it can be expressed as the ratio LD₅₀/ED₅₀. MORC3 therapeutic agents which exhibit large therapeutic indices are preferred. While MORC3 therapeutic agents that result in toxic side-effects may be used, care should be taken to design a delivery system that targets such compounds to the site of treatment to minimize potential damage to uninfected cells and, thereby, reduce side-effects.

The data obtained from the cell culture assays and animal studies can be used in formulating a range of dosages for use in humans. Preferred dosages provide a range of circulating concentrations that include the ED₅₀ with little or no toxicity. The dosage may vary depending upon the dosage form employed and the route of administration utilized. Therapeutically effective amounts and dosages of one or more MORC3 therapeutic agents can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. Additionally, a dosage suitable for a given subject can be determined by an attending physician or qualified medical practitioner, based on various clinical factors.

The following examples are intended to illustrate but not to limit the invention.

### Examples

### Cell Culture

BLaER1, U937 and THP-1 cells were cultured in RPMI Medium 1640 supplemented with L-glutamine, sodium pyruvate, 100 U/ml penicillin-streptomycin (Thermo Fisher) and 10% (v/v) FCS (Omega Scientific). HEK293T, U2OS and HCT116 cells were cultivated in DMEM Medium (Thermo Fisher) containing the same supplements. 1.4 million BLaER1 cells per well of a 6-well plate were trans-differentiated into monocytes for 5-6 days in medium containing 10 ng/ml of hrIL-3, 10 ng/ml hr-CSF-1 (M-CSF) (both PeproTech) and 100 nM β-Estradiol (Sigma-Aldrich) as previously described. 1.4 million THP-1 and U937 cells per well of a 6-well plate were differentiated overnight with 100 ng/ml PMA (Sigma-Aldrich). STING-deficient and corresponding control THP1 and U937 cells were a gift from Dan Stetson (University of Washington). BLaER1 cells were a gift from Thomas Graf (CRG, Barcelona, Spain) and Veit Hornung (LMU Munich, Germany). U2OS cells were a gift from Robert Tjian and Xavier Darzacq (UC Berkeley). THP1 cells were from ATCC. U937 cells were from the UC Berkeley Cell Culture Facility. HCT116 cells were a gift from David Raulet (UC Berkeley).

### Cell Stimulation

For activation of the cGAS-STING pathway, 3.2 μg of UltraPure™ Salmon Sperm DNA (Thermo Fisher) or 3.2 μg of 2′3′ cGAMP (Invivogen) was complexed with 8 11.1 Lipofectamine 2000 (Thermo Fisher) according to the manufacturer's protocol in Opti-MEM Reduced Serum Media (Thermo Fisher) and added to 1.4 million cells per well of a 6-well plate for 3 h or the indicated time. PRRs were activated with 200 ng/ml LPS-EB ultrapure from E. coli O111:B4 (Invivogen) or 500 ng/ml R848 (Invivogen). For activation of doxycycline-inducible trans-gene expression, cells were stimulated with 1 μg/ml doxycycline hyclate (Sigma-Aldrich) for 24 h.

### HSV-1 Infection

BACs of ΔICP0 HSV-1 and corresponding WT strain were a gift from Bernard Roizman (University of Chicago). BAC DNA was prepared from a mono-clonal transformant and transfected into U2OS cells using Lipofectamine 2000 (Thermo Fisher). Virus was propagated, harvested and frozen as described. Viral progeny were titered from cell-free supernatants by TCID50 using 8 replicates per dilution. U2OS cells were used for titering if not otherwise indicated, and FFU/ml was calculated by the Spearman & Karber algorithm. Myeloid cells were infected by adsorbing virus of appropriate MOI in FCS free RPMI Medium 1640 for 1 h. Subsequently, medium was changed to complete RPMI Medium 1640. For analysis of viral progeny in the supernatant, cells were subsequently washed three times with warm PBS and resuspended in RPMI. For all other experiments, medium was directly changed to RPMI without a PBS wash.

### Quantification of Gene Expression

Gene expression was quantified by quantitative PCR with reverse transcription (RT-qPCR). RNA was isolated with E.Z.N.A. Total RNA kit I (Omegabiotek) and 0.5-1 μg RNA was treated with RQ1 RNase-free DNase (Promega) in presence of RNasin plus ribonuclease inhibitor (Promega). RNA was reverse transcribed with Superscript III reverse transcriptase (Invitrogen). SYBRGreen dye (Thermo Fisher Scientific) was used for quantitative PCR assays and analyzed with a real-time PCR system (StepOnePlus; Applied Biosystems). All gene expression values were normalized to GAPDH and are depicted as 2\hat{ }-ΔCt (Ct_{target}−Ct_{GAPDH}). Primers used were:

RSAD2.fwd (SEQ ID NO: 4):
CAACTACAAATGCGGCTTCT
RSAD2.rev (SEQ ID NO: 5):
ATCTTCTCCATACCAGCTTCC
CXCL10.fwd (SEQ ID NO: 6):
TCTGAATCCAGAATCGAAGG
CXCL10.rev (SEQ ID NO: 7):
CTCTGTGTGGTCCATCCTTG
GAPDH.fwd (SEQ ID NO: 8):
GAGTCAACGGATTTGGTCGT
GAPDH.rev (SEQ ID NO: 9):
GACAAGCTTCCCGTTCTCAG
IFNB1.fwd (SEQ ID NO: 10):
CAGCATCTGCTGGTTGAAGA
IFNB1.rev (SEQ ID NO: 11):
CATTACCTGAAGGCCAAGGA
MLLT3.fwd (SEQ ID NO: 12):
GAGCACAGTAACATACAGCA
MLLT3.rev (SEQ ID NO: 13):
GGCAAAATGAAACCAGCATA

### Immunoblotting

Whole cell lysates were prepared by lysing cells in 50 mM Tris pH 7.4, 50 mM NaCl, 2 mM MgCl₂, 0.5% NP40, 25 U/ml Benzonase® Nuclease (Millipore Sigma) and Complete Mini EDTA-free Protease Inhibitor (Roche) for 20 min on ice. Laemmli buffer was added to a final concentration of 1× and lysates were boiled at 95° C. for 10 minutes. Proteins were separated with denaturing PAGE and transferred to Immobilon-FL PVDF membranes (Millipore Sigma). Membranes were blocked with Li-Cor Odyssey blocking buffer. Primary antibodies were added and immunoblots incubated overnight. Primary antibodies used were anti-β-Actin (C4) (Santa Cruz, sc-47778), anti-HSV-1 ICP4 (H943) (Santa Cruz, sc-69809), anti-HSV-1 ICP0 (11060) (Santa Cruz, sc-53070), anti-TBK1 (D1B4) (Cell Signaling, #3504), anti-IKKε (Cell Signaling, #2690), anti-IRF-3 (D83B9) (Cell Signaling, #4302), anti-IRF-7 (Cell Signaling, #4920), anti-MORC3 (NovusBio, NBP1-83036), anti-MORC3 (Proteintech, 24994-1-AP). Appropriate secondary IRDye®-conjugated antibodies (Li-Cor) were used and immunoblots were imaged using the Li-Cor Odyssey platform.

### Quantification of Allele-Specific IFNB1 Expression

To test whether the MRE regulates IFNB1 in cis or in trans, IFNB1 coding sequence with distinct indels were engineered. This enabled us to track which allele is being transcribed upon activation of IFNB1 by amplicon sequencing of IFNB1 cDNA (FIG. “4d). Cytosolic DNA sensing drove bi-allelic activation of IFNB1 regardless of the MRE. Activating the MORC3 pathway with a MORC3-sgRNA drove bi-allelic expression in WT cells and mono-allelic expression of IFNB1 in heterozygous MRE+/′ cells. While the long distance between IFNB1 and the MRE makes it difficult to determine which indel-marked IFNB1 allele is in cis with MRE′, these data strongly suggest that the MRE induces IFNB1 transcription in cis. Specifically, the genome of heterozygous MRE+/′ and corresponding WT (STAT1−/−STAT2−/− Cas9) BLaER1 cells was edited at the IFNB1 locus using the sgRNA GATGAACTTTGACATCCCTGAGG (SEQ ID NO: 14) (protospacer-adjacent motif (PAM) is highlighted in bold) as described in “CRISPR-Cas9-mediated gene targeting” below.

### CRISPR/Cas9 Mediated Gene Targeting

Monoclonal gene deficient BLaER1 cells were generated as follows. Briefly, sgRNAs specific for the indicated genes, were designed to target an early coding exon of the respective gene with minimal off-targets and high on-target activity using ChopChop. U6-sgRNA-CMV-mCherry-T2A-Cas9 plasmids were generated by ligation-independent-cloning as previously described and BlaER1 cells were electroporated using a Biorad GenePulser device. Automated cell sorting was used to collect mCherry positive cells that were cloned by limiting dilution. Monoclonal cell lines were identified, rearranged and duplicated for genotyping using deep sequencing as previously described. Knockout cell clones contained all-allelic frame shift mutations without any wild type reads. Two independent knockout single-cell clones were analyzed per genotype, and one representative clone per genotype is shown. For polyclonal gene targeting, cell lines were transduced with lentiCas9-Blast, a gift from Feng Zhang (Addgene plasmid #52962; http://n2t.net/addgene:52962; RRID: Addgene 52962). sgRNAs were designed as above and cloned into lentiGuide-Puro, a gift from Feng Zhang (Addgene plasmid #52963; http://n2t.net/addgene:52963; RRID: Addgene 52963), using ligation-independent-cloning. Cas9-expressing cells were transduced with indicated sgRNA-encoding lenti-viruses.

sgRNA Target Sites (PAM is Highlighted in Bold):

STING (SEQ ID NO: 15):
GCGGGCCGACCGCATTTGGGAGG
TBK1 (SEQ ID NO: 16):
ACAGTGTATAAACTCCCACATGG
IKBKE (IKKε) (SEQ ID NO: 17):
TGCATCGCGACATCAAGCCGGGG
CHUK (IKKα) (SEQ ID NO: 18):
TAGTTTAGTAGTAGAACCCATGG
IKBKB (IKKβ) (SEQ ID NO: 19):
GCCATGGAGTACTGCCAAGGAGG
IRF7 (SEQ ID NO: 20):
CCGAGCTGCACGTTCCTATACGG
IRF3 (SEQ ID NO: 21):
GTTACTGGGTAACATGGTGTTGG
TRIM33 (SEQ ID NO: 22):
GTTATGAACTTCACAAATTGGG
VMP1 (SEQ ID NO: 23):
GAACTGCCAGTTTGGCCCGGG
UBA3 (SEQ ID NO: 24):
GGCCTAAGGAGCAGCCTTTTGG
NAE1 (SEQ ID NO: 25):
GAATTAAATAGCGATGTCTCTGG
NF1 (SEQ ID NO: 26):
GCTGGTTTCCTTCACGACAGG
USP18 (SEQ ID NO: 27):
GGCACAGTCAACGCAAATCAAGG
NFIC (SEQ ID NO: 28):
GCTGCTGGGCGAGAAGCCCGAGG
EFR3A (SEQ ID NO: 29):
GATTGCTATGGAGGCACTGG
PTPN1 (SEQ ID NO: 30):
GAGCAGATCGACAAGTCCGGG
MORC3 (SEQ ID NO: 31):
GCTGATACTGAGATACCATATGG
TAF5L (SEQ ID NO: 32):
GCTGCTCAATGACATCCTTCTGG
RNF7 (SEQ ID NO: 33):
GGCCATGTGGAGCTGGGACGTGG
PI4KA (SEQ ID NO: 34):
GGGATAGCATACTTGCAAAGG
IFNAR1 (SEQ ID NO: 35):
GTACATTGTATAAAGACCACAGG
IFNAR2 (SEQ ID NO: 36):
TGAGTGGAGAAGCACACACGAGG
STAT1 (SEQ ID NO: 37):
CAGGAGGTCATGAAAACGGATGG
STAT2 (SEQ ID NO: 38):
ATCATCTCAGCCAACTGGGTAGG
IFNB1 (SEQ ID NO: 39):
GATGAACTTTGACATCCCTGAGG
scramble (SEQ ID NO: 40):
GCTGCTCCCTAACAGGACGC

Cytokine Quantification Cytokine secretion was quantified by ELISA of cell-free supernatants after stimulations (IFNβ: R&D, DY814-05; IP-10: BD, 550926).

### Lenti-/Retro-Viral Transduction

Lenti- and retro-virus was produced in HEK293T cells. 4.5 million cells were plated per 10 cm dish and transfected with 5 μg of transfer vector, 3.75 μg of packaging vector (pd8.9 for lenti- and pGAGPOL for retro-virus) and 1.5 μg pVSVG using 30.75 μg PEI-MAX (Polysciences, 24765-1). 12 h after transfection the medium was replaced with DMEM medium containing 30% (v/v) FCS. After 24-36 h, viral supernatants were harvested, centrifugated at 1000×g for 10 min, and filtered through a 0.45 μm filter. Cells were culture for 48 h after transduction, prior to selection with puromycin or Blasticidine S hydrochloride (both Sigma-Aldrich).

### Ectopic Gene Expression

A doxycycline-inducible lenti-virus system was used for ectopic gene expression as previously described. Codon-optimized constructs for HSV-1-ICP0 and HA-Adenovirus5-E4ORF3 were synthesized by Integrated DNA Technologies and cloned into pLIP. ICP0 variants were generated by overlap-extension PCR.

### IFNB1-Reporter

The 1 kb upstream of the transcription start site of huma IFNB1 (hg38 chr9:21077923-21078922) was synthesized by Integrated DNA Technologies and cloned in front of a luciferase reporter from Gaussia princeps into a retro-viral transfer vector in opposite direction to the 5′LTR. BLaER1 cells were transduced and sorted for reporter integration.

### Flow Cytometry

BLaER1 were harvested for flow cytometry, fixed and permeabilized using eBioscience™ IC Fixation Buffer and eBioscience™ Permeabilization Buffer (both Thermo Fisher) according to the provider's protocol. Cells were incubated for 1 h with PE-Anti-Viperin (Clone MaP.VIP; BD, 565196) and analyzed using a BD LSRFortessa™ Flow Cytometer. If indicated, cells were sorted on a BD FACSAria™ Fusion Cell Sorter. For analysis of HSV-1 infection BLaER1 were collected for flow cytometry, fixed in 4% PFA (Electron Microscopy Sciences) for 15 (min at room temperature and washed 3 times with PBS. Cells were permeabilized with HSV-1 stain buffer (PBS, 10% FCS, 1 mM EDTA and 0.1% saponin) for 30 min on ice, incubated with Human BD Fc Block (BD) for 30 min on ice and stained with anti-HSV-1 antibody (ab9533, Abcam) at 1:100 dilution for 1 hon ice. After 3 washes with HSV-1 staining buffer, cells were stained with anti-rabbit secondary antibody (A-21244, Thermo Fisher Scientific) at 1:2,000 dilution in HSV-1 stain buffer, washed 4 times in HSV-1 stain buffer and analysed using a BD LSRFortessa Flow Cytometer.

### CRISPR-Screen

Monoclonal Cas9-expressing BLaER1 cells were re-selected with blasticidine and 9 million cells were transduced in four biological replicates with a pooled Human CRISPR Knockout library at an MOI of approximately 0.3. The library was a gift from Michael Bassik (Addgene #101926, 101927, 101928, 101929, 101930, 101931, 101932, 101933, 101934). Two days after transduction, cells were selected with puromycin for 3 days and trans-differentiated. Cells were stained for Viperin expression. Per biological replicate, 46-80×10³ cells with increased spontaneous Viperin expression were sorted (FIG. 9-a) into direct lysis buffer (0.2 mg/ml proteinase K, 1 mM CaCl₂, 3 mM MgCl₂, 1 mM EDTA, 1% Triton X-100, 10 mM Tris pH 7.5). As control, sgRNA positive cells were sorted irrespectively of their Viperin expression. The reactions were incubated at 65° C. for 10 min and at 95° C. for 15 min. The integrated sgRNA cassette was amplified using a nested PCR approach with Phusion DNA Polymerase (Thermo Fisher) with 4 technical replicates per biological replicate. Primers for the first level utilized a mix of staggered forward primers:

pMCB320fwd 0 nt stagger (SEQ ID NO: 41):
ACACTCTTTCCCTACACGACGCTCTTCCGATCTCCTTGGAGAACCACCTT
GTTGG
pMCB320fwd 1 nt stagger (SEQ ID NO: 42):
ACACTCTTTCCCTACACGACGCTCTTCCGATCTCCCTTGGAGAACCACCT
TGTTGG
pMCB320fwd 2 nt stagger (SEQ ID NO: 43):
ACACTCTTTCCCTACACGACGCTCTTCCGATCTGCCCTTGGAGAACCACC
TTGTTGG
pMCB320fwd 3 nt stagger (SEQ ID NO: 44):
ACACTCTTTCCCTACACGACGCTCTTCCGATCTAGCCCTTGGAGAACCAC
CTTGTTGG
pMCB320fwd 4 nt stagger (SEQ ID NO: 45):
ACACTCTTTCCCTACACGACGCTCTTCCGATCTCAACCCTTGGAGAACCA
CCTTGTTGG
pMCB320fwd 6 nt stagger (SEQ ID NO: 46):
ACACTCTTTCCCTACACGACGCTCTTCCGATCTTGCACCCCTTGGAGAAC
CACCTTGTTGG
pMCB320fwd 7 nt stagger (SEQ ID NO: 47):
ACACTCTTTCCCTACACGACGCTCTTCCGATCTACGCAACCCTTGGAGAA
CCACCTTGTTGG
pMCB320fwd 8 nt stagger (SEQ ID NO: 48):
ACACTCTTTCCCTACACGACGCTCTTCCGATCTGAAGACCCCCTTGGAGA
ACCACCTTGTTGG
pMCB320rev2 (SEQ ID NO: 49):
TGACTGGAGTTCAGACGTGTGCTCTTCCGATCTTCACCACACACGGCACT
TACCT

Details of the nested PCR approach and primers for the second level PCR have been described. PCR products were sequenced on Illumina HiSeq4000 50SR. Deep sequencing data was analyzed with PinAPL-Py. The recovery of the sgRNA library was suboptimal, probably due to low number of sorted cells. Subsequently, reads from all technical and biological replicates were combined and enriched sgRNAs over control were identified. Modified robust ranking aggregation (RRA) to gene level revealed candidate negative regulators of IFN (Extended Data Table 1). Candidates with a significantly highly ranked sgRNAs (p.adjust <0.01) were validated in an arrayed format (FIG. 2-c).

### RNA-Seq

RNA from 2.8 million trans-differentiated BLaER1 monocytes was isolated using TRIzol™ Reagent (Thermo Fisher) according to the manufacturer's recommendation. DNA was removed with RQ1 RNase-free DNase (Promega) in the presence of RNasin plus Ribonuclease Inhibitor (Promega) and RNA isolated with Agencourt AMPure XP beads (Beckman Coulter). mRNA-seq libraries were prepared by the QB3 Genomics Functional Genomics Laboratory from poly-A-enriched mRNA using a KAPA mRNA HyperPrep Kit (Roche) and sequenced on the Illumina Novaseq S4 150PE. Sequencing quality of fastq files was evaluated with FASTQC and paired end RNA-seq reads were aligned to the reference genome (GRCh38.83) using Bowtie2 with default settings. Transcript and gene counts were quantified using RSEM with the parameter strandedness' set to ‘reverse’ to account for strand-specific library preparation protocol. DeSeq2 was used to identify differentially expressed genes between conditions by building a single DeSeq2 model using counts from RSEM and performed pairwise comparisons of conditions. All genes with a false discovery rate (FDR) below 0.05 were considered to be significantly differential. Log normalized counts from DeSeq2 were used to perform principal component analysis (PCA) shown in FIG. 2-a-FIG. 2-g and to generate heatmaps. Gene enrichment analysis was done with the R package clusterProfiler. For each enrichment analysis, the foreground was assigned to be the set of genes with significant increased expression in the condition being evaluated. The background was set to the set of all genes that have mean counts greater than 1 for the condition being evaluated. Gene enrichment sets were downloaded from the Molecular Signatures Database (MSigDB).

### ATAC-Seq

ATAC-seq was performed using methods in the art. 50,000 BLaER1 monocytes were washed with PBS and lysed in 50 (μl of cold lysis buffer (10 mM Tris-HCl, pH 7.4, 10 mM NaCl, 3 mM MgCl2, 0.1% IGEPAL CA-630). Nuclei were collected by centrifugation and resuspended in 50 (μl TD Buffer (Illumina, FC-121-1030) with 2.5 μl Tn5 Transposase (Illumina, FC-121-1030). The reaction was incubated for 30 (min at 37(° C. and DNA was isolated with a MinElute Kit (Qiagen). Transposed DNA fragments were amplified to reach 30% of maximal amplification using Ad1 and Ad2 primer and sequenced on an Illumina Nova-Seq SP 50PE.

Primers:

Ad1 (SEQ ID NO: 50):
AATGATACGGCGACCACCGAGATCTACACTCGTCGGCAGCGTCAGATGTG
Ad2.1 (SEQ ID NO: 51):
CAAGCAGAAGACGGCATACGAGATTCGCCTTAGTCTCGTGGGCTCGGAGA
TGT
Ad2.2 (SEQ ID NO: 52):
CAAGCAGAAGACGGCATACGAGATCTAGTACGGTCTCGTGGGCTCGGAGA
TGT
Ad2.3 (SEQ ID NO: 53):
CAAGCAGAAGACGGCATACGAGATTTCTGCCTGTCTCGTGGGCTCGGAGA
TGT
Ad2.4 (SEQ ID NO: 54):
CAAGCAGAAGACGGCATACGAGATGCTCAGGAGTCTCGTGGGCTCGGAGA
TGT
Ad2.5 (SEQ ID NO: 55):
CAAGCAGAAGACGGCATACGAGATAGGAGTCCGTCTCGTGGGCTCGGAGA
TGT
Ad2.6 (SEQ ID NO: 56):
CAAGCAGAAGACGGCATACGAGATCATGCCTAGTCTCGTGGGCTCGGAGA
TGT

FASTQC version 0.11.539 was used to assess quality of ATAC-seq reads. Adapter sequences were removed using Cutadapt version 2.1046 using a default error rate of 0.1. Reads shorter than 5 were discarded. Reads were aligned to the hg19 reference genome using bowtie 2 version 2.3.240 and discordant alignments were removed. Reads with mapping quality less than 30 were removed using SAMtools version 1.3.147 and duplicates were removed using Picard Tools version 2.5.0 (broadinstitute.github.io/picard/). Additionally, regions overlapping black list regions, identified from the ENCODE consortium, were removed. Blacklist regions were downloaded from ENCODE (accession ENCFF000KJP). ATAC-seq reads aligned to the positive strand were shifted +4 bp and reads aligned to the negative strand were shifted −5 bp to adjust read start sites to represent the center of the transposase-binding event. Peaks were called on shifted reads using MACS2 version 2.2.7.150, setting the FDR to 0.05 and the default human genome size. Peaks from all samples were combined by taking the union of all peaks. Counts representing peak strength for all samples were obtained by counting the number of cut sites that overlapped each peak for each sample. DeSeq2 version 1.30.142 was used to identify the differential abundance of cut sites between conditions. All regions with an FDR below 0.05 were marked as significantly differentially accessible. Annotatr was used to gather annotations of ATAC-seq peaks from the hg19 genome. To identify peaks that did not overlap promoters, all ATAC-seq peaks that overlapped at least one annotated promoter were removed.

### Quantification of Expression of ERV Families

RNA-seq reads from IFNAR1^{-/-}IFNAR2^{-/-} mCherry and MORC3^{-/-} IFNAR1^{-/-}IFNAR2^{-/-} BLaER1 monocytes were aligned to the hg38 genome using the STAR aligner with parameter ‘outFilterMultimapNmax’ set to ‘100000000’ to allow for lenient alignment of multimapped reads. RepEnrich2 was used to quantify expression of ERV families. The MAPQ threshold for subsetting uniquely mapping and multi-mapping reads in RepEnrich2 was set to 255. The pre-built repeat annotations for RepEnrich2, available through the RepEnrich2 GitHub was used. Differential expression of ERV counts from RepEnrich2 was analyzed using both the recommended edgeR and DeSeq2 pipelines to identify an overlapping set of high confidence differentially expressed ERVs. All ERVs with an FDR below 0.05 were considered to be significantly differential.

### Gene Targeting in Primary Human Macrophages

Human peripheral blood mononuclear cells (PBMCs) from de-identified donors were obtained from AllCells (Alameda) under donor-informed consent and Alpha IRB approval obtained by AllCells (7000-SOP-045) for the study ‘Non-Mobilized Mononuclear Cell Apheresis Collection from Healthy Donors for the Research Market’. Monocytes were isolated by negative selection (Pan Monocyte Isolation Kit, Miltenyi Biotec) and nucleofected with Cas9 gRNA ribonucleoparticles as previously described55. Alt-R CRISPR RNAs (crRNAs) and Alt-trans-activating CRISPR RNA (tracrRNA) (IDT) were resuspended to 100 μM in nuclease-free duplex buffer (IDT) and annealed at equimolar concentrations for 5 min at 95° C. and 15 min at 20° C. 10 μg Cas9 (IDT Alt-R S.p. Cas9 Nuclease V3) was mixed with 2011 of the crRNA-tracrRNA complex and incubated for 20 min at room temperature before 1 μl of a 4 μM solution of electroporation enhancer (IDT) in nuclease-free duplex buffer (IDT) was added. One-million monocytes in 20 μl P3 nucleofection solution (Lonza) were added to the Cas9-crRNA-tracrRNA complex and nucleofected with a Lonza 4D-Nucleofector (4D-Nucleofector Core Unit, 4D-Nucleofector X Unit) using the settings Buffer P3, CM-137. Cells were immediately resuspended in pre-warmed medium and cultivated for 5 days. Every other day half of the medium was replaced with fresh medium.

crRNA Target Sites:

MORC3 (SEQ ID NO: 57):
GCTGATACTGAGATACCATATGG
scramble (SEQ ID NO: 58):
GCTGCTCCCTAACAGGACGC

Assays for MORC3 Inhibitors that Induce an IFN Response

The following assays may be used to screen for MORC3 inhibitors that induce an IFN response:

BLaER1 human monocytes: Transdifferentiated 70,000 BLaER1 human monocytes are cultivated in RPMI 1640 Medium supplemented with 2 mM L-Glutamine, 1 mM Sodium Pyruvate, 100 U/mL Penicillin, 100 U/mL Streptomycin, 10% (v/v) heat-inactivated FCS, 10 ng/mL hrIL-3, 10 ng/mL hr-M-CSF and 100 nM Estradiol for 5 days at 37° C. and 5% CO₂. The culture media is change to plain RPMI without hrIL-3, hr-M-CSF, and Estradiol. Then a given candidate MORC3 inhibitor is added thereto and incubated for about 24-48 hours at 37° C. and 5% CO₂. Cell-free supernatant is harvested therefrom and an IFNB1 ELISA (R&D, DY814-05) is performed according to the manufacturer's protocol and Type I IFN Bioassay (HEK-Blue™ IFN-α/(3 Cells, Invivogen, hkb-ifnab) against a recombinant protein standard as recommended by the provider and the results are compared to a control. Gene-deficient BLaER1 monocytes (e.g., TBK1⁻/IKKe⁻, IKKa⁻/b⁻, or IRF3⁻/7⁻) may be used to validate that the IFN response induced by a candidate MORC3 inhibitor mirrors the IFN response induced by a genetic MORC3 deficiency.

Primary human monocytes: PBMCs are isolated from peripheral blood of human donors and overlaid with 13 ml of Ficoll solution (Millipore Sigma, GE17-1440-02) and 25 ml of anti-coagulated peripheral blood. The cells are pelletized, e.g., at about 2000 rpm for 20 minutes at 22° C. to result in a pellet of and a white disc on top of the ficoll layer. The disc is aspirated from slightly above with a 10 ml pipet and transferred to a new tube. NaCl is added thereto to give a final volume of 50 ml. The mixture is spun at 450 rcf for 7 min. The pellet is resuspended in 10 ml of erythrocyte lysis solution (e.g., 1 mL BD Pharm Lysis concentrate and 9 ml H₂O). Cells are lysed for 5 min. Then NaCl is added to give a final volume of 50 ml. The mixture is centrifuged at 450 rcf for 7 min. The pellet is resuspended in 50 ml fresh NaCl. The cells are counted and cells to be used are spun down and resuspended in RPMI 1640 Medium supplemented with 2 mM L-Glutamine, 1 mM Sodium Pyruvate, 100 U/mL Penicillin, 100 U/mL Streptomycin, 10% (v/v) heat-inactivated FCS. CD14+ monocytes may be optionally isolated using CD14 MicroBeads, human (Miltenyi, 130-050-201). The cells are plated and the given candidate MORC3 inhibitor is added thereto and then incubated for 24-48 h at 37° C. and 5% CO₂. The cell-free supernatant is harvested and an IFNB1 ELISA (R&D, DY814-05) is performed according to the manufacturer's protocol and Type I IFN Bioassay (HEK-Blue™ IFN-α/β Cells, InvivoGen, hkb-ifnab) against a recombinant protein standard as recommended by the provider and the results are compared to a control.

### Assay for Assessing the ATPase Activity of MORC3

The following is an exemplary protocol for assessing the ATPase activity of MORC3:
1) PCR amplify human MORC3, including the full-length open reading frame, or the ATPase and CW domains, or the ATPase only domain.
2) Clone each PCR product into pET28a such that it has a N-Terminal 6×His Tag attached by a Thrombin site to Morc3.
3) After validating each construct by sequencing, transform into BL21 RIL cells. Inoculate a single colony in 2 mL LB with Kanamycin at a final concentration of 50 μg/ml. Incubate shaking 6-8 hours at 37° C.
4) Add 2 mL culture to 100 mL LB+Kanamycin and Incubate shaking overnight.
5) In the morning add 10 mL of culture to 1L LB+Kanamycin, and incubate with shaking at 37° C. until OD=0.5-0.6.
6) Move to 16° C. shaker for 15 minutes.
   a) Induce protein expression.
      i) Add 200 mM IPTG
      ii) Shake at 16° C. overnight
7) Purify protein.
   i) In morning, pellet cells by centrifugation.
   ii) Resuspend in 30 mL Lysis Buffer (20 mM Tris pH 8.0, 400 mM NaCl, 1% Triton) in a fresh 50 mL conical tube.
   iii) Add 10 μL DNase I (Thermo Cat EN0525) and 10 μL Benzonase (Sigma Cat #E1014).
   iv) Add 2 mM MgCl₂.
   v) Sonicate sample.
   vi) Spin sample at 12,000×g for 15 min.
   vii) Transfer supernatant to a fresh tube.
   viii) Add binding agents.
      (1) Ni-NTA Agarose Resin (Qiagen Cat #30230)
      (2) Imidazol to 15 mM
      (3) DTT to 2 mM
   ix) Rotate at 4° C. for 2-3 hours.
   x) Transfer beads into an empty gravity flow column.
   xi) Wash with 60 mL TBS Wash Buffer (20 mM Tris pH 8.0, 400 mM NaCl, 20 mM Imidazole pH 8.0).
   xii) Elute with TBS Elution buffer (20 mM Tris pH 8.0, 400 mM NaCl, 250 mM Imidazole pH 8.0) in 8×1 mL fractions.
   xiii) Use NanoDrop to calculate A280 of each fraction; store fractions at 4° C.
   xiv) Analyze each fraction by SDS-PAGE and pool fractions containing protein of interest using Bio-Rad 10DG Desalting Columns.
   xv) Concentrate to approximately 100 μM by gravity centrifugation concentrators. Store at 4° C.
8) Assay prep: Follow assay set up instructions from EnzChek™ Phosphate Assay Kit for reagent prep.
   a) Set up reactions in 384 well format using a flat, clear plate.
   b) Set up reactions using a volume between 15-50 μL.
   c) Generate reaction buffer of 50 mM Tris·HCl (pH 7.5), 50 mM NaCl, 1 mM MgCl2, 0.1 mM sodium azide, 200 μM MESG, and 1 U PNP.
   d) Add 2 mM Morc3 protein as experimental enzyme or equivalent volume of HBS buffer as control.
   e) Add drug.
   f) Measure absorbance at 360 for 10 minutes using a plate reader such as a Tecan Spark.
   g) Add experimental substrates: 2 mM ATP, 1 μM 601 DNA (EpiCypher Cat #18-0005 Nucleosome Assembly 601 Sequence DNA, Biotinylated), and 50 μM H3K4me3 peptide in reactions containing CW domain (Anaspec Cat #AS-64371-1).
   h) Measure absorbance at 360 for 1 hour using a plate reader such as a Tecan Spark.
9) Analyze data.
   a) Find reading value by subtracting absorbance reading of buffer control from absorbance reading of protein sample.

### Statistical Analysis

Data was analyzed for statistical significant differences using GraphPad Prism 8. Gene expression values were log 2 transformed and viral titers were log 10 transformed for statistical analysis. Statistical tests are indicated in the Figure legends and were RM one-way or two-way ANOVA with Geisser-Greenhouse correction and Dunnett's post hoc test or paired, two-sided T-test. In case of two-way ANOVA the factors were genotype and stimulus. Normality of data was assumed and not tested. * p<0.05; ** p<0.01 *** p<0.001.

### Data Availability

RNA-seq datasets were deposited at GEO.

### REFERENCES

The following references are herein incorporated by reference in their entirety with the exception that, should the scope and meaning of a term conflict with a definition explicitly set forth herein, the definition explicitly set forth herein controls:
Janeway, C. A., Jr. Approaching the asymptote? Evolution and revolution in immunology. Cold Spring Harb Symp Quant Biol 54 Pt 1, 1-13, doi:10.1101/sqb.1989.054.01.003 (1989).
Lopes Fischer, N., Naseer, N., Shin, S. & Brodsky, I. E. Effector-triggered immunity and pathogen sensing in metazoans. Nat Microbiol 5, 14-26, doi:10.1038/s41564-019-0623-2 (2020).
Van der Biezen, E. A. & Jones, J. D. Plant disease-resistance proteins and the gene-for-gene concept. Trends Biochem Sci 23, 454-456, doi:10.1016/s0968-0004(98)01311-5 (1998).
Jones, J. D. & Dangl, J. L. The plant immune system. Nature 444, 323-329, doi:10.1038/nature05286 (2006).
Sloan, E. et al. Analysis of the SUMO2 Proteome during HSV-1 Infection. PLoS Pathog 11, e1005059, doi:10.1371/journal.ppat.1005059 (2015).
Sloan, E., Orr, A. & Everett, R. D. MORC3, a Component of PML Nuclear Bodies, Has a Role in Restricting Herpes Simplex Virus 1 and Human Cytomegalovirus. J Virol 90, 8621-8633, doi:10.1128/JVI.00621-16 (2016).
Van de Weyer, A. L. et al. A Species-Wide Inventory of NLR Genes and Alleles in Arabidopsis thaliana. Cell 178, 1260-1272 e1214, doi:10.1016/j.cell.2019.07.038 (2019).
Boyden, E. D. & Dietrich, W. F. Nalplb controls mouse macrophage susceptibility to anthrax lethal toxin. Nat Genet 38, 240-244, doi:10.1038/ng1724 (2006).
Keestra, A. M. et al. Manipulation of small Rho GTPases is a pathogen-induced process detected by NOD1. Nature 496, 233-237, doi:10.1038/nature12025 (2013).
Xu, H. et al. Innate immune sensing of bacterial modifications of Rho GTPases by the Pyrin inflammasome. Nature 513, 237-241, doi:10.1038/nature13449 (2014).
Luecke, S. & Paludan, S. R. Innate recognition of alphaherpesvirus DNA. Adv Virus Res 92, 63-100, doi:10.1016/bs.aivir.2014.11.003 (2015).
Hopfner, K. P. & Hornung, V. Molecular mechanisms and cellular functions of cGAS-STING signalling. Nat Rev Mol Cell Biol 21, 501-521, doi:10.1038/s41580-020-0244-x (2020).
Ishikawa, H., Ma, Z. & Barber, G. N. STING regulates intracellular DNA-mediated, type I interferon-dependent innate immunity. Nature 461, 788-792, doi:10.1038/nature08476 (2009).
Gaidt, M. M. et al. The DNA Inflammasome in Human Myeloid Cells Is Initiated by a STING-Cell Death Program Upstream of NLRP3. Cell 171, 1110-1124 e1118, doi:10.1016/j.cell.2017.09.039 (2017).
Kobiler, O. & Weitzman, M. D. Herpes simplex virus replication compartments: From naked release to recombining together. PLoS Pathog 15, e1007714, doi:10.1371/journal.ppat.1007714 (2019).
Charman, M. & Weitzman, M. D. Replication Compartments of DNA Viruses in the Nucleus: Location, Location, Location. Viruses 12, doi:10.3390/v12020151 (2020).
Scherer, M. & Stamminger, T. Emerging Role of PML Nuclear Bodies in Innate Immune Signaling. J Virol 90, 5850-5854, doi:10.1128/JVI.01979-15 (2016).
Everett, R. D., Boutell, C. & Hale, B. G. Interplay between viruses and host sumoylation pathways. Nat Rev Microbiol 11, 400-411, doi:10.1038/nrmicro3015 (2013).
Roizman, B. & Whitley, R. J. An inquiry into the molecular basis of HSV latency and reactivation. Annu Rev Microbiol 67, 355-374, doi:10.1146/annurev-micro-092412-155654 (2013).
Everett, R. D. et al. PML contributes to a cellular mechanism of repression of herpes simplex virus type 1 infection that is inactivated by ICP0. J Virol 80, 7995-8005, doi:10.1128/JVI.00734-06 (2006).
Everett, R. D., Parada, C., Gripon, P., Sirma, H. & Orr, A. Replication of ICP0-null mutant herpes simplex virus type 1 is restricted by both PML and Sp100. J Virol 82, 2661-2672, doi:10.1128/JVI.02308-07 (2008).
Merkl, P. E., Orzalli, M. H. & Knipe, D. M. Mechanisms of Host IFI16, PML, and Daxx Protein Restriction of Herpes Simplex Virus 1 Replication. J Virol 92, doi:10.1128/JVI.00057-18 (2018).
McFarlane, S. et al. The histone chaperone HIRA promotes the induction of host innate immune defenses in response to HSV-1 infection. PLoS Pathog 15, e1007667, doi:10.1371/journal.ppat.1007667 (2019).
Gaidt, M. M. et al. Human Monocytes Engage an Alternative Inflammasome Pathway. Immunity 44, 833-846, doi:10.1016/j.immuni.2016.01.012 (2016).
Rapino, F. et al. C/EBPalpha induces highly efficient macrophage transdifferentiation of B lymphoma and leukemia cell lines and impairs their tumorigenicity. Cell Rep 3, 1153-1163, doi:10.1016/j.celrep.2013.03.003 (2013).
Fitzgerald, K. A. et al. IKKepsilon and TBK1 are essential components of the IRF3 signaling pathway. Nat Immunol 4, 491-496, doi:10.1038/ni921 (2003).
Sato, M. et al. Distinct and essential roles of transcription factors IRF-3 and IRF-7 in response to viruses for IFN-alpha/beta gene induction. Immunity 13, 539-548, doi:10.1016/s1074-7613(00)00053-4 (2000).
Burleigh, K. et al. Human DNA-PK activates a STING-independent DNA sensing pathway. Sci Immunol 5, doi:10.1126/sciimmunol.aba4219 (2020).
Rodriguez, M. C., Dybas, J. M., Hughes, J., Weitzman, M. D. & Boutell, C. The HSV-1 ubiquitin ligase ICP0: Modifying the cellular proteome to promote infection. Virus Res 285, 198015, doi:10.1016/j.virusres.2020.198015 (2020).
Boutell, C. & Everett, R. D. Regulation of alphaherpesvirus infections by the ICP0 family of proteins. J Gen Virol 94, 465-481, doi:10.1099/vir.0.048900-0 (2013).
Ullman, A. J. & Hearing, P. Cellular proteins PML and Daxx mediate an innate antiviral defense antagonized by the adenovirus E4 ORF3 protein. J Virol 82, 7325-7335, doi:10.1128/JVI.00723-08 (2008).
Ou, H. D. et al. A structural basis for the assembly and functions of a viral polymer that inactivates multiple tumor suppressors. Cell 151, 304-319, doi:10.1016/j.cell.2012.08.035 (2012).
Crowl, J. T. & Stetson, D. B. SUMO2 and SUMO3 redundantly prevent a noncanonical type I interferon response. Proc Natl Acad Sci USA 115, 6798-6803, doi:10.1073/pnas.1802114115 (2018).
Ferri, F. et al. TRIM33 switches off Ifnb1 gene transcription during the late phase of macrophage activation. Nat Commun 6, 8900, doi:10.1038/ncomms9900 (2015).
Malakhova, O. A. et al. UBP43 is a novel regulator of interferon signaling independent of its ISG15 isopeptidase activity. EMBO J 25, 2358-2367, doi:10.1038/sj.emboj.7601149 (2006).
Li, D. Q., Nair, S. S. & Kumar, R. The MORC family: new epigenetic regulators of transcription and DNA damage response. Epigenetics 8, 685-693, doi:10.4161/epi.24976 (2013).
Li, S. et al. Mouse MORC3 is a GHKL ATPase that localizes to H3K4me3 marked chromatin. Proc Natl Acad Sci USA 113, E5108-5116, doi:10.1073/pnas.1609709113 (2016).
Rosendorff, A. et al. NXP-2 association with SUMO-2 depends on lysines required for transcriptional repression. Proc Natl Acad Sci USA 103, 5308-5313, doi:10.1073/pnas.0601066103 (2006).
Mimura, Y., Takahashi, K., Kawata, K., Akazawa, T. & Inoue, N. Two-step colocalization of MORC3 with PML nuclear bodies. J Cell Sci 123, 2014-2024, doi:10.1242/jcs.063586 (2010).
Takahashi, K. et al. Dynamic regulation of p53 subnuclear localization and senescence by MORC3. Mol Biol Cell 18, 1701-1709, doi:10.1091/mbc.e06-08-0747 (2007).
Jadhav, G., Teguh, D., Kenny, J., Tickner, J. & Xu, J. Morc3 mutant mice exhibit reduced cortical area and thickness, accompanied by altered haematopoietic stem cells niche and bone cell differentiation. Sci Rep 6, 25964, doi:10.1038/srep25964 (2016).
Ver, L. S., Marcos-Villar, L., Landeras-Bueno, S., Nieto, A. & Ortin, J. The Cellular Factor NXP2/MORC3 Is a Positive Regulator of Influenza Virus Multiplication. J Virol 89, 10023-10030, doi:10.1128/JVI.01530-15 (2015).
Zhang, Y. et al. MORC3 Is a Target of the Influenza A Viral Protein NS1. Structure 27, 1029-1033 e1023, doi:10.1016/j.str.2019.03.015 (2019).
Groh, S. et al. Morc3 silences endogenous retroviruses by enabling Daxx-mediated H3.3 incorporation. bioRxiv, 2020.2011.2012.380204, doi:10.1101/2020.11.12.380204 (2020).
Steinberg, C. et al. The IFN regulatory factor 7-dependent type I IFN response is not essential for early resistance against murine cytomegalovirus infection. Eur J Immunol 39, 1007-1018, doi:10.1002/eji.200838814 (2009).
Decque, A. et al. Sumoylation coordinates the repression of inflammatory and anti-viral gene-expression programs during innate sensing. Nat Immunol 17, 140-149, doi:10.1038/ni.3342 (2016).
Lorkovic, Z. J., Naumann, U., Matzke, A. J. & Matzke, M. Involvement of a GHKL ATPase in RNA-directed DNA methylation in Arabidopsis thaliana. Curr Biol 22, 933-938, doi:10.1016/j.cub.2012.03.061 (2012).
Tchasovnikarova, I. A. et al. Hyperactivation of HUSH complex function by Charcot-Marie-Tooth disease mutation in MORC2. Nat Genet 49, 1035-1044, doi:10.1038/ng.3878 (2017).
Kim, H. et al. The Gene-Silencing Protein MORC-1 Topologically Entraps DNA and Forms Multimeric Assemblies to Cause DNA Compaction. Mol Cell 75, 700-710 e706, doi:10.1016/j.molcel.2019.07.032 (2019).
Ladle, R. J. Parasites and sex: Catching the red queen. Trends Ecol Evol 7, 405-408, doi:10.1016/0169-5347(92)90021-3 (1992).
Daugherty, M. D. & Malik, H. S. Rules of engagement: molecular insights from host-virus arms races. Annu Rev Genet 46, 677-700, doi:10.1146/annurev-genet-110711-155522 (2012).
Livingston, J. H. & Crow, Y. J. Neurologic Phenotypes Associated with Mutations in TREX1, RNASEH2A, RNASEH2B, RNASEH2C, SAMHD1, ADAR1, and IFIH1: Aicardi-Goutieres Syndrome and Beyond. Neuropediatrics 47, 355-360, doi:10.1055/s-0036-1592307 (2016).
Zhang, X. et al. Human intracellular ISG15 prevents interferon-alpha/beta over-amplification and auto-inflammation. Nature 517, 89-93, doi:10.1038/nature13801 (2015).
Dzimianski, J. V., Scholte, F. E. M., Bergeron, E. & Pegan, S. D. ISG15: It's Complicated. J Mol Biol 431, 4203-4216, doi:10.1016/j.jmb.2019.03.013 (2019).
Raulet, D. H. & Vance, R. E. Self-tolerance of natural killer cells. Nat Rev Immunol 6, 520-531, doi:10.1038/nri1863 (2006).
Dondelinger, Y., Darding, M., Bertrand, M. J. & Walczak, H. Poly-ubiquitination in TNFR1-mediated necroptosis. Cell Mol Life Sci 73, 2165-2176, doi:10.1007/s00018-016-2191-4 (2016).
Greene, T. T. et al. A Herpesviral induction of RAE-1 NKG2D ligand expression occurs through release of HDAC mediated repression. Elife 5, doi:10.7554/eLife.14749 (2016).
Peterson, L. W. et al. RIPK1-dependent apoptosis bypasses pathogen blockade of innate signaling to promote immune defense. J Exp Med 214, 3171-3182, doi:10.1084/jem.20170347 (2017).
Blaho, J. A., Morton, E. R. & Yedowitz, J. C. Herpes simplex virus: propagation, quantification, and storage. Curr Protoc Microbiol Chapter 14, Unit 14E 11, doi:10.1002/9780471729259.mc14e01s00 (2005).
Labun, K. et al. CHOPCHOP v3: expanding the CRISPR web toolbox beyond genome editing. Nucleic Acids Res 47, W171-W174, doi:10.1093/nar/gkz365 (2019).
Schmidt, T., Schmid-Burgk, J. L. & Hornung, V. Synthesis of an arrayed sgRNA library targeting the human genome. Sci Rep 5, 14987, doi:10.1038/srep14987 (2015).
Schmidt, T., Schmid-Burgk, J. L., Ebert, T. S., Gaidt, M. M. & Hornung, V. Designer Nuclease-Mediated Generation of Knockout THP1 Cells. Methods Mol Biol 1338, 261-272, doi:10.1007/978-1-4939-2932-0_19 (2016).
Sanjana, N. E., Shalem, 0. & Zhang, F. Improved vectors and genome-wide libraries for CRISPR screening. Nat Methods 11, 783-784, doi:10.1038/nmeth.3047 (2014).
Bartok, E. et al. iGLuc: a luciferase-based inflammasome and protease activity reporter. Nat Methods 10, 147-154, doi:10.1038/nmeth.2327 (2013).
Spahn, P. N. et al. PinAPL-Py: A comprehensive web-application for the analysis of CRISPR/Cas9 screens. Sci Rep 7, 15854, doi:10.1038/s41598-017-16193-9 (2017).
Andrews, S. FastQC: A Quality Control Tool for High Throughput Sequence Data. (2010).
Langmead, B. & Salzberg, S. L. Fast gapped-read alignment with Bowtie 2. Nat Methods 9, 357-359, doi:10.1038/nmeth.1923 (2012).
Li, B. & Dewey, C. N. RSEM: accurate transcript quantification from RNA-Seq data with or without a reference genome. BMC Bioinformatics 12, 323, doi:10.1186/1471-2105-12-323 (2011).
Love, M. I., Huber, W. & Anders, S. Moderated estimation of fold change and dispersion for RNA-seq data with DESeq2. Genome Biol 15, 550, doi:10.1186/s13059-014-0550-8 (2014).
Yu, G., Wang, L. G., Han, Y. & He, Q. Y. clusterProfiler: an R package for comparing biological themes among gene clusters. OMICS 16, 284-287, doi:10.1089/omi.2011.0118 (2012).
Liberzon, A. et al. Molecular signatures database (MSigDB) 3.0. Bioinformatics 27, 1739-1740, doi:10.1093/bioinformatics/btr260 (2011).
Dobin, A. et al. STAR: ultrafast universal RNA-seq aligner. Bioinformatics 29, 15-21, doi:10.1093/bioinformatics/bts635 (2013).
Criscione, S. W., Zhang, Y., Thompson, W., Sedivy, J. M. & Neretti, N. Transcriptional landscape of repetitive elements in normal and cancer human cells. BMC Genomics 15, 583, doi:10.1186/1471-2164-15-583 (2014).
nerettilab. RepEnrich2. https://github.com/nerettilab/RepEnrich2 (2019).
Robinson, M. D., McCarthy, D. J. & Smyth, G. K. edgeR: a Bioconductor package for differential expression analysis of digital gene expression data. Bioinformatics 26, 139-140, doi:10.1093/bioinformatics/btp616 (2010).
Zhang Y, Klein B J, Cox K L, et al. Mechanism for autoinhibition and activation of the MORC3 ATPase. PNAS USA 2019; 116(13):6111-6119. doi:10.1073/pnas.1819524116

All scientific and technical terms used in this application have meanings commonly used in the art unless otherwise specified.

As used herein, a human MORC3 comprises at least 90% sequence identity to Accession No. NP_001307374 or NP_056173.1. In some embodiments, a human MORC3 comprises at least 95% sequence identity to Accession No. NP_001307374 or NP_056173.1. In some embodiments, a human MORC3 comprises at least 97% sequence identity to Accession No. NP_001307374 or NP_056173.1. In some embodiments, the sequence of a human MORC3 is NP_001307374.1 or NP_056173.1. In some embodiments, a “MORC3 protein” refers to a protein having at least 90% sequence identity to SEQ ID NO: 1. In some embodiments, an “ICP0 protein” refers to a protein having at least about 95% sequence identity to SEQ ID NO: 2.

As used herein, the terms “subject”, “patient”, and “individual” are used interchangeably to refer to humans and non-human animals. The terms “non-human animal” and “animal” refer to all non-human vertebrates, e.g., non-human mammals and non-mammals, such as non-human primates, horses, sheep, dogs, cows, pigs, chickens, and other veterinary subjects and test animals. In some embodiments, the subject is a mammal. In some embodiments, the subject is a human.

As used herein, the term “diagnosing” refers to the physical and active step of informing, i.e., communicating verbally or by writing (on, e.g., paper or electronic media), another party, e.g., a patient, of the diagnosis. Similarly, “providing a prognosis” refers to the physical and active step of informing, i.e., communicating verbally or by writing (on, e.g., paper or electronic media), another party, e.g., a patient, of the prognosis.

The use of the singular can include the plural unless specifically stated otherwise. As used in the specification and the appended claims, the singular forms “a”, “an”, and “the” can include plural referents unless the context clearly dictates otherwise.

As used herein, “and/or” means “and” or “or”. For example, “A and/or B” means “A, B, or both A and B” and “A, B, C, and/or D” means “A, B, C, D, or a combination thereof” and said “A, B, C, D, or a combination thereof” means any subset of A, B, C, and D, for example, a single member subset (e.g., A or B or C or D), a two-member subset (e.g., A and B; A and C; etc.), or a three-member subset (e.g., A, B, and C; or A, B, and D; etc.), or all four members (e.g., A, B, C, and D).

As used herein, the phrase “one or more of”, e.g., “one or more of A, B, and/or C” means “one or more of A”, “one or more of B”, “one or more of C”, “one or more of A and one or more of B”, “one or more of B and one or more of C”, “one or more of A and one or more of C” and “one or more of A, one or more of B, and one or more of C”.

As used herein, the phrase “consists essentially of” in the context of a given ingredient in a composition, means that the composition may include additional ingredients so long as the additional ingredients do not adversely impact the activity, e.g., biological or pharmaceutical function, of the given ingredient.

The phrase “comprises, consists essentially of, or consists of A” is used as a tool to avoid excess page and translation fees and means that in some embodiments the given thing at issue: comprises A, consists essentially of A, or consists of A. For example, the sentence “In some embodiments, the composition comprises, consists essentially of, or consists of A” is to be interpreted as if written as the following three separate sentences: “In some embodiments, the composition comprises A. In some embodiments, the composition consists essentially of A. In some embodiments, the composition consists of A.”

Similarly, a sentence reciting a string of alternates is to be interpreted as if a string of sentences were provided such that each given alternate was provided in a sentence by itself. For example, the sentence “In some embodiments, the composition comprises A, B, or C” is to be interpreted as if written as the following three separate sentences: “In some embodiments, the composition comprises A. In some embodiments, the composition comprises B. In some embodiments, the composition comprises C.” As another example, the sentence “In some embodiments, the composition comprises at least A, B, or C” is to be interpreted as if written as the following three separate sentences: “In some embodiments, the composition comprises at least A. In some embodiments, the composition comprises at least B. In some embodiments, the composition comprises at least C.”

As used herein, the terms “protein”, “polypeptide” and “peptide” are used interchangeably to refer to two or more amino acids linked together. Groups or strings of amino acid abbreviations are used to represent peptides. Except when specifically indicated, peptides are indicated with the N-terminus on the left and the sequence is written from the N-terminus to the C-terminus. Except when specifically indicated, peptides are indicated with the N-terminus on the left and the sequences are written from the N-terminus to the C-terminus. Similarly, except when specifically indicated, nucleic acid sequences are indicated with the 5′ end on the left and the sequences are written from 5′ to 3′.

As used herein, a given percentage of “sequence identity” refers to the percentage of nucleotides or amino acid residues that are the same between sequences, when compared and optimally aligned for maximum correspondence over a given comparison window, as measured by visual inspection or by a sequence comparison algorithm in the art, such as the BLAST algorithm, which is described in Altschul et al., (1990) J Mol Biol 215:403-410. Software for performing BLAST (e.g., BLASTP and BLASTN) analyses is publicly available through the National Center for Biotechnology Information (ncbi.nlm.nih.gov). The comparison window can exist over a given portion, e.g., a functional domain, or an arbitrarily selection a given number of contiguous nucleotides or amino acid residues of one or both sequences. Alternatively, the comparison window can exist over the full length of the sequences being compared. For purposes herein, where a given comparison window (e.g., over 80% of the given sequence) is not provided, the recited sequence identity is over 100% of the given sequence. Additionally, for the percentages of sequence identity of the proteins provided herein, the percentages are determined using BLASTP 2.8.0+, scoring matrix BLOSUM62, and the default parameters available at blast.ncbi.nlm.nih.gov/Blast.cgi. See also Altschul, et al., (1997) Nucleic Acids Res 25:3389-3402; and Altschul, et al., (2005) FEBS J 272:5101-5109.

Optimal alignment of sequences for comparison can be conducted, e.g., by the local homology algorithm of Smith & Waterman, Adv Appl Math 2:482 (1981), by the homology alignment algorithm of Needleman & Wunsch, J Mol Biol 48:443 (1970), by the search for similarity method of Pearson & Lipman, PNAS USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection.

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

1. A method of increasing or decreasing the amount of endogenous interferon in a subject or treating an interferon disorder in the subject, which comprises administering to the subject a MORC3 therapeutic agent.

2. (canceled)

3. The method according claim 1, which comprises decreasing the amount of endogenous interferon in the subject by administering the MORC3 therapeutic agent, wherein the MORC3 therapeutic agent is a MORC3 activator.

4. The method according claim 1, which comprises decreasing the amount of endogenous interferon in the subject by administering the MORC3 therapeutic agent, which is a MORC3 protein having at least about 90% sequence identity to SEQ ID NO: 1, and/or stabilizes the expression of MORC3.

5. The method according claim 1, which comprises increasing the amount of endogenous interferon in the subject by administering the MORC3 therapeutic agent, wherein the MORC3 therapeutic agent is a MORC3 inhibitor.

6. The method according claim 1, which comprises increasing the amount of endogenous interferon in the subject by administering the MORC3 therapeutic agent, which is an siRNA, an ATPase inhibitor (e.g., a small molecule ATPase inhibitor), and/or an ICP0 protein having at least about 95% sequence identity to SEQ ID NO: 2.

7. The method according to claim 1, wherein the subject is in need thereof.

8. (canceled)

9. The method according to claim 1, wherein the interferon disorder is an IFNB1 Disorder, an IFN Excess Disorder, or an IFN Deficiency Disorder.

10. (canceled)

11. The method according to claim 1, wherein the interferon disorder is an autoimmune or inflammatory disease.

12. The method according to claim 1, wherein the interferon disorder is rheumatoid arthritis, psoriasis, vitiligo, hypothyroidism, hyperthyroidism, idiopathic thrombocytopenic purpura, autoimmune hemolytic anemia, myasthenia gravis, Addison disease, celiac disease, polymyositis, superimposed autoimmune hepatitis, or multiple sclerosis.

13-16. (canceled)

17. The method according to claim 8, wherein the IFN Deficiency Disorder is a cancer or a viral infection.

18. The method according to claim 17, wherein the cancer is a leukemia, a lymphoma, a melanoma, a sarcoma, or an adenocarcinoma.

19. The method according to claim 17, wherein the cancer is colon cancer.

20. (canceled)

21. The method according to claim 17, wherein the viral infection is caused by a herpes virus, a hepatitis virus, or a coronavirus.

22. (canceled)

23. An assay method for determining whether a candidate compound is a MORC3 inhibitor, which comprises contacting the candidate compound with a monocyte and measuring any interferon response induced thereby in the monocyte.

24. The assay method according to claim 23, and further comprising contacting the candidate compound with a genetically modified monocyte that deficient in MORC3 activity, measuring any interferon response induced thereby in the genetically modified monocyte, and comparing the interferon response in the monocyte to the interferon response in the genetically modified cell.

25. A method of modulating interferon expression by a cell, which comprises
1) increasing interferon expression by the cell by (a) reducing the amount of a MORC3 protein in the cell, (b) increasing MRE activity in the cell, or both (a) and (b); or
2) decreasing interferon expression by the cell by (a) increasing the amount of a MORC3 protein in the cell, (b) reducing MRE activity in the cell, or both (a) and (b).

26. (canceled)


SEQUENCE LISTING (62 sequences)

SEQ ID NO: 1 - protein, 939 aa, Artificial Sequence
  MORC3 protein
MAAQPPRGIRLSALCPKFLHTNSTSHTWPFSAVAELIDNAYDPDVNAKQIWIDKTVINDHICLTFTDNGNGMTSDKLHKMLSFGFSDKVTMNGHVPVGLYGNGFKSGSMRLGKDAIVFTKNGESMSVGLLSQTYLEVIKAEHVVVPIVAFNKHRQMINLAESKASLAAILEHSLFSTEQKLLAELDAIIGKKGTRIIIWNLRSYKNATEFDFEKDKYDIRIPEDLDEITGKKGYKKQERMDQIAPESDYSLRAYCSILYLKPRMQIILRGQKVKTQLVSKSLAYIERDVYRPKFLSKTVRITFGFNCRNKDHYGIMMYHRNRLIKAYEKVGCQLRANNMGVGVVGIIECNFLKPTHNKQDFDYTNEYRLTITALGEKLNDYWNEMKVKKNTEYPLNLPVEDIQKRPDQTWVQCDACLKWRKLPDGMDQLPEKWYCSNNPDPQFRNCEVPEEPEDEDLVHPTYEKTYKKTNKEKFRIRQPEMIPRINAELLFRPTALSTPSFSSPKESVPRRHLSEGTNSYATRLLNNHQVPPQSEPESNSLKRRLSTRSSILNAKNRRLSSQFENSVYKGDDDDEDVIILEENSTPKPAVDHDIDMKSEQSHVEQGGVQVEFVGDSEPCGQTGSTSTSSSRCDQGNTAATQTEVPSLVVKKEETVEDEIDVRNDAVILPSCVEAEAKIHETQETTDKSADDAGCQLQELRNQLLLVTEEKENYKRQCHMFTDQIKVLQQRILEMNDKYVKKETCHQSTETDAVFLLESINGKSESPDHMVSQYQQALEEIERLKKQCSALQHVKAECSQCSNNESKSEMDEMAVQLDDVFRQLDKCSIERDQYKSEVELLEMEKSQIRSQCEELKTEVEQLKSTNQQTATDVSTSSNIEESVNHMDGESLKLRSLRVNVGQLLAMIVPDLDLQQVNYDVDVVDEILGQVVEQMSEISST

SEQ ID NO: 2 - protein, 775 aa, Artificial Sequence
  ICP0 protein
MEPRPGASTRRPEGRPQREPAPDVWVFPCDRDLPDSSDSEAETEVGGRGDADHHDDDSASEADSTDTELFETGLLGPQGVDGGAVSGGSPPREEDPGSCGGAPPREDGGSDEGDVCAVCTDEIAPHLRCDTFPCMHRFCIPCMKTWMQLRNTCPLCNAKLVYLIVGVTPSGSFSTIPIVNDPQTRMEAEEAVRAGTAVDFIWTGNQRFAPRYLTLGGHTVRALSPTHPEPTTDEDDDDLDDADYVPPAPRRTPRAPPRRGAAAPPVTGGASHAAPQPAAARTAPPSAPIGPHGSSNTNTTTNSSGGGGSRQSRAAAPRGASGPSGGVGVGVGVVEAEAGRPRGRTGPLVNRPAPLANNRDPIVISDSPPASPHRPPAAPMPGSAPRPGPPASAAASGPARPRAAVAPCVRAPPPGPGPRAPAPGAEPAARPADARRVPQSHSSLAQAANQEQSLCRARATVARGSGGPGVEGGHGPSRGAAPSGAAPLPSAASVEQEAAVRPRKRRGSGQENPSPQSTRPPLAPAGAKRAATHPPSDSGPGGRGQGGPGTPLTSSAASASSSSASSSSAPTPAGAASSAAGAASSSASASSGGAVGALGGRQEETSLGPRAASGPRGPRKCARKTRHAETSGAVPAGGLTRYLPISGVSSVVALSPYVNKTITGDCLPILDMETGNIGAYVVLVDQTGNMATRLRAAVPGWSRRTLLPETAGNHVMPPEYPTAPASEWNSLWMTPVGNMLFDQGTLVGALDFRSLRSRHPWSGEQGASTRDEGKQ

SEQ ID NO: 3 - DNA, 3200 bp, Artificial Sequence
  MRE sequence
cctaccaagtgtacattagggttccattatttgcatatcctgggcaacactttttctggtgtttttgttgctattgtttttatagcaacaaataggttctaataggtataggctgatatctcattgtggttttgatttggtttggtttttttccttctgattagtgatattggtcatcttcatatgcttgttggacatttgtatatcatctttgggaaaatgtctaagttctttgcccatttttatatgctctctttttttctatgttactccttttttagctgctcttttttccttttgttgattctccctctgccctctctttttcagtttctagttgttctgcttttcctgctaattctgcatgtgcttctcttttctcctatgcttctttctgctgtttttcatttttctttctctgcctgctttttcagtatctattcattttgcttctgtttctctttttcaacttctgttaattctgtttctttttctgctgattccacatagcttctcctttttgcagctattttccttctgttgactctgccctatttcttcttccaccttttcagtgtgctgattttacctccgcttctccttgttcccatgcttctcctttctgcagctattttaccttatgctgactttcctttattctggttccctcttctttcagcatctgctgatgtggccactgctccttgttcctgtgcttctgctttctgcagctgatttacttctgctgactttgccctgttctggttccctcttctttcagcatctgctgatgtggcctctgcttctccttgttcctgtgcttcttctttctgcagctattttaccttctgctgactttgccttgttctgattccctctttcagtatcatctgatgtggcctctgcttctccttgttcctgtgcttctcctttctgcagctatttaaccttctgctgactttgccttattctggttccctcttctttcagcatctgctgatgtggcctctgcttctctttgttcctgtgcttctcctttctgcagctgatttacttctgctgactttgccctgttctgcttccgtcttctttcagcatctgctgatgtgggctctgcttctcctttttcccatgtttctcctttctgcagcttttttttttttttctgctgactcttgcttccccacttttagcatctgctaatttggtctctgcttctcctttttcctatgcttctcattcctgctgctgttttcacttttgctgactctgccctacttcccccttcttttagcatctgttcatggcctctgcttctcctttttcctatgtttctcctttctgcagctgtttttttcctgctgactcttgcttccccattttagcatctgctaatttggcctctgcttctcctttttcctatgcttctcatttctgctgctgttttcacgtttgctgactctgccctacttccctcttctttcagcatctgttcatggcctctgcttctcctttttcctatgtttctcctttctgcagctgattttttcctgctgactcttgcttccccattttagcatctgctaatttggcctctgcttctcctttttcctatgcttctcatttctgctgctgttttcatgtttgctgactctgccctacttccccctactttcagcatctgttcatggcctctgcttctcctttttcctatgtttctcctttctgtagctgtttttttcctgctgactcttgcttccccattttagcatctgctaatttggcctctgcttctcctttttcctgtgcttctcttttctgctgctgttttcacttttgctgtctctgccctacttcccccttctttcagcatctgttcatggcctctgcttctcctttttcctatgtttctcctttctgtagctgtttttttcctgctgactcttgcttccccattttagcatctgctaatttggtctctgcttctcctttttcctgtgcttctcttttctgctgctgttttcacttttgctgtctctgccctacttcccccttctttcagcatctgttcatggcctctgcttctcctttgtcctatgtttgtcctttctgtagctgtttttttcctgctgactcttgcttccccattttagcatctgctaatttggcctctgcttctccttttccctgtgcttctcttttctgctgctgttttcacttttgctgtctctgccctacttcccccttctttcagcatctgttcatggcctctgcttctcctttttcctatgtttgtcctttctgtagctgtttttttcctgctgactcttgcttccccattttagcatctgctaatttggcctctgcttctcctttttcctatgcttctcatttctgctgctgttttcacgtttgctgactctaccctacttcccccttctttcagcatctgttcatggcctctgcttctcctttttcctatgtttctcctttctgtagctgtttttttcctgctgactcttgcttccccattttagcatctgctaatttggcctctgcttctcctttttcctgtgcttctcatttctgctgctgttttcacgtttgctgactctggctccacatctgcttctcccatctttcagcatctactgattctgctactttttctgatgattttgcctctgcttctccttcttcctatgcttctcctttctgaagctgtttttccttttgctgactctacttccacgctgattccccctttttcagcatcttctgattcaggtgcttttttgctgatcctgcctctgcttttccttttcctacctctacttttccttttgttgattctgcttctctttctgctgatgctaatttcctttctactgccgatgttgatcctgctccttttgcttctccttggtcttctacattttaatgtgttatactatttctggcctcctcgtagtttcacgtttacatggcccttaatggctattctattcctggttgcaccatatgtttgcactcacaaatgctctactatctgtttttcaatttctaattaccctatacacacttcctttaaataggaatataatattaaagtaatggtactatgtcataggcagctggttaactgatggactagctgttacacagcccttagatgctcacttctagaacggtcaccatgg

SEQ ID NO: 4 - DNA, 20 bp, Artificial Sequence
  RSAD2 forward primer
caactacaaatgcggcttct

SEQ ID NO: 5 - DNA, 21 bp, Artificial Sequence
  RSAD2 reverse primer
atcttctccataccagcttcc

SEQ ID NO: 6 - DNA, 20 bp, Artificial Sequence
  CXCL10 forward primer
tctgaatccagaatcgaagg

SEQ ID NO: 7 - DNA, 20 bp, Artificial Sequence
  CXCL10 reverse primer
ctctgtgtggtccatccttg

SEQ ID NO: 8 - DNA, 20 bp, Artificial Sequence
  GAPDH forward primer
gagtcaacggatttggtcgt

SEQ ID NO: 9 - DNA, 20 bp, Artificial Sequence
  GAPDH reverse primer
gacaagcttcccgttctcag

SEQ ID NO: 10 - DNA, 20 bp, Artificial Sequence
  IFNB1 forward primer
cagcatctgctggttgaaga

SEQ ID NO: 11 - DNA, 20 bp, Artificial Sequence
  IFNB1 reverse primer
cattacctgaaggccaagga

SEQ ID NO: 12 - DNA, 20 bp, Artificial Sequence
  MLLT3 forward primer
gagcacagtaacatacagca

SEQ ID NO: 13 - DNA, 20 bp, Artificial Sequence
  MLLT3 reverse primer
ggcaaaatgaaaccagcata

SEQ ID NO: 14 - DNA, 23 bp, Artificial Sequence
  sgRNA
gatgaactttgacatccctgagg

SEQ ID NO: 15 - DNA, 23 bp, Artificial Sequence
  STING sgRNA target site
gcgggccgaccgcatttgggagg

SEQ ID NO: 16 - DNA, 23 bp, Artificial Sequence
  TBK1 sgRNA target site
acagtgtataaactcccacatgg

SEQ ID NO: 17 - DNA, 23 bp, Artificial Sequence
  IKBKE (IKK) sgRNA target site
tgcatcgcgacatcaagccgggg

SEQ ID NO: 18 - DNA, 23 bp, Artificial Sequence
  CHUK (IKK) sgRNA target site
tagtttagtagtagaacccatgg

SEQ ID NO: 19 - DNA, 23 bp, Artificial Sequence
  IKBKB (IKK) sgRNA target site
gccatggagtactgccaaggagg

SEQ ID NO: 20 - DNA, 23 bp, Artificial Sequence
  IRF7 sgRNA target site
ccgagctgcacgttcctatacgg

SEQ ID NO: 21 - DNA, 23 bp, Artificial Sequence
  IRF3 sgRNA target site
gttactgggtaacatggtgttgg

SEQ ID NO: 22 - DNA, 22 bp, Artificial Sequence
  TRIM33 sgRNA target site
gttatgaacttcacaaattggg

SEQ ID NO: 23 - DNA, 21 bp, Artificial Sequence
  VMP1 sgRNA target site
gaactgccagtttggcccggg

SEQ ID NO: 24 - DNA, 22 bp, Artificial Sequence
  UBA3  sgRNA target site
ggcctaaggagcagccttttgg

SEQ ID NO: 25 - DNA, 23 bp, Artificial Sequence
  NAE1 sgRNA target site
gaattaaatagcgatgtctctgg

SEQ ID NO: 26 - DNA, 21 bp, Artificial Sequence
  NF1 sgRNA target site
gctggtttccttcacgacagg

SEQ ID NO: 27 - DNA, 23 bp, Artificial Sequence
  USP18 sgRNA target site
ggcacagtcaacgcaaatcaagg

SEQ ID NO: 28 - DNA, 23 bp, Artificial Sequence
  NFIC sgRNA target site
gctgctgggcgagaagcccgagg

SEQ ID NO: 29 - DNA, 20 bp, Artificial Sequence
  EFR3A sgRNA target site
gattgctatggaggcactgg

SEQ ID NO: 30 - DNA, 21 bp, Artificial Sequence
  PTPN1 sgRNA target site
gagcagatcgacaagtccggg

SEQ ID NO: 31 - DNA, 23 bp, Artificial Sequence
  MORC3 sgRNA target site
gctgatactgagataccatatgg

SEQ ID NO: 32 - DNA, 23 bp, Artificial Sequence
  TAF5L sgRNA target site
gctgctcaatgacatccttctgg

SEQ ID NO: 33 - DNA, 23 bp, Artificial Sequence
  RNF7 sgRNA target site
ggccatgtggagctgggacgtgg

SEQ ID NO: 34 - DNA, 21 bp, Artificial Sequence
  PI4KA sgRNA target site
gggatagcatacttgcaaagg

SEQ ID NO: 35 - DNA, 23 bp, Artificial Sequence
  IFNAR1 sgRNA target site
gtacattgtataaagaccacagg

SEQ ID NO: 36 - DNA, 23 bp, Artificial Sequence
  IFNAR2 sgRNA target site
tgagtggagaagcacacacgagg

SEQ ID NO: 37 - DNA, 23 bp, Artificial Sequence
  STAT1 sgRNA target site
caggaggtcatgaaaacggatgg

SEQ ID NO: 38 - DNA, 23 bp, Artificial Sequence
  STAT2 sgRNA target site
atcatctcagccaactgggtagg

SEQ ID NO: 39 - DNA, 23 bp, Artificial Sequence
  IFNB1 sgRNA target site
gatgaactttgacatccctgagg

SEQ ID NO: 40 - DNA, 20 bp, Artificial Sequence
  scramble sequence
gctgctccctaacaggacgc

SEQ ID NO: 41 - DNA, 55 bp, Artificial Sequence
  pMCB320fwd 0nt stagger
acactctttccctacacgacgctcttccgatctccttggagaaccaccttgttgg

SEQ ID NO: 42 - DNA, 56 bp, Artificial Sequence
  pMCB320fwd 1nt stagger
acactctttccctacacgacgctcttccgatctcccttggagaaccaccttgttgg

SEQ ID NO: 43 - DNA, 57 bp, Artificial Sequence
  pMCB320fwd 2nt stagger
acactctttccctacacgacgctcttccgatctgcccttggagaaccaccttgttgg

SEQ ID NO: 44 - DNA, 58 bp, Artificial Sequence
  pMCB320fwd 3nt stagger
acactctttccctacacgacgctcttccgatctagcccttggagaaccaccttgttgg

SEQ ID NO: 45 - DNA, 59 bp, Artificial Sequence
  pMCB320fwd 4nt stagger
acactctttccctacacgacgctcttccgatctcaacccttggagaaccaccttgttgg

SEQ ID NO: 46 - DNA, 61 bp, Artificial Sequence
  pMCB320fwd 6nt stagger
acactctttccctacacgacgctcttccgatcttgcaccccttggagaaccaccttgttgg

SEQ ID NO: 47 - DNA, 62 bp, Artificial Sequence
  pMCB320fwd 7nt stagger
acactctttccctacacgacgctcttccgatctacgcaacccttggagaaccaccttgttgg

SEQ ID NO: 48 - DNA, 63 bp, Artificial Sequence
  pMCB320fwd 8nt stagger
acactctttccctacacgacgctcttccgatctgaagacccccttggagaaccaccttgttgg

SEQ ID NO: 49 - DNA, 55 bp, Artificial Sequence
  pMCB320rev2
tgactggagttcagacgtgtgctcttccgatcttcaccacacacggcacttacct

SEQ ID NO: 50 - DNA, 50 bp, Artificial Sequence
  Ad1
aatgatacggcgaccaccgagatctacactcgtcggcagcgtcagatgtg

SEQ ID NO: 51 - DNA, 53 bp, Artificial Sequence
  Ad2.1
caagcagaagacggcatacgagattcgccttagtctcgtgggctcggagatgt

SEQ ID NO: 52 - DNA, 53 bp, Artificial Sequence
  Ad2.2
caagcagaagacggcatacgagatctagtacggtctcgtgggctcggagatgt

SEQ ID NO: 53 - DNA, 53 bp, Artificial Sequence
  Ad2.3
caagcagaagacggcatacgagatttctgcctgtctcgtgggctcggagatgt

SEQ ID NO: 54 - DNA, 53 bp, Artificial Sequence
  Ad2.4
caagcagaagacggcatacgagatgctcaggagtctcgtgggctcggagatgt

SEQ ID NO: 55 - DNA, 53 bp, Artificial Sequence
  Ad2.5
caagcagaagacggcatacgagataggagtccgtctcgtgggctcggagatgt

SEQ ID NO: 56 - DNA, 53 bp, Artificial Sequence
  Ad2.6
caagcagaagacggcatacgagatcatgcctagtctcgtgggctcggagatgt

SEQ ID NO: 57 - DNA, 23 bp, Artificial Sequence
  MORC3 crRNA
gctgatactgagataccatatgg

SEQ ID NO: 58 - DNA, 20 bp, Artificial Sequence
  scramble crRNA
gctgctccctaacaggacgc

SEQ ID NO: 59 - DNA, 62 bp, Artificial Sequence
  WT MRE locus sequence
actgttttatatagtggtcacatcattttataatcctaccaagtgtacattagggttccatt

SEQ ID NO: 60 - DNA, 38 bp, Artificial Sequence
  WT MRE locus sequence
ccatggcacaggctgggaagcagaaaaactagagactc

SEQ ID NO: 61 - DNA, 85 bp, Artificial Sequence
  MRE locus sequence
actgttttatatagtggtcacatcattttataatcctacctacccccccatggcacaggctgggaagcagaaaaactagagactc

SEQ ID NO: 62 - DNA, 79 bp, Artificial Sequence
  MRE locus sequence
actgttttatatagtggtcacatcattttataatcctaccaccatggcacaggctgggaagcagaaaaactagagactc